(19) United States
(12) Patent Application Publication
(10) Pub. No.: US 2026/0098070 A1
(43) Pub. Date: Apr. 9, 2026

(54) SMAGP FUSION MOLECULES AND METHODS OF USE THEREOF

(71) Applicant: DEM BioPharma, Inc., Cambridge, MA (US)

(72) Inventors: Loise M. Francisco-Anderson, Cambridge, MA (US); Sandeep Kumar, Cambridge, MA (US); Sarah Elizabeth Carden, Cambridge, MA (US); Christophe Quéva, Cambridge, MA (US); Nenad Grmusa, Cambridge, MA (US)

(21) Appl. No.: 19/260,916

(22) Filed: Jul. 7, 2025

Related U.S. Application Data
(63) Continuation of application No. PCT/US2024/010530, filed on Jan. 5, 2024.
(60) Provisional application No. 63/478,836, filed on Jan. 6, 2023.
(60) Provisional application No. 63/449,846, filed on Mar. 3, 2023.
(60) Provisional application No. 63/504,348, filed on May 25, 2023.
(60) Provisional application No. 63/510,290, filed on Jun. 26, 2023.

(51) Int. Cl. C07K 14/475; A61K 9/51; A61K 31/7088; A61K 35/76; A61K 38/00; A61K 51/00; A61P 35/00; C12N 15/62; C12N 15/63; G01N 33/68

(52) U.S. Cl. CPC C07K 14/475; A61K 9/51; A61K 31/7088; A61K 35/76; A61K 38/00; A61K 51/00; A61P 35/00; C12N 15/62; C12N 15/63; G01N 33/68; C07K 2317/41; C07K 2317/53; C07K 2319/00; G01N 2333/70596; G01N 2500/02; G01N 2500/20

(57) ABSTRACT

Provided herein are fusion molecules comprising a SMAGP extracellular domain that can modulate leukocyte activity. Also provided are polynucleotides, vectors, and host cells encoding these fusion molecules, and methods of making and using these fusion molecules.

### RELATED APPLICATIONS

This application is continuation of PCT International Application No. PCT/US2024/010530, filed Jan. 5, 2024, which claims benefit of U.S. Provisional Application No. 63/510,290, filed on Jun. 26, 2023, U.S. Provisional Application No. 63/504,348, filed on May 25, 2023, U.S. Provisional Application No. 63/449,846, filed on Mar. 3, 2023, and U.S. Provisional Application No. 63/478,836, filed on Jan. 6, 2023, the contents of which are each herein incorporated by reference in their entireties.

### SEQUENCE LISTING

The content of the electronically submitted Sequence Listing XML (Name: 206873_SL.xml; Size: 73,597 bytes; Created: Jan. 2, 2024) is herein incorporated by reference in its entirety.

### FIELD

This disclosure relates to fusion molecules comprising a SMAGP extracellular domain, methods for making these fusion molecules, and methods of using these fusion molecules to modulate leukocyte activity in a subject.

### BACKGROUND

Phagocytes are immune cells that play a critical role in both the early and late stages of immune responses. Their main role is to circulate and migrate through tissues to ingest and destroy both microbes and cellular debris. However, activation of phagocytes can also contribute to the pathology of autoimmune diseases, such as lupus, rheumatoid arthritis, multiple sclerosis, hemophagocytic lymphohistiocytosis (HLH) and immune thrombocytopenia (ITP).

Accordingly, there is a need for novel compositions and methods that can inhibit phagocyte activation for use in the treatment of autoimmune disease and other inflammatory disorders.

### SUMMARY

This disclosure provides novel fusion molecules comprising a SMAGP extracellular domain that can modulate leukocyte activity. In particular, these fusion molecules can inhibit the phagocytic and inflammatory activities of phagocytes, both of which can contribute to the pathology of autoimmune diseases. Also provided are polynucleotides, vectors, and host cells encoding these fusion molecules, and methods of making and using these fusion molecules.

In one aspect, the instant disclosure provides a fusion molecule comprising a SMAGP extracellular domain (ECD) and a half-life extending moiety.

In some embodiments, the SMAGP ECD is a wild-type human, mouse, or cynomolgus SMAGP ECD.

In some embodiments, the SMAGP ECD comprises an amino acid sequence at least 95% identical to an amino acid sequence selected from the group consisting of SEQ ID NO: 1, 2, and 3.

In some embodiments, the SMAGP ECD comprises an amino acid sequence selected from the group consisting of SEQ ID NO: 1, 2, and 3, or a variant thereof comprising 1-5 amino acid changes.

In some embodiments, the SMAGP ECD comprises an amino acid sequence selected from the group consisting of SEQ ID NO: 1, 2, and 3.

In some embodiments, the half-life extending moiety is an Fc region.

In some embodiments, the Fc region is a human IgG₁, IgG₂, IgG₃, IgG₄, IgA₁, or IgA₂ Fc region.

In some embodiments, the Fc region is a human IgG₁ Fc region.

In some embodiments, the Fc region is a human IgG₁ Fc region comprising D265A and N297A mutations, numbered according to the EU numbering system.

In some embodiments, the Fc region is a human IgG₁ Fc region comprising L234A, L235A, and P329G mutations, numbered according to the EU numbering system.

In some embodiments, the Fc region is a human IgG₁ Fc region comprising S239D and 1332E mutations, numbered according to the EU numbering system.

In some embodiments, the Fc region is a human IgG₄ Fc region.

In some embodiments, the Fc region is a human IgG₄ Fc region comprising an S228P mutation, numbered according to the EU numbering system.

In some embodiments, the Fc region is a mouse IgG₂ₐ Fc region.

In some embodiments, the Fc region is a mouse IgG₂ₐ Fc region comprising L234A, L235A, and P329G mutations, numbered according to the EU numbering system.

In some embodiments, the Fc region comprises an amino acid sequence selected from the group consisting of SEQ ID NOs: 5-18 and 50-57.

In some embodiments, the half-life extending moiety is selected from the group consisting of human serum albumin, a serum albumin-binding moiety, and polyethylene glycol (PEG).

In some embodiments, the serum albumin-binding moiety is an antibody or antigen-binding portion thereof.

In some embodiments, the serum albumin-binding moiety is a VH or VHH.

In some embodiments, the SMAGP ECD is covalently linked to the half-life extending moiety via a linker.

In some embodiments, the linker is a peptide linker.

In some embodiments, the peptide linker is an Fc hinge region or portion thereof.

In some embodiments, the Fc hinge region is a human Fc hinge region or portion thereof.

In some embodiments, the human Fc hinge region is human IgG₁, IgG₂, IgG₃, or IgG₄ hinge region or portion thereof.

In some embodiments, the peptide linker comprises an amino acid sequence selected from the group consisting of SEQ ID NOs: 4, 35-49, and 58-61, or a variant thereof comprising 1-5 amino acid changes.

In some embodiments, the half-life extending moiety is linked to the C-terminus of the SMAGP ECD.

In some embodiments, the half-life extending moiety is linked to the N-terminus of the SMAGP ECD.

In some embodiments, the fusion molecule comprises an amino acid sequence at least 95% identical to an amino acid sequence selected from the group consisting of SEQ ID NOs: 19-34.

In some embodiments, the fusion molecule comprises an amino acid sequence selected from the group consisting of SEQ ID NOs: 19-34.

In some embodiments, the fusion molecule comprises a dimer of two polypeptides, each polypeptide comprising an amino acid sequence selected from the group consisting of SEQ ID NOs: 19-34.

In some embodiments, the fusion molecule further comprises a targeting moiety that binds to a target molecule on a cell.

In some embodiments, the cell is selected from the group consisting of a diseased cell, a senescent cell, a cancer cell, a B cell, a T cell, and a dendritic cell.

In some embodiments, the target molecule is selected from the group consisting of DEP1, NTAL, EBP50, STX4, VAMP3, ARMCX3, B2MG, LANCL1, PLD3, VPS26A, DPP4, SCAMP4, MICA/B, TNFRSF10D/CD264, NOTCH1, NOTCH3, CD36, oxidized Vimentin, ICAM-1, uPAR, DEP1/PTPRJ/CD148, CD264, TNFRSF10D, TRAILR4, and CD26.

In some embodiments, the cell is selected from a diseased cell and a senescent cell.

In some embodiments, the target molecule is selected from the group consisting of ADAM9, B7-H3/CD276, BCMA, CA6, CA9, CD19, CD20, CD22, CD30, CD33, CD37, CD38, CD70, CD79b, CD123, CD138, CD157/BST1, P-cadherin CDH3, CEACAM5, CEACAM6, CLDN6, CLDN18.2, DLL3, EGFR, EGFRvIII, ENPP3, ENTPD2, EpCAM, FGR3, FLT3, FOLR1, GPA33, GPC3, GPNMB, GPRC5D, GUCY2C, Her2, HHLA2, LAMP1, SLC39A6/Liv-1, Mesothelin, MUC16/CA125, MUC17, SLC34A1/NaPi2a, Nectin 4, CD274/PD-L1, PSCA, PSMA/FOLH1, PVR, PVRIg, ROR1, SLITRK6, SSTR2, STEAP1, TROP2, and TMEM97.

In some embodiments, the cell is a cancer cell.

In some embodiments, the target molecule is a B cell surface antigen.

In some embodiments, the target molecule is CD20.

In some embodiments, the targeting moiety is an antibody or antigen-binding fragment thereof.

In some embodiments, the targeting moiety is rituximab or an antigen-binding fragment thereof.

In some embodiments, the fusion molecule further comprises a payload molecule.

In some embodiments, the payload molecule is selected from the group consisting of an mRNA, an miRNA, a circular RNA (cRNA), a tRNA, an siRNA, an sgRNA, an antisense oligonucleotide, a peptide, a virus, a viral RNA genome, a viral DNA genome, a vector, a plasmid, a DNA, and a drug.

In some embodiments, the payload molecule is enclosed in or attached to a nanoparticle.

In some embodiments, the virus is an adeno-associated virus or a lentivirus.

In some embodiments, the vector is a DNA vector.

In another aspect, the instant disclosure provides a fusion molecule comprising a SMAGP extracellular domain (ECD) and a payload molecule.

In some embodiments, the SMAGP ECD is a wild-type human, mouse, or cynomolgus SMAGP ECD.

In some embodiments, the SMAGP ECD comprises an amino acid sequence at least 95% identical to an amino acid sequence selected from the group consisting of SEQ ID NO: 1, 2, and 3.

In some embodiments, the SMAGP ECD comprises an amino acid sequence selected from the group consisting of SEQ ID NO: 1, 2, and 3, or a variant thereof comprising 1-5 amino acid changes.

In some embodiments, the SMAGP ECD comprises an amino acid sequence selected from the group consisting of SEQ ID NO: 1, 2, and 3.

In some embodiments, the payload molecule is selected from the group consisting of an mRNA, an miRNA, a cRNA, a tRNA, an siRNA, an sgRNA, an antisense oligonucleotide, a peptide, a virus, a viral RNA genome, a viral DNA genome, a vector, a plasmid, a DNA, a radionuclide, and a drug. In some embodiments, the virus is an adeno-associated virus or a lentivirus. In some embodiments, the vector is a DNA vector.

In some embodiments, the payload molecule modulates the activity of a cell expressing CLEC10A or CD177.

In some embodiments, the payload molecule is designed or selected to modulate the activity of a neutrophil.

In some embodiments, the payload molecule is selected from the group consisting of a small molecule drug that inhibits the NADPH oxidase complex responsible for the production of ROS, a degranulation inhibitor, and an enhancer of neutrophil apoptosis. In some embodiments, the small molecule drug that inhibits the NADPH oxidase complex responsible for the production of ROS is selected from the group consisting of diphenyleneiodonium and stelazine. In some embodiments, the degranulation inhibitor is nexinhib-20.

In some embodiments, the payload molecule is a nucleic acid molecule encoding a chimeric antigen receptor for a neutrophil.

In some embodiments, the payload molecule is enclosed in or attached to a nanoparticle. In some embodiments, the nanoparticle is a lipid nanoparticle.

In some embodiments, the SMAGP ECD is covalently linked to the payload molecule via a linker.

In some embodiments, the SMAGP ECD is covalently linked to a nanoparticle via a linker, wherein the payload molecule is enclosed in or attached to the nanoparticle.

In some embodiments, the nanoparticle is a lipid nanoparticle and the linker is linked to a lipid component of the lipid nanoparticle.

In some embodiments, the lipid component of the lipid nanoparticle to which the linker is linked is selected from the group consisting of an ionizable lipid, a pegylated lipid, a helper lipid, and cholesterol.

In some embodiments, the helper lipid is selected from the group consisting of 1,2-Distearoyl-sn-glycero-3-phosphorylethanolamine (DSPE); 1,2-distearoyl-sn-glycero-3-phosphocholine (DSPC); 1,2-dioleoyl-sn-glycero-3-phosphoethanolamine (DOPE); and 1,2-dioleoyl-sn-glycero-3-phosphoethanolamine-N-dibenzocyclooctyl (DBCO PE).

In some embodiments, the helper lipid is DBCO PE, wherein the SMAGP ECD comprises an N-terminal azidohomoalanine, and wherein the linker comprises a clickable DARPin that binds to the DBCO PE and the N-terminal azidohomoalanine.

In some embodiments, the pegylated lipid is selected from the group consisting of 1,2-distearoyl-sn-glycero-3-phosphoethanolamine-N-[maleimide(polyethylene glycol)-2000] (DSPE-PEG (2000) Maleimide); 1,2-distearoyl-sn-glycero-3-phosphoethanolamine-N-[maleimide(polyethylene glycol)-5000] (DSPE-PEG (5000) Maleimide); 1,2-dimyristoyl-rac-glycero-3-methoxypolyethylene glycol-2000 (DMG-PEG (2000)); and 2-[(polyethylene glycol)-2000]-N,N-ditetradecylacetamide (ALC-0159).

In some embodiments, the pegylated lipid is DSPE-PEG (2000) Maleimide or DSPE-PEG (5000) Maleimide, and wherein the linker comprises a cysteine fused to the C terminus of the SMAGP ECD that binds to the pegylated lipid.

In some embodiments, the linker is a peptide linker.

In some embodiments, the peptide linker is an Fc hinge region or portion thereof.

In some embodiments, the Fc hinge region is a human Fc hinge region or portion thereof.

In some embodiments, the human Fc hinge region is human IgG₁, IgG₂, IgG₃, or IgG₄ hinge region or portion thereof.

In some embodiments, the peptide linker comprises an amino acid sequence selected from the group consisting of SEQ ID NOs: 4, 35-49, and 58-61, or a variant thereof comprising 1-5 amino acid changes.

In some embodiments, the payload molecule is linked to the C-terminus of the SMAGP ECD.

In some embodiments, the payload molecule is linked to the N-terminus of the SMAGP ECD.

In another aspect, the instant disclosure provides a fusion molecule comprising a SMAGP extracellular domain (ECD) and a targeting moiety that binds to a target molecule on a cell.

In some embodiments, the SMAGP ECD is a wild-type human, mouse, or cynomolgus SMAGP ECD.

In some embodiments, the SMAGP ECD comprises an amino acid sequence at least 95% identical to an amino acid sequence selected from the group consisting of SEQ ID NO: 1, 2, and 3.

In some embodiments, the SMAGP ECD comprises an amino acid sequence selected from the group consisting of SEQ ID NO: 1, 2, and 3, or a variant thereof comprising 1-5 amino acid changes.

In some embodiments, the SMAGP ECD comprises an amino acid sequence selected from the group consisting of SEQ ID NO: 1, 2, and 3.

In some embodiments, the cell is selected from the group consisting of a diseased cell, a senescent cell, a cancer cell, a B cell, a T cell, and a dendritic cell.

In some embodiments, the target molecule is selected from the group consisting of DEP1, NTAL, EBP50, STX4, VAMP3, ARMCX3, B2MG, LANCL1, PLD3, VPS26A, DPP4, SCAMP4, MICA/B, TNFRSF10D/CD264, NOTCH1, NOTCH3, CD36, oxidized Vimentin, ICAM-1, uPAR, DEP1/PTPRJ/CD148, CD264, TNFRSF10D, TRAILR4, and CD26.

In some embodiments, the cell is selected from a diseased cell and a senescent cell.

In some embodiments, the target molecule is selected from the group consisting of ADAM9, B7-H3/CD276, BCMA, CA6, CA9, CD19, CD20, CD22, CD30, CD33, CD37, CD38, CD70, CD79b, CD123, CD138, CD157/BST1, P-cadherin CDH3, CEACAM5, CEACAM6, CLDN6, CLDN18.2, DLL3, EGFR, EGFRvIII, ENPP3, ENTPD2, EpCAM, FGR3, FLT3, FOLR1, GPA33, GPC3, GPNMB, GPRC5D, GUCY2C, Her2, HHLA2, LAMP1, SLC39A6/Liv-1, Mesothelin, MUC16/CA125, MUC17, SLC34A1/NaPi2a, Nectin 4, CD274/PD-L1, PSCA, PSMA/FOLH1, PVR, PVRIg, ROR1, SLITRK6, SSTR2, STEAP1, TROP2, and TMEM97.

In some embodiments, the cell is a cancer cell.

In some embodiments, the target molecule is a B cell surface antigen.

In some embodiments, the target molecule is CD20.

In some embodiments, the targeting moiety is an antibody or antigen-binding fragment thereof.

In some embodiments, the targeting moiety is rituximab or an antigen-binding fragment thereof.

In some embodiments, the targeting moiety is linked to the C-terminus of the SMAGP ECD.

In some embodiments, the targeting moiety is linked to the N-terminus of the SMAGP ECD.

In some embodiments, the fusion molecule binds to a SMAGP ECD receptor on a cell. In some embodiments, the SMAGP ECD is glycosylated.

In some embodiments, the SMAGP ECD is aglycosylated.

In some embodiments, the fusion molecule further comprises a detectable label, optionally wherein the detectable label is selected from the group consisting of an enzyme, a fluorescent label, and a radioisotope.

In another aspect, the instant disclosure provides a fusion molecule comprising an amino acid sequence at least 95% identical to an amino acid sequence selected from the group consisting of SEQ ID NOs: 19-34.

In some embodiments, the fusion molecule comprises an amino acid sequence selected from the group consisting of SEQ ID NOs: 19-34.

In some embodiments, the fusion molecule comprises a dimer of two polypeptides, each polypeptide comprising an amino acid sequence selected from the group consisting of SEQ ID NOs: 19-34.

In another aspect, the instant disclosure provides a polynucleotide comprising a nucleotide sequence encoding any of the fusion molecules provided above and herein.

In another aspect, the instant disclosure provides an expression vector comprising any polynucleotide described above and herein.

In another aspect, the instant disclosure provides a recombinant host cell comprising any polynucleotide or expression vector described above and herein.

In another aspect, the instant disclosure provides a nanoparticle comprising any of the fusion molecules described above and herein, any polynucleotide described above and herein, or any expression vector described above and herein.

In some embodiments, the nanoparticle is a lipid nanoparticle and the polynucleotide is an mRNA.

In another aspect, the instant disclosure provides a method of producing any of the fusion molecules described above and herein comprising culturing any of the host cells described above and herein under conditions such that the fusion molecule is produced.

In another aspect, the instant disclosure provides a pharmaceutical composition comprising any of the fusion proteins described above and herein, or any polynucleotides described above and herein, or any expression vectors described above and herein, and a pharmaceutically acceptable carrier or excipient.

In another aspect, the instant disclosure provides a method of modifying an activity of a leukocyte comprising contacting the leukocyte with any of the fusion proteins described above and herein.

In some embodiments, the leukocyte is selected from the group consisting of myeloid cells, macrophages, Kupffer cells, histiocytes, microglia, osteoclasts, dendritic cells, mast cells, neutrophils, regulatory T cells, tumor-infiltrating regulatory T cells, and granulocytes.

In some embodiments, the leukocyte is a macrophage. In some embodiments, the macrophage is selected from the group consisting of an M0 macrophage, an M1 macrophage, an M2 macrophage, an M2a macrophage, an M2b macrophage, an M2c macrophage, and an M2d macrophage.

In some embodiments, the activity comprises one or more of phagocytosis, cytokine production, chemokine production, antigen presentation, growth factor production, and protease production.

In some embodiments, the activity comprises phagocytosis of a population of cells or cell-like structures. In some embodiments, the population of cells or cell-like structures is selected from the group consisting of cancer cells, immune cells, neurons, red blood cells, and platelets.

In some embodiments, the activity of the leukocyte is increased.

In some embodiments, the activity of the leukocyte is reduced.

In another aspect, the instant disclosure provides a method of treating a disease or disorder in a subject in need thereof, comprising administering to the subject a therapeutically effective amount of any of the fusion molecules described above and herein, any of the polynucleotides described above and herein, any of the expression vectors described above and herein, or any of the pharmaceutical compositions described above and herein.

In some embodiments, the disease or disorder is selected from the group consisting of autoimmune disorder, an autoimmune disorder characterized by excessive phagocytic activity, atherosclerosis, a cancer, an inflammatory disease or disorder, a lymphoproliferative disorder, an infectious disease, macrophage activation syndrome (MAS), a cytokine-related disorder, a central nervous system (CNS) disease, a disease of hyperactivated microglia, a disease of overactivated osteoclasts, osteoporosis, bone degradation and/or metastasis associated with cancer, multiple myeloma bone disease, systemic juvenile idiopathic arthritis, an allergy, a cancer with low PD-L1 expression, a cancer that is refractory to PD-1 and/or PD-L1 inhibitors, a disease or disorder characterized by multinucleated giant cells, and an atopic disease.

In some embodiments, the bone degradation and/or metastasis associated with cancer results from a disease or disorder selected from multiple myeloma, breast cancer, and prostate cancer.

In some embodiments, the autoimmune disorder is lupus, rheumatoid arthritis, multiple sclerosis, hemophagocytic lymphohistiocytosis (HLH), or immune thrombocytopenia (ITP).

In some embodiments, the CNS disease is Alzheimer's disease, schizophrenia, or Huntington's disease.

In some embodiments, the disease or disorder is a lymphoproliferative disorder and the fusion molecule comprises a targeting moiety comprising rituximab or an antigen-binding fragment thereof.

In some embodiments, the disease or disorder is selected from the group consisting of atherosclerosis, a cancer, an inflammatory disease or disorder, and an infectious disease.

In some embodiments, the disease or disorder is selected from the group consisting of H. pylori infection, COVID, severe COVID, IgA nephropathy, and ovarian cancer.

In some embodiments, the disease or disorder characterized by multinucleated giant cells is selected from the group consisting of Langerhans cell histiocytosis and granulomas.

In some embodiments, the disease or disorder is selected from the group consisting of inflammatory bowel disease (IBD), Crohn's disease, ulcerative colitis, acute-on-chronic liver failure (ACLF), COVID, severe COVID, antineutrophil cytoplasmic antibody-associated vasculitis (AAV), autoimmune vasculitis, asthma, acute respiratory distress syndrome (ARDS), chronic obstructive pulmonary disease (COPD), a disease in which regulatory T cells express CD177, Kawasaki disease, septic shock, renal cell carcinoma, hepatocellular carcinoma, breast cancer, lung cancer, and colorectal cancer.

In some embodiments, the method further comprises administering a therapeutically effective amount of a chemotherapeutic agent to the subject. In some embodiments, the chemotherapeutic agent is paclitaxel.

In some embodiments, the method further comprises administering a therapeutically effective amount of an immune checkpoint modulating agent to the subject. In some embodiments, the immune checkpoint modulating agent is an anti-PD-1 agent or an anti-PD-L1 agent.

In some embodiments, the method further comprises administering therapeutically effective amounts of a chemotherapeutic agent and an immune checkpoint modulating agent to the subject. In some embodiments, the chemotherapeutic agent is paclitaxel and the immune checkpoint modulating agent is an anti-PD-1 agent or an anti-PD-L1 agent.

In some embodiments, the disease or disorder is ovarian cancer.

In another aspect, the instant disclosure provides a method of identifying a modulator of the interaction of SMAGP and CLEC10A, the method comprising measuring the binding of SMAGP to CLEC10A in the presence and absence of a test compound, wherein an increase or decrease in the amount of binding of SMAGP to CLEC10A in the presence of the test compound relative to the amount of binding of SMAGP to CLEC10A in the absence of the test compound identifies the test compound as a modulator of the interaction of SMAGP and CLEC10A.

In another aspect, the instant disclosure provides a method of identifying an enhancer of the interaction of SMAGP and CLEC10A, the method comprising measuring the binding of SMAGP to CLEC10A in the presence and absence of a test compound, wherein an increase in the amount of binding of SMAGP to CLEC10A in the presence of the test compound relative to the amount of binding of SMAGP to CLEC10A in the absence of the test compound identifies the test compound as an enhancer of the interaction of SMAGP and CLEC10A.

In another aspect, the instant disclosure provides a method of identifying an inhibitor of the interaction of SMAGP and CLEC10A, the method comprising measuring the binding of SMAGP to CLEC10A in the presence and absence of a test compound, wherein a decrease in the amount of binding of SMAGP to CLEC10A in the presence of the test compound relative to the amount of binding of SMAGP to CLEC10A in the absence of the test compound identifies the test compound as an inhibitor of the interaction of SMAGP and CLEC10A.

In some embodiments, the SMAGP and/or CLEC10A is expressed on the surface of cells.

In some embodiments, the SMAGP and/or CLEC10A is a recombinant protein. In some embodiments, the recombinant protein is a fusion molecule comprising an Fc region.

In another aspect, the instant disclosure provides a method of identifying a modulator of the interaction of SMAGP and CD177, the method comprising measuring the binding of SMAGP to CD177 in the presence and absence of a test compound, wherein an increase or decrease in the amount of binding of SMAGP to CD177 in the presence of the test compound relative to the amount of binding of SMAGP to CD177 in the absence of the test compound identifies the test compound as a modulator of the interaction of SMAGP and CD177.

In another aspect, the instant disclosure provides a method of identifying an enhancer of the interaction of SMAGP and CD177, the method comprising measuring the binding of SMAGP to CD177 in the presence and absence of a test compound, wherein an increase in the amount of binding of SMAGP to CD177 in the presence of the test compound relative to the amount of binding of SMAGP to CD177 in the absence of the test compound identifies the test compound as an enhancer of the interaction of SMAGP and CD177.

In another aspect, the instant disclosure provides a method of identifying an inhibitor of the interaction of SMAGP and CD177, the method comprising measuring the binding of SMAGP to CD177 in the presence and absence of a test compound, wherein a decrease in the amount of binding of SMAGP to CD177 in the presence of the test compound relative to the amount of binding of SMAGP to CD177 in the absence of the test compound identifies the test compound as an inhibitor of the interaction of SMAGP and CD177.

In some embodiments, the SMAGP and/or CD177 is expressed on the surface of cells.

In some embodiments, the SMAGP and/or CD177 is a recombinant protein. In some embodiments, the recombinant protein is a fusion molecule comprising an Fc region.

In some embodiments, the test compound is selected from the group consisting of a small molecule, a polypeptide, or a nucleic acid. In some embodiments, the polypeptide is an antibody. In some embodiments, the nucleic acid is a DNA or RNA aptamer.

In some embodiments, the amount of binding is measured using enzyme linked immunosorbent assay (ELISA), surface plasmon resonance (SPR), or flow cytometry.

In another aspect, the instant disclosure provides a method of treating a subject that has a tumor with an elevated level of CLEC10A expression as compared to a reference level of CLEC10A, the method comprising administering to the subject a therapeutically effective amount of a compound that antagonizes the binding of CLEC10A to SMAGP. In some embodiments, the compound is any of the fusion molecules described above and herein, any of the polynucleotides described above and herein, any of the expression vectors described above and herein, or any of the pharmaceutical compositions described above and herein.

In another aspect, the instant disclosure provides a method of identifying a subject who will benefit from treatment with a fusion molecule that antagonizes the binding of CLEC10A to SMAGP, the method comprising determining the level of CLEC10A expression in a biological sample from the subject relative to a reference level of CLEC10A, wherein a higher level of CLEC10A expression in the biological sample compared to the reference level indicates that the subject will benefit from the treatment. In some embodiments, the biological sample is a tumor sample. In some embodiments, the level of CLEC10A expression in the biological sample is determined by immunohistochemistry, fluorescence in situ hybridization (FISH), or chromogenic in-situ hybridization (CISH). In some embodiments, the CLEC10A is expressed on the surface of a myeloid cell and the SMAGP is expressed on the surface of a tumor cell.

In another aspect, the instant disclosure provides a method of identifying a subject who will benefit from treatment with a fusion molecule comprising a SMAGP extracellular domain (ECD), comprising measuring a level of CD177 expression in a biological sample from the subject and comparing to a reference level of CD177, wherein a higher level of CD177 expression compared to the reference level indicates the subject will benefit from the treatment. In some embodiments, the biological sample is a tumor sample. In some embodiments, the level of CD177 expression in the biological sample is determined by immunohistochemistry, fluorescence in situ hybridization (FISH), or chromogenic in-situ hybridization (CISH). In some embodiments, the CD177 is expressed on the surface of a neutrophil or a regulatory T cell and the SMAGP is expressed on the surface of a tumor cell.

In some embodiments, the method further comprises administering to the subject a therapeutically effective amount of any of the fusion molecules described above and herein, any of the polynucleotides described above and herein, any of the expression vectors described above and herein, or any of the pharmaceutical compositions described above and herein.

In another aspect, the instant disclosure provides a fusion molecule described above and herein, a polynucleotide described above and herein, an expression vector described above and herein, a nanoparticle described above and herein, or a composition described above and herein for use in modifying an activity of a leukocyte.

In another aspect, the instant disclosure provides a fusion molecule described above and herein, a polynucleotide described above and herein, an expression vector described above and herein, a nanoparticle described above and herein, or a composition described above and herein for use in medicine.

In another aspect, the instant disclosure provides a fusion molecule described above and herein, a polynucleotide described above and herein, an expression vector described above and herein, a nanoparticle described above and herein, or a composition described above and herein for use in treating a disease or disorder in a subject in need thereof.

In another aspect, the instant disclosure provides a use of a fusion molecule described above and herein, a polynucleotide described above and herein, an expression vector described above and herein, a nanoparticle described above and herein, or a composition described above and herein in the manufacture of a medicament for the treatment of a disease or disorder in a subject in need thereof.

### BRIEF DESCRIPTION OF THE DRAWINGS

FIG. 1A is a schematic of the structure of a SMAGP extracellular domain (ECD)-IgG1 Fc fusion molecule. SMAGP ECD is depicted in light gray; Fc is depicted in dark gray. FIG. 1B is an HPLC-SEC chromatogram of purified human SMAGP ECD-hIgG1 Fc (hSMAGP ECD-hIgG1 Fc) and accompanying data table.

FIG. 2A and FIG. 2B are flow cytometry histograms showing the binding of hIgG1 Fc control, hSMAGP ECD-hIgG1 Fc, and cynomolgus SMAGP ECD-hIgG1 Fc (cSMAGP ECD-hIgG1 Fc) to human macrophages from two different donors. FIG. 2C and FIG. 2D are flow cytometry histograms showing the binding of hIgG1 Fc control and hSMAGP ECD-hIgG1 Fc to human neutrophils (FIG. 2C) and human monocytes (FIG. 2D).

FIG. 3A and FIG. 3B are bar graphs showing the extent of phagocytosis of Ramos tumor cells by macrophages from two different donors in the presence of anti-CD47 antibody and hSMAGP ECD-hIgG1 Fc or hIgG1 Fc control at the indicated concentrations. Additional controls include macrophages cocultured with Ramos SMAGP-overexpressing cells in the presence of anti-CD47 antibody and macrophages cocultured with Ramos parental cell lines in the absence of anti-CD47 antibody.

FIG. 4A and FIG. 4B are bar graphs showing the extent of Ramos tumor cell recovery following the inhibition of phagocytosis assays depicted in FIG. 3A and FIG. 3B.

FIG. 5A is a bar graph showing the extent of phagocytosis of A375 tumor cells by macrophages from three different donors in the presence of anti-CD47 antibody and hSMAGP ECD-hIgG1 Fc or hIgG1 Fc control at the indicated concentrations, quantified at 4 hours. FIG. 5B is a graph showing a time course of the extent of phagocytosis of A375 tumor cells by macrophages from a representative donor in the presence of anti-CD47 antibody and hSMAGP ECD-hIgG1 Fc or hIgG1 Fc control at the indicated concentrations.

FIG. 6A is a bar graph showing the extent of phagocytosis of Ramos tumor cells by macrophages from three different donors in the presence of rituximab and hIgG1 Fc control, hSMAGP ECD-hIgG1 Fc, CD24 ECD-hIgG1 Fc, or CD47 ECD-hIgG1 Fc at the indicated concentrations, quantified at 3 hours. FIG. 6B shows the extent of Ramos tumor cell recovery following inhibition of phagocytosis.

FIG. 7 is a bar graph showing the extent of phagocytosis of Ramos tumor cells by macrophages from three different donors in the presence of rituximab and hIgG1-Fc, hSMAGP ECD-hIgG1 Fc, hIgG1 LALA PG, hSMAGP ECD-hIgG1 LALA PG Fc, hIgG4, and hSMAGP ECD-hIgG4 at the indicated concentrations.

FIG. 8A is a bar graph showing the extent of phagocytosis of Ramos tumor cells by macrophages in the presence of MCSF plus polarization stimulation as indicated (M0, without polarization stimulation; M1, induced by LPS and IFN-γ; M2, induced by IL-10; M2, induced by TGF-β; M2, induced by TGF-β and IL-10; and M2, induced by TGF-β, IL-10, and IL-4) treated with hSMAGP ECD-hIgG1 Fc or hIgG1 Fc control as indicated and quantified at 2 hrs. FIG. 8B is a graph showing a time course of the extent of phagocytosis of Ramos cells by M1 polarized macrophages (induced by LPS and IFN-γ) treated with hSMAGP ECD-hIgG1 Fc or hIgG1 Fc control as indicated and quantified over 21 hrs.

FIG. 9A is a graph showing a time course of the extent of phagocytosis of red blood cells (RBCs) by macrophages from a representative donor in the presence of anti-RBC antibody and hIgG1 Fc control, hSMAGP ECD-hIgG1 Fc, or media alone, at the indicated concentrations. FIG. 9B is a bar graph showing the extent of phagocytosis of RBCs by macrophages from three donors in the presence of anti-RBC antibody and hIgG1 Fc control, hSMAGP ECD-hIgG1 Fc, or media alone at 10 hours. FIG. 9C is a set of three graphs showing dose-response curves corresponding to the area under the curve of total red object area for the time course plotted versus indicated opsonizing antibody concentration for each of the three donors.

FIG. 10 is a set of three graphs showing the percent of live RBCs remaining after phagocytosis by macrophages from three donors in the presence of either hIgG1 Fc control or hSMAGP ECD-hIgG1 Fc at the indicated concentrations.

FIG. 11A, FIG. 11B, and FIG. 11C are bar graphs showing the extent of secretion of TNFα (FIG. 11A), IL-12p70 (FIG. 11B), and IL-6 (FIG. 11C) after treatment with media alone, hIgG1 Fc control, or hSMAGP ECD-hIgG1 Fc, plus or minus LPS overnight.

FIG. 12A-FIG. 12C are bar graphs showing the extent of binding of each of a panel of lectins to hSMAGP ECD-hIgG1 Fc (FIG. 12A), cSMAGP ECD-hIgG1 Fc (FIG. 12B), and buffer (FIG. 12C) plus streptavidin Cy3 in the presence of cations. The identities of the tested lectins are not shown, with the exception of CLEC10A/MGL. NC=immobilization buffer; PC1=positive control (consisting of Amine-PEG-biotin); PC2=Human IgG; PC3=Mouse IgG; M=marker.

FIG. 13A-FIG. 13C are bar graphs showing the extent of binding of each of a panel of lectins to hSMAGP ECD-hIgG1 Fc (FIG. 13A), cSMAGP ECD-hIgG1 Fc (FIG. 13B), and buffer (FIG. 13C) plus streptavidin Cy3 in the absence of cations. The identities of the tested lectins are not shown, with the exception of CLEC10A/MGL. NC=immobilization buffer; PC1=positive control (consisting of Amine-PEG-biotin); PC2=Human IgG; PC3=Mouse IgG; M=marker.

FIG. 14 is a flow cytometry histogram showing the binding of hSMAGP ECD-hIgG1 Fc to CLEC10A-overexpressing cells or control cells.

FIG. 15 is a bar graph showing the extent of binding of hSMAGP ECD-hIgG1 Fc to recombinant CLEC10A ECD or negative control in an ELISA assay.

FIG. 16A and FIG. 16B are images of slides of membrane proteins expressed in HEK293 cells and probed with hSMAGP ECD-hIgG1 Fc (FIG. 16A) or CTLA4-hFc (control) (FIG. 16B).

FIG. 17 is a flow cytometry histogram showing the binding of hSMAGP ECD-hIgG1 Fc to CD177-overexpressing cells or control cells.

FIG. 18 is a bar graph showing the extent of binding of hSMAGP ECD-hIgG1 Fc to recombinant CD177 ECD or negative control in an ELISA assay.

FIG. 19 is a bar graph showing the extent of binding of aglycosylated scrambled hSMAGP ECD peptide, aglycosylated hSMAGP ECD peptide, and aglycosylated hSMAGP ECD peptide, in the presence of anti-CD177 antibody, to recombinant CD177 ECD or negative control in an ELISA assay.

FIG. 20A and FIG. 20B are sets of flow cytometry histograms showing representative expression of CD177 (FIG. 20A) and CLEC10A (FIG. 20B) on M0 and M2a/c human macrophages.

FIG. 21A and FIG. 21B are sets of flow cytometry histograms showing representative expression of CD177 (FIG. 21A) and CLEC10A (FIG. 21B) on neutrophils, monocytes, B cells, T cells, NK cells, and NKT cells.

### DETAILED DESCRIPTION

This disclosure provides novel fusion molecules comprising a SMAGP extracellular domain that can modulate leukocyte activity. In particular, these fusion molecules can inhibit the phagocytic and inflammatory activities of phagocytes, both of which can contribute to the pathology of autoimmune diseases. Also provided are polynucleotides, vectors, and host cells encoding these fusion molecules, and methods of making and using these fusion molecules.

### Definitions

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an,” and “the” include plural referents unless the context clearly dictates otherwise. In this application, the use of “or” means “and/or” unless stated otherwise. Furthermore, use of the term “including” as well as other forms, such as “include,” “includes,” and “included,” is not limiting. The section headings used herein are for organizational purposes only and are not to be construed as limiting the subject matter described.

As used herein, the term “SMAGP” refers to Small Cell Adhesion Glycoprotein. An exemplary human SMAGP is set forth in NCBI Reference Sequence: NM_001033873.1. An exemplary human SMAGP extracellular domain (ECD) is set forth in SEQ ID NO: 1.

As used herein, the term “half-life extending moiety” refers to any molecule that when linked to a SMAGP ECD can extend the serum half-life of the SMAGP ECD.

As used herein, the term “Fc region” refers to the portion of an immunoglobulin formed by the Fc domains of its two heavy chains. In some embodiments, the Fc region is formed from an IgG1 heavy chain constant region. In some embodiments, residues 356 and 358 are D and L, respectively, numbered according to the EU numbering system. In some embodiments, residues 356 and 358 are E and M, respectively, numbered according to the EU numbering system. In some embodiments, the IgG1 heavy chain constant region comprises a G1m1(a), Glm2(x), Glm3(f), or Glm17(z) allotype. Sec, e.g., Jefferis and Lefranc (2009) mAbs 1 (4): 332-338, and de Taeye et al. (2020) Front Immunol. 11:740, incorporated herein by reference in their entirety.

As used herein, the term “variant Fc region” refers to a variant of an Fc region with one or more alteration(s) relative to a native Fc region. Alterations can include amino acid substitutions, additions and/or deletions, linkage of additional moieties, and/or alteration of the native glycans. The term encompasses heterodimeric Fc regions where each of the constituent Fc domains is different. The term also encompasses single chain Fc regions where the constituent Fc domains are linked together by a linker moiety.

As used herein, the term “Fc domain” refers to the portion of a single antibody heavy chain comprising both the CH2 and CH3 domains of the antibody. In some embodiments, the Fc domain comprises at least a portion of a hinge region (e.g., the amino acid sequence EPKSX (SEQ ID NO: 4), wherein X is cysteine or serine), a CH2 domain, and a CH3 domain.

As used herein, the term “hinge region” refers to the portion of an antibody heavy chain molecule that joins the CH1 domain to the CH2 domain. In some embodiments, the hinge region is from an IgG1 antibody. In some embodiments, the hinge region includes the upper hinge region, the core hinge region, and/or the lower hinge region, e.g., as described in Chiu et al. (2019) Antibodies (Basel) 8 (4): 55 (which is hereby incorporated by reference in its entirety). In some embodiments, the hinge region comprises the amino acid sequence EPKSX (SEQ ID NO: 4), wherein X is cysteine or serine. In some embodiments, the hinge region comprises the amino acid sequence EPKSXDKTHT (SEQ ID NO: 58), wherein X is cysteine or serine. In some embodiments, the hinge region comprises the amino acid sequence EPKSXDKTHTCPPCP (SEQ ID NO: 59), wherein X is cysteine or serine. In some embodiments, the hinge region comprises the amino acid sequence EPKSXDKTHTCPPCPAPELL (SEQ ID NO: 60), wherein X is cysteine or serine. In some embodiments, the hinge region comprises the amino acid sequence EPKSXDKTHTCPPCPAPELLGGP (SEQ ID NO: 61), wherein X is cysteine or serine.

As used herein, the term “CH2 domain” refers to the portion of an antibody heavy chain that generally corresponds to residues 231-340 of human IgG₁, numbered according to the EU numbering system. In some instances, a CH2 domain may begin after residue 231 of human IgG₁, e.g., spanning residues 236-340, 237-340, or 239-340, numbered according to the EU numbering system.

As used herein, the term “CH3 domain” refers to the portion of an antibody heavy chain that corresponds to residues 341-446 or 341-447 of human IgG₁, numbered according to the EU numbering system.

As used herein, the term “serum albumin-binding moiety” refers to any molecule specifically binds to serum albumin (e.g., human serum albumin). Examples of serum albumin-binding moieties include polypeptides derived from antibodies, such as Fab fragments, F(ab′)₂ fragments, disulfide-linked Fvs (sdFv), single-chain Fvs (scFv), CDRs, VH domains, VL domains, single-domain antibodies (sdAb), VHH domains, camelid antibodies, and antigen-binding fragments of any of the above. The term also encompasses synthetic antigen-binding proteins or antibody mimetic proteins such as, for example, affibodies, lipocalins, and DARPins.

As used herein, the terms “antibody” and “antibodies” include full-length antibodies, antigen-binding fragments of full-length antibodies, and molecules comprising antibody CDRs, VH domains, or VL domains. Examples of antibodies include monoclonal antibodies, recombinantly produced antibodies, monospecific antibodies, multi-specific antibodies (including bispecific antibodies), human antibodies, humanized antibodies, chimeric antibodies, immunoglobulins, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain-antibody heavy chain pair, intrabodies, heteroconjugate antibodies, antibody-drug conjugates, single-domain antibodies (sdAb), monovalent antibodies, single chain antibodies or single-chain Fvs (scFv), camelid antibodies, single-domain antibodies (sdAb), humanized antibodies, VHH domains, Fab fragments, F(ab′)₂ fragments, disulfide-linked Fvs (sdFv), anti-idiotypic (anti-Id) antibodies (including, e.g., anti-anti-Id antibodies), and antigen-binding fragments of any of the above. Antibodies can be of any isotype (e.g., IgG, IgE, IgM, IgD, IgA, or IgY), any subclass (e.g., IgG₁, IgG₂, IgG₃, IgG₄, IgA₁, or IgA₂), or species (e.g., mouse IgG₂ₐ or IgG_{2b}) of immunoglobulin molecule.

As used herein, the terms “VH” and “VH domain” are used interchangeably to refer to a heavy chain variable region of an antibody.

As used herein, the terms “VL” and “VL domain” are used interchangeably to refer to a light chain variable region of an antibody.

As used herein, the terms “VHH” and “VHH domain” are used interchangeably to refer to a variable region of a heavy chain antibody (e.g., a camelid heavy chain antibody) or a humanized variant thereof.

The determination of “percent identity” between two sequences (e.g., amino acid sequences or nucleic acid sequences) can be accomplished using a mathematical algorithm. A specific, non-limiting example of a mathematical algorithm utilized for the comparison of two sequences is the algorithm of Karlin S & Altschul S F, (1990) PNAS 87:2264-2268, modified as in Karlin S & Altschul S F, (1993) PNAS 90:5873-5877, each of which is herein incorporated by reference in its entirety. Such an algorithm is incorporated into the NBLAST and XBLAST programs of Altschul S F et al., (1990) J Mol Biol 215:403, which is herein incorporated by reference in its entirety. BLAST nucleotide searches can be performed with the NBLAST nucleotide program parameters set, e.g., at score=100, wordlength=12 to obtain nucleotide sequences homologous to a nucleic acid molecule described herein. BLAST protein searches can be performed with the XBLAST program parameters set, e.g., at score=50, wordlength=3 to obtain amino acid sequences homologous to a protein molecule described herein. To obtain gapped alignments for comparison purposes, Gapped BLAST can be utilized as described in Altschul S F et al., (1997) Nuc Acids Res 25:3389-3402, which is herein incorporated by reference in its entirety. Alternatively, PSI BLAST can be used to perform an iterated search which detects distant relationships between molecules. Id. When utilizing BLAST, Gapped BLAST, and PSI BLAST programs, the default parameters of the respective programs (e.g., of XBLAST and NBLAST) can be used (see, e.g., National Center for Biotechnology Information (NCBI) on the worldwide web, ncbi.nlm.nih.gov). Another specific, non-limiting example of a mathematical algorithm utilized for the comparison of sequences is the algorithm of Myers and Miller, (1988) CABIOS 4:11-17, which is herein incorporated by reference in its entirety. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. The percent identity between two sequences can be determined using techniques similar to those described above, with or without allowing gaps. In calculating percent identity, typically only exact matches are counted.

As used herein, the term “payload molecule” refers to any molecule that can be linked a fusion molecule (e.g., for delivery to a cell by binding of the fusion molecule to the cell).

As used herein, the term “polynucleotide” as used herein refers to a polymer of DNA or RNA. The polynucleotide sequence can be single-stranded or double-stranded; contain natural, non-natural, or altered nucleotides; and contain a natural, non-natural, or altered internucleotide linkage, such as a phosphoroamidate linkage or a phosphorothioate linkage, instead of the phosphodiester found between the nucleotides of an unmodified polynucleotide sequence. Polynucleotide sequences include, but are not limited to, all polynucleotide sequences which are obtained by any means available in the art, including, without limitation, recombinant means, e.g., the cloning of polynucleotide sequences from a recombinant library or a cell genome, using ordinary cloning technology and polymerase chain reaction, and the like, and by synthetic means.

As used herein, the term “linked” refers to a physical linkage (e.g., directly or indirectly linked) between amino acid sequences (e.g., different segments, regions, or domains). Linked regions, domains, and segments of the fusion molecules of the disclosure may be contiguous or non-contiguous (e.g., linked to one another through a linker). In some embodiments, linkages are covalent. In some embodiments, linkages are non-covalent.

As used herein, the term “EU numbering system” refers to the EU numbering convention for the constant regions of an antibody, as described in Edelman, G. M. et al., Proc. Natl. Acad. USA, 63, 78-85 (1969) and Kabat et al., Sequences of Proteins of Immunological Interest, U.S. Dept. Health and Human Services, 5th edition, 1991, each of which is herein incorporated by reference in its entirety.

As used herein, the terms “treat,” “treating,” and “treatment” refer to therapeutic or preventative measures described herein. In some embodiments, the methods of “treatment” employ administration of a fusion protein to a subject having a disease or disorder, or predisposed to having such a disease or disorder, in order to prevent, cure, delay, reduce the severity of, or ameliorate one or more symptoms of the disease or disorder or recurring disease or disorder, or in order to prolong the survival of a subject beyond that expected in the absence of such treatment.

As used herein, the term “effective amount” in the context of the administration of a therapy to a subject refers to the amount of a therapy that achieves a desired prophylactic or therapeutic effect.

As used herein, the term “subject” includes any human or non-human animal. In one embodiment, the subject is a human or non-human mammal. In one embodiment, the subject is a human.

### Fusion Molecules

The disclosure provides a fusion molecule comprising a SMAGP extracellular domain (ECD) and one or more moieties selected from the group consisting of a half-life extending moiety, a targeting moiety, and a payload molecule. In some embodiments, the disclosure provides a fusion molecule comprising a SMAGP ECD and a half-life extending moiety. In some embodiments, the disclosure provides a fusion molecule comprising a SMAGP ECD and a targeting moiety. In some embodiments, the disclosure provides a fusion molecule comprising a SMAGP ECD and a payload molecule.

### SMAGP Extracellular Domains

The disclosure provides fusion molecules comprising a SMAGP extracellular domain (ECD). SMAGP ECDs disclosed herein may be derived from any mammalian species, including for example, a human, a rodent (e.g., a mouse, rat, rabbit, guinea pig), or a non-human primate (e.g., chimpanzee, macaque) species. In some embodiments, the SMAGP ECD is a wild-type human, mouse, or cynomolgus SMAGP ECD. In some embodiments, the SMAGP ECD is a variant of a wild-type human, mouse, or cynomolgus SMAGP ECD. Exemplary SMAGP ECDs are set forth in Table 1, below.

TABLE 1
Exemplary SMAGP ECD Amino Acid Sequences.

| Description | Amino Acid Sequence | SEQ ID NO: |
|---|---|---|
| Human SMAGP | MTSLLTTPSPREELMTTP | 1 |
| ECD | ILQPTEALSPEDGASTAL | |
| Mouse SMAGP | MNNLPATPSPEELMTTPV | 2 |
| ECD | FQAPETLSPQAEEASTAL | |
| Cynomolgus | MTSLLTTPSPRELMTTPI | 3 |
| SMAGP ECD | LRPTEALSPEDGASTAL | |

In some embodiments, the disclosure provides a fusion molecule comprising a SMAGP ECD comprising an amino acid sequence at least 70%, 75%, 80%, 85%, 90%, 95%, 96%, 97%, 98%, or 99% identical to an amino acid sequence selected from the group consisting of SEQ ID NOs: 1, 2, and 3. In some embodiments, the SMAGP ECD comprises an amino acid sequence that is at least 95% identical to an amino acid sequence selected from the group consisting of SEQ ID NOs: 1, 2, and 3.

In some embodiments, the disclosure provides a fusion molecule comprising a SMAGP ECD comprising a variant of an amino acid sequence selected from the group consisting of SEQ ID NOs: 1, 2, and 3 comprising 1, 2, 3, 4, or 5 amino acid changes (e.g., substitutions, insertions, and/or deletions). In some embodiments, the SMAGP ECD comprises an amino acid sequence selected from the group consisting of SEQ ID NOs: 1, 2, and 3. In some embodiments, the amino acid sequence of the SMAGP ECD consists of an amino acid sequence selected from the group consisting of SEQ ID NOs: 1, 2, and 3.

In some embodiments, the disclosure provides a fusion molecule comprising a SMAGP ECD comprising a variant of an amino acid sequence selected from the group consisting of SEQ ID NOs: 1, 2, and 3, wherein one or more amino acids from the N-terminus are absent. In some embodiments, the SMAGP ECD comprises a variant of an amino acid sequence selected from the group consisting of SEQ ID NOs: 1, 2, and 3, wherein the N-terminal methionine is absent. In some embodiments, the SMAGP ECD comprises a variant of an amino acid sequence selected from the group consisting of SEQ ID NOs: 1, 2, and 3, wherein one or more amino acids from the C-terminus are absent.

### Half-Life Extending Moieties

In some embodiments, the disclosure provides a fusion molecule comprising a SMAGP ECD and a half-life extending moiety. The half-life extending moiety can linked to the N-terminus or N-terminus of the SMAGP ECD.

In some embodiments, the half-life extending moiety comprises an Fc region or a derivative thereof. Any Fc region can be used in the fusion molecules disclosed herein. In certain embodiments, the Fc region is a human immunoglobulin (Ig) of any isotype (e.g., IgG, IgE, IgM, IgD, IgA, and IgY) of immunoglobulin molecule, any class (e.g., IgG₁, IgG₂, IgG₃, IgG₄, IgA₁, and IgA₂), or any subclass (e.g., IgG₂a and IgG₂b) of immunoglobulin molecule. In an embodiment, the Fc region is an IgG Fc region (e.g., a human IgG region). In an embodiment, the Fc region is an IgG₁ Fc region (e.g., a human IgG₁ region). In an embodiment, the Fc region is a chimeric Fc region comprising portions of several different Fc regions. Suitable examples of chimeric Fc regions are set forth in US 2011/0243966A1, which is incorporated herein by reference in its entirety. A variety of Fc region gene sequences (e.g., human constant region gene sequences) are available in the form of publicly accessible deposits.

In some embodiments, the disclosure provides a fusion molecule comprising a SMAGP ECD and an Fc region, wherein the Fc region is a human IgG₁, IgG₂, IgG₃, IgG₄, IgA₁, or IgA₂ Fc region. The Fc region can be of any allotype or isoallotype. In some embodiments, the disclosure provides a fusion molecule comprising a SMAGP ECD and an Fc region, wherein the Fc region is an IgG₁ Fc region of an allotype selected from the group consisting of G1m1(a), Glm2(x), Glm3(f), and Glm17(z). Sec, e.g., Jefferis and Lefranc (2009) mAbs 1 (4): 332-338. In some embodiments, the disclosure provides a fusion molecule comprising a SMAGP ECD and an Fc region, wherein the Fc region is an IgG₂ Fc region of any allotype or isoallotype. In some embodiments, the Fc region is an IgG₂ Fc region of a G2m23 (n) allotype. (Id.). In some embodiments, the disclosure provides a fusion molecule comprising a SMAGP ECD and an Fc region, wherein the Fc region is an IgG₃ Fc region of any allotype or isoallotype. In some embodiments, the IgG₃ Fc region of an allotype selected from the group consisting of G3m21 (g1), G3m28 (g5), G3m11 (b0), G3m5 (b1), G3m13 (b3), G3m14 (b4), G3m10 (b5), G3m15 (s), G3m16 (t), G3m6 (c3), G3m24 (c5), G3m26 (u), and G3m27 (v). (Id.). In some embodiments, the disclosure provides a fusion molecule comprising a SMAGP ECD and an Fc region, wherein the Fc region is an IgA Fc region of any allotype or isoallotype. In some embodiments, the IgA Fc region of an allotype selected from the group consisting of A2 m1 and A2m2. (Id.).

In some embodiments, the Fc region is a variant of a wild-type Fc region. In some embodiments, the variant Fc region varies from the Fc region of any native immunoglobulin. In some embodiments, the native immunoglobulin is a human immunoglobulin. In some embodiments, the immunoglobulin is IgA, IgD, IgE, or IgG. In some embodiments, the immunoglobulin is IgG. In some embodiments, the immunoglobulin is human IgA, human IgD, human IgE, or human IgG. In some embodiments, the immunoglobulin is human IgG. In some embodiments, the IgG is IgG1, IgG2, IgG3, or IgG4. In some embodiments, the human IgG is human IgG1, human IgG2, human IgG3, or human IgG4. In some embodiments, the variant Fc region varies from the human IgG1 Fc region. In some embodiments, the human IgG1 Fc region comprises a G1m1(a), Glm2(x), Glm3(f), or Glm17 (z) allotype. In some embodiments, the native immunoglobulin is a mouse immunoglobulin. In some embodiments, the mouse IgG is mouse IgG1, mouse IgG2a, mouse IgG2b, or mouse IgG3. In some embodiments, the variant Fc region varies from the mouse IgG2a Fc region.

In some embodiments, the disclosure provides a fusion molecule comprising a SMAGP ECD and an Fc region, wherein the Fc region includes CH2 and CH3 domains and at least a portion of a hinge region. In specific embodiments, the SMAGP ECD is directly linked to the hinge region. In more specific embodiments, the hinge region comprises the amino acid sequence EPKSX (SEQ ID NO: 4), wherein X is cysteine or serine.

The Fc region can be linked to the N-terminus of the SMAGP ECD or the C-terminus of the SMAGP ECD. The Fc region can be linked directly to the SMAGP ECD or it can be linked to the SMAGP ECD through a linker. In some embodiments, the linker is a peptide linker. In some embodiments, the peptide linker comprises or consists of 1, 2, 3, 4, 5, 6, 7, 8, 9, or 10 amino acids. In some embodiments, the peptide linker comprises or consists of 1, 2, 3, 4, or 5 amino acids. In some embodiments, the peptide comprises or consists of 4 or 5 amino acids. Any peptide linker can be used. Exemplary peptide linkers are described herein.

In some embodiments, one or more mutations (e.g., amino acid substitutions) are introduced into the Fc region of a fusion molecule described herein to alter one or more functional properties of the fusion molecule, such as serum half-life, complement fixation, Fc receptor binding, and/or antigen-dependent cellular cytotoxicity. Mutations can be introduced into the CH2 domain, CH3 domain, and/or the hinge region.

In some embodiments, one or more mutations (e.g., amino acid substitutions) are introduced into the hinge region such that the number of cysteine residues in the hinge region are altered (e.g., increased or decreased) as described in, e.g., U.S. Pat. No. 5,677,425, herein incorporated by reference in its entirety. The number of cysteine residues in the hinge region may be altered to, e.g., alter (e.g., increase or decrease) the stability of the fusion molecule.

In some embodiments, one or more amino acid mutations (e.g., substitutions, insertions, or deletions) are introduced into an Fc region to alter (e.g., decrease or increase) half-life of the fusion molecule in vivo. See, e.g., International Publication Nos. WO 02/060919; WO 98/23289; and WO 97/34631; and U.S. Pat. Nos. 5,869,046, 6,121,022, 6,277,375, and 6,165,745, all of which are herein incorporated by reference in their entireties, for examples of mutations that will alter (e.g., decrease or increase) the half-life of an antibody in vivo. In some embodiments, one or more amino acid mutations (e.g., substitutions, insertions, or deletions) are introduced into an Fc region to increase the half-life of the fusion molecule in vivo.

In some embodiments, one or more mutations (e.g., amino acid substitutions) are introduced into the Fc region to increase or decrease the affinity of the fusion molecule for an Fc receptor (e.g., an activated Fc receptor) on the surface of an effector cell. Mutations in the Fc region of a fusion molecule that increase or decrease the affinity of a fusion molecule for an Fc receptor and techniques for introducing such mutations into the Fc receptor or fragment thereof are known to one of skill in the art. Examples of mutations in the Fc receptor of an antibody that can be made to alter the affinity of the antibody for an Fc receptor are described in, e.g., Smith P et al., (2012) PNAS 109:6181-6186, U.S. Pat. No. 6,737,056, and International Publication Nos. WO 02/060919; WO 98/23289; and WO 97/34631, all of which are herein incorporated by reference in their entireties.

In a further embodiment, one or more amino acid substitutions are introduced into an IgG constant domain Fc region to alter the effector function(s) of the fusion molecule. For example, one or more amino acids selected from amino acid residues 234, 235, 236, 237, 239, 243, 267, 292, 297, 300, 318, 320, 322, 328, 330, 332, and 396, numbered according to the EU numbering system, can be replaced with a different amino acid residue such that the fusion molecule has an altered affinity for an effector ligand but retains the function of the parent molecule. The effector ligand to which affinity is altered can be, for example, an Fc receptor or the C1 component of complement. This approach is described in further detail in U.S. Pat. Nos. 5,624,821 and 5,648,260, each of which is herein incorporated by reference in its entirety. In certain embodiments, the deletion or inactivation (through point mutations or other means) of an Fc region may reduce Fc receptor binding of the circulating fusion molecule thereby increasing tumor localization. See, e.g., U.S. Pat. Nos. 5,585,097 and 8,591,886, each of which is herein incorporated by reference in its entirety, for a description of mutations that delete or inactivate the constant domain and thereby increase tumor localization. In certain embodiments, one or more amino acid substitutions may be introduced into the Fc region of a fusion molecule described herein to remove potential glycosylation sites on the Fc region, which may reduce Fc receptor binding (see, e.g., Shields R L et al., (2001) J Biol Chem 276:6591-604, which is herein incorporated by reference in its entirety).

In various embodiments, one or more of the following mutations in the Fc region of a fusion molecule described herein may be made: an N297A substitution; an N297Q substitution; an S228P substitution; an L234A substitution; an L234F substitution; an L235A substitution; an L235F substitution; an L235V substitution; an L237A substitution; an S239D substitution; an E233P substitution; an L234V substitution; an L235A substitution; a C236 deletion; a P238A substitution; an S239D substitution; an F243L substitution; a D265A substitution; an S267E substitution; an L328F substitution; an R292P substitution; a Y300L substitution; an A327Q substitution; a P329A substitution; a P329G substitution; an A332L substitution; an 1332E substitution; or a P396L substitution, numbered according to the EU numbering system.

In a specific embodiment, a fusion molecule described herein comprises the Fc region of an IgG1 with an N297Q or N297A amino acid substitution, numbered according to the EU numbering system. In one embodiment, a fusion molecule described herein comprises the Fc region of an IgG1 with a mutation selected from the group consisting of D265A, N297A, and a combination thereof (DANA), numbered according to the EU numbering system. In one embodiment, a fusion molecule described herein comprises the Fc region of an IgG1 with a mutation selected from the group consisting of S239D, 1332E, and a combination thereof (DE), numbered according to the EU numbering system. In one embodiment, a fusion molecule described herein comprises the Fc region of an IgG1 with a mutation selected from the group consisting of D265A, P329A, and a combination thereof, numbered according to the EU numbering system. In another embodiment, a fusion molecule described herein comprises the Fc region of an IgG1 with a mutation selected from the group consisting of L234A, L235A, and a combination thereof (LALA), numbered according to the EU numbering system. In another embodiment, a fusion molecule described herein comprises the Fc region of an IgG1 with a mutation selected from the group consisting of L234A, L235A, P329G, and a combination thereof (LALAPG), numbered according to the EU numbering system. In another embodiment, a fusion molecule described herein comprises the Fc region of an IgG2a with a mutation selected from the group consisting of L234A, L235A, P329G, and a combination thereof (LALAPG), numbered according to the EU numbering system. In another embodiment, a fusion molecule described herein comprises the Fc region of an IgG1 with a mutation selected from the group consisting of L234F, L235F, N297A, and a combination thereof, numbered according to the EU numbering system. In certain embodiments, amino acid residues in the Fc region of a fusion molecule described herein in the positions corresponding to positions L234, L235, and D265 in a human IgG1 heavy chain, numbered according to the EU numbering system, are not L, L, and D, respectively. This approach is described in detail in International Publication No. WO 14/108483, which is herein incorporated by reference in its entirety. In a particular embodiment, the amino acids corresponding to positions L234, L235, and D265 in a human IgG1 Fc region are F, E, and A; or A, A, and A, respectively, numbered according to the EU numbering system. In a particular embodiment, the amino acids corresponding to positions L234, L235, and P329 in a human IgG1 Fc region are A, A, and G, respectively, numbered according to the EU numbering system. In a particular embodiment, the amino acids corresponding to positions D265 and N297 in a human IgG1 Fc region are A and A, respectively, numbered according to the EU numbering system. In a particular embodiment, the amino acids corresponding to positions S239 and 1332 in a human IgG1 Fc region are D and E, respectively, numbered according to the EU numbering system.

In certain embodiments, one or more amino acids selected from amino acid residues 329, 331, and 322 in the Fc region of a fusion molecule described herein, numbered according to the EU numbering system, can be replaced with a different amino acid residue such that the molecule has altered C1q binding and/or reduced or abolished complement dependent cytotoxicity (CDC). This approach is described in further detail in U.S. Pat. No. 6,194,551 (Idusogie et al.), which is herein incorporated by reference in its entirety. In certain embodiments, one or more amino acid residues within amino acid positions 231 to 238 in the N-terminal region of the CH2 domain of an antibody described herein are altered to thereby alter the ability of the molecule to fix complement, numbered according to the EU numbering system. This approach is described further in International Publication No. WO 94/29351, which is herein incorporated by reference in its entirety. In certain embodiments, the Fc region of a fusion molecule described herein is modified to increase the ability of the molecule to mediate antibody dependent cellular cytotoxicity (ADCC) and/or to increase the affinity of the fusion molecule for an Fcγ receptor by mutating one or more amino acids (e.g., introducing amino acid substitutions) at the following positions: 238, 239, 248, 249, 252, 254, 255, 256, 258, 265, 267, 268, 269, 270, 272, 276, 278, 280, 283, 285, 286, 289, 290, 292, 293, 294, 295, 296, 298, 301, 303, 305, 307, 309, 312, 315, 320, 322, 324, 326, 327, 328, 329, 330, 331, 333, 334, 335, 337, 338, 340, 360, 373, 376, 378, 382, 388, 389, 398, 414, 416, 419, 430, 434, 435, 437, 438, or 439, numbered according to the EU numbering system. This approach is described further in International Publication No. WO 00/42072, which is herein incorporated by reference in its entirety.

In certain embodiments, a mutation selected from the group consisting of D265A, P329A, and a combination thereof, numbered according to the EU numbering system, may be made in the Fc region of a fusion molecule described herein. In certain embodiments, a mutation selected from the group consisting of L235A, L237A, and a combination thereof, numbered according to the EU numbering system, may be made in the Fc region of a fusion molecule described herein. In certain embodiments, a mutation selected from the group consisting of S267E, L328F, and a combination thereof, numbered according to the EU numbering system, may be made in the Fc region of a fusion molecule described herein. In certain embodiments, a mutation selected from the group consisting of S239D, 1332E, optionally A330L, and a combination thereof, numbered according to the EU numbering system, may be made in the Fc region of a fusion molecule described herein. In certain embodiments, a mutation selected from the group consisting of L235V, F243L, R292P, Y300L, P396L, and a combination thereof, numbered according to the EU numbering system, may be made in the Fc region of a fusion molecule described herein. In certain embodiments, a mutation selected from the group consisting of S267E, L328F, and a combination thereof, numbered according to the EU numbering system, may be made in the Fc region of a fusion molecule described herein.

In some embodiments, the Fc region of the fusion molecule described herein comprises a methionine (M) to tyrosine (Y) substitution in position 252, a serine(S) to threonine (T) substitution in position 254, and a threonine (T) to glutamic acid (E) substitution in position 256, numbered according to the EU numbering system. See, U.S. Pat. No. 7,658,921, which is herein incorporated by reference in its entirety. This type of mutant IgG, referred to as “YTE mutant” has been shown to display fourfold increased half-life as compared to wild-type versions of the same antibody (see, Dall'Acqua W F et al., (2006) J Biol Chem 281:23514-24, which is herein incorporated by reference in its entirety). In certain embodiments, a fusion molecule comprises an IgG Fc region comprising one or more amino acid substitutions of amino acid residues at positions 251-257, 285-290, 308-314, 385-389, and 428-436, numbered according to the EU numbering system.

In certain embodiments, a fusion molecule described herein comprises the Fc region of human IgG4, and the serine at amino acid residue 228 is substituted for proline (S228P), numbered according to the EU numbering system.

In some embodiments, the Fc region is a human IgG1 Fc, or a derivative thereof. In some embodiments, the human IgG Fc or human IgG1 Fc comprises an amino acid sequence at least 85, 90, 95, 96, 97, 98, or 99% identical to the amino acid sequence of human IgG1 Fc. In some embodiments, the human IgG Fc or IgG1 Fc comprises an amino acid sequence at least 85, 90, 95, 96, 97, 98, 99, or 100% identical to an amino acid sequence provided below in Table 2.

In some embodiments, the Fc region is a human IgG4 Fc, or a derivative thereof. In some embodiments, the human IgG Fc or human IgG4 Fc comprises an amino acid sequence at least 85, 90, 95, 96, 97, 98, or 99% identical to the amino acid sequence of human IgG4 Fc. In some embodiments, the human IgG Fc or IgG4 Fc comprises an amino acid sequence at least 85, 90, 95, 96, 97, 98, 99, or 100% identical to an amino acid sequence provided below in Table 2.

In some embodiments, the Fc region is a mouse IgG2a Fc, or a derivative thereof. In some embodiments, the mouse IgG Fc or mouse IgG2a Fc comprises an amino acid sequence at least 85, 90, 95, 96, 97, 98, or 99% identical to the amino acid sequence of mouse IgG2a Fc. In some embodiments, the mouse IgG Fc or IgG2a Fc comprises an amino acid sequence at least 85, 90, 95, 96, 97, 98, 99, or 100% identical to an amino acid sequence provided below in Table 2.

TABLE 2
Exemplary IgG Fc Amino Acid Sequences

| | | SEQ ID |
|---|---|---|
| Description | Sequence | NO: |
| Human IgG1 Fc | DKTHTCPPCPAPELLGGPSVFLFPPKPKDTLMISRTPEVTCVVVDVSHEDP | 5 |
| | EVKFNWYVDGVEVHNAKTKPREEQYNSTYRVVSVLTVLHQDWLNGKEYKCK | |
| | VSNKALPAPIEKTISKAKGQPREPQVYTLPPSRDELTKNQVSLTCLVKGFY | |
| | PSDIAVEWESNGQPENNYKTTPPVLDSDGSFFLYSKLTVDKSRWQQGNVES | |
| | CSVMHEALHNHYTQKSLSLSPGK | |
| Human IgG1 Fc | DKTHTCPPCPAPELLGGPSVFLFPPKPKDTLMISRTPEVTCVVVDVSHEDP | 6 |
| without C- | EVKENWYVDGVEVHNAKTKPREEQYNSTYRVVSVLTVLHQDWLNGKEYKCK | |
| terminal lysine | VSNKALPAPIEKTISKAKGQPREPQVYTLPPSRDELTKNQVSLTCLVKGFY | |
| | PSDIAVEWESNGQPENNYKTTPPVLDSDGSFFLYSKLTVDKSRWQQGNVES | |
| | CSVMHEALHNHYTQKSLSLSPG | |
| Human IgG1 Fc | DKTHTCPPCPAPELLGGPSVFLFPPKPKDTLMISRTPEVTCVVVAVSHEDP | 7 |
| DANA | EVKENWYVDGVEVHNAKTKPREEQYASTYRVVSVLTVLHQDWLNGKEYKCK | |
| | VSNKALPAPIEKTISKAKGQPREPQVYTLPPSRDELTKNQVSLTCLVKGFY | |
| | PSDIAVEWESNGQPENNYKTTPPVLDSDGSFFLYSKLTVDKSRWQQGNVES | |
| | CSVMHEALHNHYTQKSLSLSPGK | |
| Human IgG1 Fc | DKTHTCPPCPAPELLGGPSVFLFPPKPKDTLMISRTPEVTCVVVAVSHEDP | 8 |
| DANA without | EVKFNWYVDGVEVHNAKTKPREEQYASTYRVVSVLTVLHQDWLNGKEYKCK | |
| C-terminal lysine | VSNKALPAPIEKTISKAKGQPREPQVYTLPPSRDELTKNQVSLTCLVKGFY | |
| | PSDIAVEWESNGQPENNYKTTPPVLDSDGSFFLYSKLTVDKSRWQQGNVFS | |
| | CSVMHEALHNHYTQKSLSLSPG | |
| Human IgG1 Fc | DKTHTCPPCPAPEAAGGPSVELFPPKPKDTLMISRTPEVTCVVVDVSHEDP | 9 |
| LALAPG | EVKFNWYVDGVEVHNAKTKPREEQYNSTYRVVSVLTVLHQDWLNGKEYKCK | |
| | VSNKALGAPIEKTISKAKGQPREPQVYTLPPSRDELTKNQVSLTCLVKGFY | |
| | PSDIAVEWESNGQPENNYKTTPPVLDSDGSFFLYSKLTVDKSRWQQGNVES | |
| | CSVMHEALHNHYTQKSLSLSPGK | |
| Human IgG1 Fc | DKTHTCPPCPAPEAAGGPSVFLFPPKPKDTLMISRTPEVTCVVVDVSHEDP | 10 |
| LALAPG | EVKFNWYVDGVEVHNAKTKPREEQYNSTYRVVSVLTVLHQDWLNGKEYKCK | |
| without C- | VSNKALGAPIEKTISKAKGQPREPQVYTLPPSRDELTKNQVSLTCLVKGFY | |
| terminal lysine | PSDIAVEWESNGQPENNYKTTPPVLDSDGSFFLYSKLTVDKSRWQQGNVES | |
| | CSVMHEALHNHYTQKSLSLSPG | |
| Human IgG1 Fc | DKTHTCPPCPAPELLGGPDVFLFPPKPKDTLMISRTPEVTCVVVDVSHEDP | 11 |
| DE | EVKFNWYVDGVEVHNAKTKPREEQYNSTYRVVSVLTVLHQDWLNGKEYKCK | |
| | VSNKALPAPEEKTISKAKGQPREPQVYTLPPSRDELTKNQVSLTCLVKGFY | |
| | PSDIAVEWESNGQPENNYKTTPPVLDSDGSFFLYSKLTVDKSRWQQGNVES | |
| | CSVMHEALHNHYTQKSLSLSPGK | |
| Human IgG1 Fc | DKTHTCPPCPAPELLGGPDVFLFPPKPKDTLMISRTPEVTCVVVDVSHEDP | 12 |
| DE without C- | EVKFNWYVDGVEVHNAKTKPREEQYNSTYRVVSVLTVLHQDWLNGKEYKCK | |
| terminal lysine | VSNKALPAPEEKTISKAKGQPREPQVYTLPPSRDELTKNQVSLTCLVKGFY | |
| | PSDIAVEWESNGQPENNYKTTPPVLDSDGSFFLYSKLTVDKSRWQQGNVES | |
| | CSVMHEALHNHYTQKSLSLSPG | |
| Human IgG4 Fc | ESKYGPPCPPCPAPEFLGGPSVFLFPPKPKDTLMISRTPEVTCVVVDVSQE | 13 |
| S228P | DPEVQFNWYVDGVEVHNAKTKPREEQFNSTYRVVSVLTVLHQDWLNGKEYK | |
| | CKVSNKGLPSSIEKTISKAKGQPREPQVYTLPPSQEEMTKNQVSLTCLVKG | |
| | FYPSDIAVEWESNGQPENNYKTTPPVLDSDGSFFLYSRLTVDKSRWQEGNV | |
| | FSCSVMHEALHNHYTQKSLSLSLGK | |
| Human IgG4 Fc | ESKYGPPCPPCPAPEFLGGPSVFLFPPKPKDTLMISRTPEVTCVVVDVSQE | 14 |
| S228P without | DPEVQFNWYVDGVEVHNAKTKPREEQFNSTYRVVSVLTVLHQDWLNGKEYK | |
| C-terminal lysine | CKVSNKGLPSSIEKTISKAKGQPREPQVYTLPPSQEEMTKNQVSLTCLVKG | |
| | FYPSDIAVEWESNGQPENNYKTTPPVLDSDGSFFLYSRLTVDKSRWQEGNV | |
| | FSCSVMHEALHNHYTQKSLSLSLG | |
| Mouse IgG2a Fc | EPRGPTIKPCPPCKCPAPNLLGGPSVFIFPPKIKDVLMISLSPIVTCVVVD | 15 |
| | VSEDDPDVQISWFVNNVEVHTAQTQTHREDYNSTLRVVSALPIQHQDWMSG | |
| | KEFKCKVNNKDLPAPIERTISKPKGSVRAPQVYVLPPPEEEMTKKQVTLTC | |
| | MVTDEMPEDIYVEWTNNGKTELNYKNTEPVLDSDGSYFMYSKLRVEKKNWV | |
| | ERNSYSCSVVHEGLHNHHTTKSFSRTPGK | |
| Mouse IgG2a Fc | EPRGPTIKPCPPCKCPAPNLLGGPSVFIFPPKIKDVLMISLSPIVTCVVVD | 16 |
| without C- | VSEDDPDVQISWFVNNVEVHTAQTQTHREDYNSTLRVVSALPIQHQDWMSG | |
| terminal lysine | KEFKCKVNNKDLPAPIERTISKPKGSVRAPQVYVLPPPEEEMTKKQVTLTC | |
| | MVTDEMPEDIYVEWTNNGKTELNYKNTEPVLDSDGSYFMYSKLRVEKKNWV | |
| | ERNSYSCSVVHEGLHNHHTTKSFSRTPG | |
| Mouse IgG2a Fc | EPRGPTIKPCPPCKCPAPNAAGGPSVFIFPPKIKDVLMISLSPIVTCVVVD | 17 |
| LALAPG | VSEDDPDVQISWFVNNVEVHTAQTQTHREDYNSTLRVVSALPIQHQDWMSG | |
| | KEFKCKVNNKDLGAPIERTISKPKGSVRAPQVYVLPPPEEEMTKKQVTLTC | |
| | MVTDEMPEDIYVEWTNNGKTELNYKNTEPVLDSDGSYFMYSKLRVEKKNWV | |
| | ERNSYSCSVVHEGLHNHHTTKSFSRTPGK | |
| Mouse IgG2a Fc | EPRGPTIKPCPPCKCPAPNAAGGPSVFIFPPKIKDVLMISLSPIVTCVVVD | 18 |
| LALAPG | VSEDDPDVQISWFVNNVEVHTAQTQTHREDYNSTLRVVSALPIQHQDWMSG | |
| without C- | KEFKCKVNNKDLGAPIERTISKPKGSVRAPQVYVLPPPEEEMTKKQVTLTC | |
| terminal lysine | MVTDEMPEDIYVEWTNNGKTELNYKNTEPVLDSDGSYFMYSKLRVEKKNWV | |
| | ERNSYSCSVVHEGLHNHHTTKSFSRTPG | |
| Human IgG1 Fc | EPKSXDKTHTCPPCPAPELLGGPSVFLFPPKPKDTLMISRTPEVTCVVVDV | 50 |
| (including N- | SHEDPEVKFNWYVDGVEVHNAKTKPREEQYNSTYRVVSVLTVLHQDWLNGK | |
| terminal five | EYKCKVSNKALPAPIEKTISKAKGQPREPQVYTLPPSRDELTKNQVSLTCL | |
| hinge residues) | VKGFYPSDIAVEWESNGQPENNYKTTPPVLDSDGSFFLYSKLTVDKSRWQQ | |
| | GNVFSCSVMHEALHNHYTQKSLSLSPGK(whereinXisCQrS) | |
| Human IgG1 Fc | EPKSXDKTHTCPPCPAPELLGGPSVFLFPPKPKDTLMISRTPEVTCVVVDV | 51 |
| (including N- | SHEDPEVKFNWYVDGVEVHNAKTKPREEQYNSTYRVVSVLTVLHQDWLNGK | |
| terminal five | EYKCKVSNKALPAPIEKTISKAKGQPREPQVYTLPPSRDELTKNQVSLTCL | |
| hinge residues) | VKGFYPSDIAVEWESNGQPENNYKTTPPVLDSDGSFFLYSKLTVDKSRWQQ | |
| without C- | GNVFSCSVMHEALHNHYTQKSLSLSPG(whereinXisCQrS) | |
| terminal lysine | | |
| Human IgG1 Fc | CPPCPAPELLGGPSVFLFPPKPKDTLMISRTPEVTCVVVDVSHEDPEVKEN | 52 |
| (excluding upper | WYVDGVEVHNAKTKPREEQYNSTYRVVSVLTVLHQDWLNGKEYKCKVSNKA | |
| hinge region) | LPAPIEKTISKAKGQPREPQVYTLPPSRDELTKNQVSLTCLVKGFYPSDIA | |
| | VEWESNGQPENNYKTTPPVLDSDGSFFLYSKLTVDKSRWQQGNVFSCSVMH | |
| | EALHNHYTQKSLSLSPGK | |
| Human IgG1 Fc | CPPCPAPELLGGPSVFLFPPKPKDTLMISRTPEVTCVVVDVSHEDPEVKEN | 53 |
| (excluding upper | WYVDGVEVHNAKTKPREEQYNSTYRVVSVLTVLHQDWLNGKEYKCKVSNKA | |
| hinge region) | LPAPIEKTISKAKGQPREPQVYTLPPSRDELTKNQVSLTCLVKGFYPSDIA | |
| without C- | VEWESNGQPENNYKTTPPVLDSDGSFFLYSKLTVDKSRWQQGNVFSCSVMH | |
| terminal lysine | EALHNHYTQKSLSLSPG | |
| Human IgG1 Fc | APELLGGPSVFLFPPKPKDTLMISRTPEVTCVVVDVSHEDPEVKENWYVDG | 54 |
| (excluding upper | VEVHNAKTKPREEQYNSTYRVVSVLTVLHQDWLNGKEYKCKVSNKALPAPI | |
| and core hinge | EKTISKAKGQPREPQVYTLPPSRDELTKNQVSLTCLVKGFYPSDIAVEWES | |
| regions) | NGQPENNYKTTPPVLDSDGSFFLYSKLTVDKSRWQQGNVFSCSVMHEALHN | |
| | HYTQKSLSLSPGK | |
| Human IgG1 Fc | APELLGGPSVFLFPPKPKDTLMISRTPEVTCVVVDVSHEDPEVKENWYVDG | 55 |
| (excluding upper | VEVHNAKTKPREEQYNSTYRVVSVLTVLHQDWLNGKEYKCKVSNKALPAPI | |
| and core hinge | EKTISKAKGQPREPQVYTLPPSRDELTKNQVSLTCLVKGFYPSDIAVEWES | |
| regions) | NGQPENNYKTTPPVLDSDGSFFLYSKLTVDKSRWQQGNVFSCSVMHEALHN | |
| without C- | HYTQKSLSLSPG | |
| terminal lysine | | |
| Human IgG1 Fc | SVFLFPPKPKDTLMISRTPEVTCVVVDVSHEDPEVKENWYVDGVEVHNAKT | 56 |
| (excluding upper, | KPREEQYNSTYRVVSVLTVLHQDWLNGKEYKCKVSNKALPAPIEKTISKAK | |
| core, and lower | GQPREPQVYTLPPSRDELTKNQVSLTCLVKGFYPSDIAVEWESNGQPENNY | |
| hinge regions) | KTTPPVLDSDGSFFLYSKLTVDKSRWQQGNVESCSVMHEALHNHYTQKSLS | |
| | LSPGK | |
| Human IgG1 Fc | SVELFPPKPKDTLMISRTPEVTCVVVDVSHEDPEVKENWYVDGVEVHNAKT | 57 |
| (excluding upper, | KPREEQYNSTYRVVSVLTVLHQDWLNGKEYKCKVSNKALPAPIEKTISKAK | |
| core, and lower | GQPREPQVYTLPPSRDELTKNQVSLTCLVKGFYPSDIAVEWESNGQPENNY | |
| hinge regions) | KTTPPVLDSDGSFFLYSKLTVDKSRWQQGNVFSCSVMHEALHNHYTQKSLS | |
| without C- | LSPG | |
| terminal lysine | | |

In some embodiments, any IgG Fc, or derivative thereof, can be linked to the N-terminus or C-terminus of any of the SMAGP ECDs described in Table 1 above with or without a linker. In some embodiments, human IgG1 Fc, or a derivative thereof, can be linked to the N-terminus or C-terminus of any of SMAGP ECDs described in Table 1 above with or without a linker. In some embodiments, the amino acid sequence of the human IgG1 Fc comprises or consists of an amino acid sequence selected from the group consisting of SEQ ID NO: 5, 6, and 50-57. In some embodiments, the derivative of the human IgG1 Fc comprises an amino acid sequence at least 85, 90, 95, 96, 97, 98, or 99% identical to an amino acid sequence selected from the group consisting of SEQ ID NO: 5, 6, and 50-57.

In some embodiments, a human IgG1 Fc comprising a DANA mutation, or a derivative thereof, can be linked to the N-terminus or C-terminus of any of the SMAGP ECDs described in Table 1 above with or without a linker. In some embodiments, the amino acid sequence of the human IgG1 Fc comprising a DANA mutation comprises or consists of the amino acid sequence of SEQ ID NO: 7 or 8. In some embodiments, the derivative of the human IgG1 Fc comprising a DANA mutation comprises an amino acid sequence at least 85, 90, 95, 96, 97, 98, or 99% identical to the amino acid sequence of SEQ ID NO: 7 or 8.

In some embodiments, a human IgG1 Fc comprising a LALAPG mutation, or a derivative thereof, can be linked to the N-terminus or C-terminus of any of the SMAGP ECDs described in Table 1 above with or without a linker. In some embodiments, the amino acid sequence of the human IgG1 Fc comprising a LALAPG mutation comprises or consists of the amino acid sequence of SEQ ID NO: 9 or 10. In some embodiments, the derivative of the human IgG1 Fc comprising a LALAPG mutation comprises an amino acid sequence at least 85, 90, 95, 96, 97, 98, or 99% identical to the amino acid sequence of SEQ ID NO: 9 or 10.

In some embodiments, a human IgG1 Fc comprising a DE mutation, or a derivative thereof, can be linked to the N-terminus or C-terminus of any of the SMAGP ECDs described in Table 1 above with or without a linker. In some embodiments, the amino acid sequence of the human IgG1 Fc comprising a DE mutation comprises or consists of the amino acid sequence of SEQ ID NO: 11 or 12. In some embodiments, the derivative of the human IgG1 Fc comprising a DE mutation comprises an amino acid sequence at least 85, 90, 95, 96, 97, 98, or 99% identical to the amino acid sequence of SEQ ID NO: 11 or 12.

In some embodiments, a human IgG4 Fc comprising a S228P mutation, or a derivative thereof, can be linked to the N-terminus or C-terminus of any of the SMAGP ECDs described in Table 1 above with or without a linker. In some embodiments, the amino acid sequence of the human IgG4 Fc comprising a S228P mutation comprises or consists of the amino acid sequence of SEQ ID NO: 13 or 14. In some embodiments, the derivative of the human IgG4 Fc comprising a S228P mutation comprises an amino acid sequence at least 85, 90, 95, 96, 97, 98, or 99% identical to the amino acid sequence of SEQ ID NO: 13 or 14.

In some embodiments, a mouse IgG2a Fc, or a derivative thereof, can be linked to the N-terminus or C-terminus of any of the SMAGP ECDs described in Table 1 above with or without a linker. In some embodiments, the amino acid sequence of the mouse IgG2a Fc comprises or consists of the amino acid sequence of SEQ ID NO: 15 or 16. In some embodiments, the derivative of the mouse IgG2a Fc comprises an amino acid sequence at least 85, 90, 95, 96, 97, 98, or 99% identical to the amino acid sequence of SEQ ID NO: 15 or 16.

In some embodiments, a mouse IgG2a Fc comprising a LALAPG mutation, or a derivative thereof, can be linked to the N-terminus or C-terminus of any of the SMAGP ECDs described in Table 1 above with or without a linker. In some embodiments, the amino acid sequence of the mouse IgG2a Fc comprising a LALAPG mutation comprises or consists of the amino acid sequence of SEQ ID NO: 17 or 18. In some embodiments, the derivative of the mouse IgG2a Fc comprising a LALAPG mutation comprises an amino acid sequence at least 85, 90, 95, 96, 97, 98, or 99% identical to the amino acid sequence of SEQ ID NO: 17 or 18.

In some embodiments, the fusion molecule comprises or consists of the amino acid sequences shown in Table 3, below.

TABLE 3
Exemplary Fusion Molecule Amino Acid Sequences

| | | SEQ ID |
|---|---|---|
| Description | Sequence | NO: |
| HuSMAGP- | MTSLLTTPSPREELMTTPILQPTEALSPEDGASTALEPKSSDKTHTCPPCPA | 19 |
| hIgG1 | PELLGGPSVFLFPPKPKDTLMISRTPEVTCVVVDVSHEDPEVKENWYVDGVE | |
| | VHNAKTKPREEQYNSTYRVVSVLTVLHQDWLNGKEYKCKVSNKALPAPIEKT | |
| | ISKAKGQPREPQVYTLPPSRDELTKNQVSLTCLVKGFYPSDIAVEWESNGQP | |
| | ENNYKTTPPVLDSDGSFFLYSKLTVDKSRWQQGNVFSCSVMHEALHNHYTQK | |
| | SLSLSPGK | |
| HuSMAGP- | MTSLLTTPSPREELMTTPILQPTEALSPEDGASTALEPKSSDKTHTCPPCPA | 20 |
| hIgG1 without | PELLGGPSVELFPPKPKDTLMISRTPEVTCVVVDVSHEDPEVKENWYVDGVE | |
| C-terminal | VHNAKTKPREEQYNSTYRVVSVLTVLHQDWLNGKEYKCKVSNKALPAPIEKT | |
| lysine | ISKAKGQPREPQVYTLPPSRDELTKNQVSLTCLVKGFYPSDIAVEWESNGQP | |
| | ENNYKTTPPVLDSDGSFFLYSKLTVDKSRWQQGNVFSCSVMHEALHNHYTQK | |
| | SLSLSPG | |
| HuSMAGP- | MTSLLTTPSPREELMTTPILQPTEALSPEDGASTALEPKSSDKTHTCPPCPA | 21 |
| hIgG1-DANA | PELLGGPSVFLFPPKPKDTLMISRTPEVTCVVVAVSHEDPEVKENWYVDGVE | |
| | VHNAKTKPREEQYASTYRVVSVLTVLHQDWLNGKEYKCKVSNKALPAPIEKT | |
| | ISKAKGQPREPQVYTLPPSRDELTKNQVSLTCLVKGFYPSDIAVEWESNGQP | |
| | ENNYKTTPPVLDSDGSFFLYSKLTVDKSRWQQGNVFSCSVMHEALHNHYTQK | |
| | SLSLSPGK | |
| HuSMAGP- | MTSLLTTPSPREELMTTPILQPTEALSPEDGASTALEPKSSDKTHTCPPCPA | 22 |
| hIgG1-DANA | PELLGGPSVFLFPPKPKDTLMISRTPEVTCVVVAVSHEDPEVKENWYVDGVE | |
| without C- | VHNAKTKPREEQYASTYRVVSVLTVLHQDWLNGKEYKCKVSNKALPAPIEKT | |
| terminal lysine | ISKAKGQPREPQVYTLPPSRDELTKNQVSLTCLVKGFYPSDIAVEWESNGQP | |
| | ENNYKTTPPVLDSDGSFFLYSKLTVDKSRWQQGNVFSCSVMHEALHNHYTQK | |
| | SLSLSPG | |
| HuSMAGP- | MTSLLTTPSPREELMTTPILQPTEALSPEDGASTALEPKSSDKTHTCPPCPA | 23 |
| hIgG1- | PEAAGGPSVFLFPPKPKDTLMISRTPEVTCVVVDVSHEDPEVKENWYVDGVE | |
| LALAPG | VHNAKTKPREEQYNSTYRVVSVLTVLHQDWLNGKEYKCKVSNKALGAPIEKT | |
| | ISKAKGQPREPQVYTLPPSRDELTKNQVSLTCLVKGFYPSDIAVEWESNGQP | |
| | ENNYKTTPPVLDSDGSFFLYSKLTVDKSRWQQGNVFSCSVMHEALHNHYTQK | |
| | SLSLSPGK | |
| HuSMAGP- | MTSLLTTPSPREELMTTPILQPTEALSPEDGASTALEPKSSDKTHTCPPCPA | 24 |
| hIgG1- | PEAAGGPSVFLFPPKPKDTLMISRTPEVTCVVVDVSHEDPEVKENWYVDGVE | |
| LALAPG | VHNAKTKPREEQYNSTYRVVSVLTVLHQDWLNGKEYKCKVSNKALGAPIEKT | |
| without C- | ISKAKGQPREPQVYTLPPSRDELTKNQVSLTCLVKGFYPSDIAVEWESNGQP | |
| terminal lysine | ENNYKTTPPVLDSDGSFFLYSKLTVDKSRWQQGNVFSCSVMHEALHNHYTQK | |
| | SLSLSPG | |
| HuSMAGP- | MTSLLTTPSPREELMTTPILQPTEALSPEDGASTALEPKSSDKTHTCPPCPA | 25 |
| hIgG1-DE | PELLGGPDVFLFPPKPKDTLMISRTPEVTCVVVDVSHEDPEVKENWYVDGVE | |
| | VHNAKTKPREEQYNSTYRVVSVLTVLHQDWLNGKEYKCKVSNKALPAPEEKT | |
| | ISKAKGQPREPQVYTLPPSRDELTKNQVSLTCLVKGFYPSDIAVEWESNGQP | |
| | ENNYKTTPPVLDSDGSFFLYSKLTVDKSRWQQGNVFSCSVMHEALHNHYTQK | |
| | SLSLSPGK | |
| HuSMAGP- | MTSLLTTPSPREELMTTPILQPTEALSPEDGASTALEPKSSDKTHTCPPCPA | 26 |
| hIgG1-DE | PELLGGPDVFLFPPKPKDTLMISRTPEVTCVVVDVSHEDPEVKENWYVDGVE | |
| without C- | VHNAKTKPREEQYNSTYRVVSVLTVLHQDWLNGKEYKCKVSNKALPAPEEKT | |
| terminal lysine | ISKAKGQPREPQVYTLPPSRDELTKNQVSLTCLVKGFYPSDIAVEWESNGQP | |
| | ENNYKTTPPVLDSDGSFFLYSKLTVDKSRWQQGNVFSCSVMHEALHNHYTQK | |
| | SLSLSPG | |
| HuSMAGP- | MTSLLTTPSPREELMTTPILQPTEALSPEDGASTALESKYGPPCPPCPAPEF | 27 |
| hIgG4 | LGGPSVFLFPPKPKDTLMISRTPEVTCVVVDVSQEDPEVQFNWYVDGVEVHN | |
| | AKTKPREEQFNSTYRVVSVLTVLHQDWLNGKEYKCKVSNKGLPSSIEKTISK | |
| | AKGQPREPQVYTLPPSQEEMTKNQVSLTCLVKGFYPSDIAVEWESNGQPENN | |
| | YKTTPPVLDSDGSFFLYSRLTVDKSRWQEGNVFSCSVMHEALHNHYTQKSLS | |
| | LSLGK | |
| HuSMAGP- | MTSLLTTPSPREELMTTPILQPTEALSPEDGASTALESKYGPPCPPCPAPEF | 28 |
| hIgG4 without | LGGPSVFLFPPKPKDTLMISRTPEVTCVVVDVSQEDPEVQFNWYVDGVEVHN | |
| C-terminal | AKTKPREEQFNSTYRVVSVLTVLHQDWLNGKEYKCKVSNKGLPSSIEKTISK | |
| lysine | AKGQPREPQVYTLPPSQEEMTKNQVSLTCLVKGFYPSDIAVEWESNGQPENN | |
| | YKTTPPVLDSDGSFFLYSRLTVDKSRWQEGNVFSCSVMHEALHNHYTQKSLS | |
| | LSLG | |
| MuSMAGP- | MNNLPATPSPEELMTTPVFQAPETLSPQAEEASTALEPRGPTIKPCPPCKCP | 29 |
| mIgG2a | APNLLGGPSVFIFPPKIKDVLMISLSPIVTCVVVDVSEDDPDVQISWFVNNV | |
| | EVHTAQTQTHREDYNSTLRVVSALPIQHQDWMSGKEFKCKVNNKDLPAPIER | |
| | TISKPKGSVRAPQVYVLPPPEEEMTKKQVTLTCMVTDEMPEDIYVEWTNNGK | |
| | TELNYKNTEPVLDSDGSYFMYSKLRVEKKNWVERNSYSCSVVHEGLHNHHTT | |
| | KSFSRTPGK | |
| MuSMAGP- | MNNLPATPSPEELMTTPVFQAPETLSPQAEEASTALEPRGPTIKPCPPCKCP | 30 |
| mIgG2a without | APNLLGGPSVFIFPPKIKDVLMISLSPIVTCVVVDVSEDDPDVQISWFVNNV | |
| C-terminal | EVHTAQTQTHREDYNSTLRVVSALPIQHQDWMSGKEFKCKVNNKDLPAPIER | |
| lysine | TISKPKGSVRAPQVYVLPPPEEEMTKKQVTLTCMVTDEMPEDIYVEWTNNGK | |
| | TELNYKNTEPVLDSDGSYFMYSKLRVEKKNWVERNSYSCSVVHEGLHNHHTT | |
| | KSFSRTPG | |
| MuSMAGP- | MNNLPATPSPEELMTTPVFQAPETLSPQAEEASTALEPRGPTIKPCPPCKCP | 31 |
| mIgG2a- | APNAAGGPSVFIFPPKIKDVLMISLSPIVTCVVVDVSEDDPDVQISWFVNNV | |
| LALAPG | EVHTAQTQTHREDYNSTLRVVSALPIQHQDWMSGKEFKCKVNNKDLGAPIER | |
| | TISKPKGSVRAPQVYVLPPPEEEMTKKQVTLTCMVTDEMPEDIYVEWTNNGK | |
| | TELNYKNTEPVLDSDGSYFMYSKLRVEKKNWVERNSYSCSVVHEGLHNHHTT | |
| | KSFSRTPGK | |
| MuSMAGP- | MNNLPATPSPEELMTTPVFQAPETLSPQAEEASTALEPRGPTIKPCPPCKCP | 32 |
| mIgG2a- | APNAAGGPSVFIFPPKIKDVLMISLSPIVTCVVVDVSEDDPDVQISWFVNNV | |
| LALAPG | EVHTAQTQTHREDYNSTLRVVSALPIQHQDWMSGKEFKCKVNNKDLGAPIER | |
| without C- | TISKPKGSVRAPQVYVLPPPEEEMTKKQVTLTCMVTDEMPEDIYVEWTNNGK | |
| terminal lysine | TELNYKNTEPVLDSDGSYFMYSKLRVEKKNWVERNSYSCSVVHEGLHNHHTT | |
| | KSFSRTPG | |
| CySMAGP- | MTSLLTTPSPRELMTTPILRPTEALSPEDGASTALEPKSSDKTHTCPPCPAP | 33 |
| hIgG1 | ELLGGPSVFLFPPKPKDTLMISRTPEVTCVVVDVSHEDPEVKENWYVDGVEV | |
| | HNAKTKPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVEWESNGQPE | |
| | NNYKTTPPVLDSDGSFFLYSKLTVDKSRWQQGNVFSCSVMHEALHNHYTQKS | |
| | LSLSPGK | |
| CySMAGP- | MTSLLTTPSPRELMTTPILRPTEALSPEDGASTALEPKSSDKTHTCPPCPAP | 34 |
| hIgG1 without | ELLGGPSVFLFPPKPKDTLMISRTPEVTCVVVDVSHEDPEVKENWYVDGVEV | |
| C-terminal | HNAKTKPREEQYNSTYRVVSVLTVLHQDWLNGKEYKCKVSNKALPAPIEKTI | |
| lysine | SKAKGQPREPQVYTLPPSRDELTKNQVSLTCLVKGFYPSDIAVEWESNGQPE | |
| | NNYKTTPPVLDSDGSFFLYSKLTVDKSRWQQGNVFSCSVMHEALHNHYTQKS | |
| | LSLSPG | |

In some embodiments, the disclosure provides a fusion molecule comprising an amino acid sequence at least 85, 90, 95, 96, 97, 98, or 99% identical to the amino acid sequence of any one of SEQ ID NOs: 19-34. In some embodiments, the disclosure provides a fusion molecule comprising the amino acid sequence of any one of SEQ ID NOs: 19-34. In some embodiments, the disclosure provides a fusion molecule consisting of the amino acid sequence of any one of SEQ ID NOs: 19-34.

In some embodiments, the disclosure provides a fusion molecule comprising a dimer of two polypeptides, each polypeptide comprising an amino acid sequence at least 85, 90, 95, 96, 97, 98, or 99% identical to the amino acid sequence of any one of SEQ ID NOs: 19-34. In some embodiments, the disclosure provides a fusion molecule comprising a dimer of two polypeptides, each polypeptide comprising an amino acid sequence of any one of SEQ ID NOs: 19-34. In some embodiments, the disclosure provides a fusion molecule comprising a dimer of two polypeptides, each polypeptide consisting of an amino acid sequence of any one of SEQ ID NOs: 19-34. In some embodiments, the disclosure provides a fusion molecule consisting of a dimer of two polypeptides, each polypeptide consisting of an amino acid sequence of any one of SEQ ID NOs: 19-34.

In some embodiments, the half-life extending moiety is a serum albumin (e.g., human serum albumin) or a derivative thereof.

In some embodiments, the half-life extending moiety is a serum albumin-binding moiety. Any moiety that can specifically bind to serum albumin (e.g., human serum albumin) in a physiological setting can be used in the fusion molecules described herein. Suitable serum albumin-binding moieties include, without limitation, antibodies or antigen-binding portions thereof, and non-antibody binding molecules such as avimers, DARPins, affibodies, and lipocalins. In some embodiments, the albumin-binding moiety is a VH or VHH that specially binds to human serum albumin.

In some embodiments, the half-life extending moiety is a non-proteinaceous molecule, such as a polymer. In some embodiments, the non-proteinaceous molecule is linked to the fusion molecules instead of an Fc region. In some embodiments, the non-proteinaceous molecule is linked to the fusion molecules in addition to an Fc region.

Examples of non-proteinaceous half-life extending moieties include polymer molecules selected from the group consisting of polyalkylene oxide (PAO), including polyalkylene glycol (PAG), such as polyethylene glycol (PEG) and polypropylene glycol (PPG), branched PEGs, hydroxyalkyl starch (HAS), such as hydroxyethyl starch (HES), polysialic acid (PSA), poly-vinyl alcohol (PVA), poly-carboxylate, poly-(vinylpyrrolidone), polyethylene-co-maleic acid anhydride, polystyrene-co-maleic acid anhydride, dextran, including carboxymethyl-dextran, or any other biopolymer suitable for reducing immunogenicity and/or increasing functional in vivo half-life and/or serum half-life. Another example of a polymer molecule is human serum albumin or another abundant plasma protein. Generally, polyalkylene glycol-derived polymers are biocompatible, non-toxic, non-antigenic, non-immunogenic, have various water solubility properties, and are easily excreted from living organisms.

PEG has the advantage of having only few reactive groups capable of cross-linking compared to, e.g., polysaccharides such as dextran. In particular, monofunctional PEG, e.g., methoxypolyethylene glycol (mPEG), is of interest since its coupling chemistry is relatively simple (only one reactive group is available for conjugating with attachment groups on the polypeptide). Consequently, as the risk of cross-linking is eliminated, the resulting conjugated fusion molecules described herein are more homogeneous, and the reaction of the polymer molecules with the variant polypeptide is easier to control.

To effect covalent attachment of the polymer molecule(s) to the fusion molecules described herein, the hydroxyl end groups of the polymer molecule must be provided in activated form, i.e., with reactive functional groups (examples of which include primary amino groups, hydrazide (HZ), thiol, succinate (SUC), succinimidyl succinate (SS), succinimidyl succinamide (SSA), succinimidyl propionate (SPA), succinimidyl butyrate (SBA), succinimidyl carboxymethylate (SCM), benzotriazole carbonate (BTC), N-hydroxysuccinimide (NHS), aldehyde, nitrophenylcarbonate (NPC), and tresylate (TRES)). Suitable activated polymer molecules are commercially available, e.g., from Shearwater Polymers, Inc., Huntsville, Ala., USA, or from PolyMASC Pharmaceuticals plc, UK.

Alternatively, the polymer molecules can be activated by conventional methods known in the art, e.g., as disclosed in WO 90/13540. Specific examples of activated linear or branched polymer molecules for use herein are described in the Shearwater Polymers, Inc. 1997 and 2000 Catalogs (Functionalized Biocompatible Polymers for Research and pharmaceuticals, Polyethylene Glycol and Derivatives, incorporated herein by reference). Specific examples of activated PEG polymers include the following linear PEGs: NHS-PEG (e.g., SPA-PEG, SSPA-PEG, SBA-PEG, SS-PEG, SSA-PEG, SC-PEG, SG-PEG, and SCM-PEG), and NOR-PEG, BTC-PEG, EPOXPEG, NCO-PEG, NPC-PEG, CDI-PEG, ALD-PEG, TRES-PEG, VS-PEG, IODO-PEG, and MAL-PEG, and branched PEGs such as PEG2-NHS and those disclosed in U.S. Pat. Nos. 5,932,462 and 5,643,575, both of which are incorporated herein by reference. Furthermore, the following publications disclose useful polymer molecules and/or PEGylation chemistries: U.S. Pat. Nos. 5,824,778, 5,476,653, WO 97/32607, EP 229,108, EP 402,378, U.S. Pat. Nos. 4,902,502, 5,281,698, 5,122,614, 5,219,564, WO 92/16555, WO 94/04193, WO 94/14758, WO 94/17039, WO 94/18247, WO 94/28024, WO 95/00162, WO 95/11924, WO 95/13090, WO 95/33490, WO 96/00080, WO 97/18832, WO 98/41562, WO 98/48837, WO 99/32134, WO 99/32139, WO 99/32140, WO 96/40791, WO 98/32466, WO 95/06058, EP 439 508, WO 97/03106, WO 96/21469, WO 95/13312, EP 921 131, U.S. Pat. No. 5,736,625, WO 98/05363, EP 809 996, U.S. Pat. No. 5,629,384, WO 96/41813, WO 96/07670, U.S. Pat. Nos. 5,473,034, 5,516,673, EP 605 963, U.S. Pat. No. 5,382,657, EP 510 356, EP 400 472, EP 183 503, and EP 154 316.

Specific examples of activated PEG polymers particularly preferred for coupling to cysteine residues, include the following linear PEGs: vinylsulfone-PEG (VS-PEG), preferably vinylsulfone-mPEG (VS-mPEG); maleimide-PEG (MAL-PEG), preferably maleimide-mPEG (MAL-mPEG) and orthopyridyl-disulfide-PEG (OPSS-PEG), preferably orthopyridyl-disulfide-mPEG (OPSS-mPEG). Typically, such PEG or mPEG polymers will have a size of about 5 kDa, about 10 kDa, about 12 kDa or about 20 kDa.

The conjugation of the fusion molecules described herein and the activated polymer molecules is conducted by use of any conventional method, e.g., as described in the following references (which also describe suitable methods for activation of polymer molecules): Harris and Zalipsky, eds., Poly(ethylene glycol) Chemistry and Biological Applications, AZC Washington; R. F. Taylor, (1991), “Protein immobilisation. Fundamental and applications,” Marcel Dekker, N.Y.; S. S. Wong, (1992), “Chemistry of Protein Conjugation and Crosslinking,” CRC Press, Boca Raton; G. T. Hermanson et al., (1993), “Immobilized Affinity Ligand Techniques”, Academic Press, N.Y.

The skilled person will be aware that the activation method and/or conjugation chemistry to be used depends on the attachment group(s) of the fusion protein (examples of which are given further above), as well as the functional groups of the polymer (e.g., being amine, hydroxyl, carboxyl, aldehyde, sulfhydryl, succinimidyl, maleimide, vinylsulfone or haloacetate). The PEGylation may be directed towards conjugation to all available attachment groups on the fusion molecule (i.e., such attachment groups that are exposed at the surface of the polypeptide) or may be directed towards one or more specific attachment groups, e.g., the N-terminal amino group as described in U.S. Pat. No. 5,985,265 or to cysteine residues. Furthermore, the conjugation may be achieved in one step or in a stepwise manner (e.g., as described in WO 99/55377).

For PEGylation to cysteine residues (see above) the fusion protein is usually treated with a reducing agent, such as dithiothreitol (DDT) prior to PEGylation. The reducing agent is subsequently removed by any conventional method, such as by desalting. Conjugation of PEG to a cysteine residue typically takes place in a suitable buffer at pH 6-9 at temperatures varying from 4° C. to 25° C. for periods up to 16 hours.

It will be understood that the PEGylation is designed so as to produce the optimal molecule with respect to the number of PEG molecules attached, the size and form of such molecules (e.g., whether they are linear or branched), and the attachment site(s) in the fusion molecule. The molecular weight of the polymer to be used may, e.g., be chosen on the basis of the desired effect to be achieved.

In connection with conjugation to only a single attachment group on the fusion molecule (e.g., the N-terminal amino group), it may be advantageous that the polymer molecule, which may be linear or branched, has a high molecular weight, preferably about 10-25 kDa, such as about 15-25 kDa, e.g., about 20 kDa.

Normally, the polymer conjugation is performed under conditions aimed at reacting as many of the available polymer attachment groups with polymer molecules. This is achieved by means of a suitable molar excess of the polymer relative to the polypeptide. Typically, the molar ratios of activated polymer molecules to polypeptide are up to about 1000-1, such as up to about 200-1, or up to about 100-1. In some cases, the ratio may be somewhat lower, however, such as up to about 50-1, 10-1, 5-1, 2-1 or 1-1 in order to obtain optimal reaction.

It is also contemplated to couple the polymer molecules to the fusion molecule through a linker. Suitable linkers are well known to the skilled person. A preferred example is cyanuric chloride (Abuchowski et al., (1977), J. Biol. Chem., 252, 3578-3581; U.S. Pat. No. 4,179,337; Shafer et al., (1986), J. Polym. Sci. Polym. Chem. Ed., 24, 375-378).

Subsequent to the conjugation, residual activated polymer molecules are blocked according to methods known in the art, e.g., by addition of primary amine to the reaction mixture, and the resulting inactivated polymer molecules are removed by a suitable method.

It will be understood that depending on the circumstances, e.g., the amino acid sequence of the fusion molecule, the nature of the activated PEG compound being used, and the specific PEGylation conditions, including the molar ratio of PEG to polypeptide, varying degrees of PEGylation may be obtained, with a higher degree of PEGylation generally being obtained with a higher ratio of PEG to fusion protein. The PEGylated fusion proteins resulting from any given PEGylation process will, however, normally comprise a stochastic distribution of conjugated fusion protein having slightly different degrees of PEGylation.

For improvement of the biological half-life of the fusion proteins described herein, chemical modification such as PEGylation, or HESylation are applicable.

HAS and HES non-proteinaceous polymers, as well as methods of producing HAS or HES conjugates are disclosed for example in WO 02/080979, WO 03/070772, WO 057092391, and WO 057092390.

Polysialylation is another technology, which uses the natural polymer polysialic acid (PSA) to prolong the half-life and improve the stability of therapeutic peptides and proteins. PSA is a polymer of sialic acid (a sugar). When used for protein and therapeutic peptide drug delivery, polysialic acid provides a protective microenvironment on conjugation. This increases the active life of the fusion molecule in the circulation and prevents it from being recognized by the immune system. The PSA polymer is naturally found in the human body. It was adopted by certain bacteria which evolved over millions of years to coat their walls with it. These naturally polysialylated bacteria were then able, by virtue of molecular mimicry, to foil the body's defense system. PSA, nature's ultimate stealth technology, can be easily produced from such bacteria in large quantities and with predetermined physical characteristics. Bacterial PSA is completely non-immunogenic, even when coupled to proteins, as it is chemically identical to PSA in the human body.

### Targeting Moieties

The disclosure provides a fusion molecule comprising a SMAGP extracellular domain (ECD) and a targeting moiety that binds to a target molecule on a cell. Those of ordinary skill in the art will be aware of suitable targeting moieties, for example, a protein, a peptide, an antibody, a nucleic acid, a nucleic acid analog, a carbohydrate, and a small molecule. Other non-limiting examples of targeting moieties include sugars and polymers. The targeting moiety is selected based on the target cell type, tissue, or organ to allow sufficiently specific delivery of the fusion molecule to the desired target.

In some embodiments, the targeting moiety binds to a target molecule on a cell selected from the group consisting of a diseased cell, a senescent cell, a cancer cell, a B cell, a T cell, and a dendritic cell.

In some embodiments, the targeting moiety binds to a target molecule on a diseased cell or a senescent cell, and the target molecule is selected from the group consisting of DEP1, NTAL, EBP50, STX4, VAMP3, ARMCX3, B2MG, LANCL1, PLD3, VPS26A, DPP4, SCAMP4, MICA/B, TNFRSF10D/CD264, NOTCH1, NOTCH3, CD36, oxidized Vimentin, ICAM-1, uPAR, DEP1/PTPRJ/CD148, CD264, TNFRSF10D, TRAILR4, and CD26.

In some embodiments, the targeting moiety binds to a target molecule on a cancer cell and the target molecule is selected from the group consisting of ADAM9, B7-H3/CD276, BCMA, CA6, CA9, CD19, CD20, CD22, CD30, CD33, CD37, CD38, CD70, CD79b, CD123, CD138, CD157/BST1, P-cadherin CDH3, CEACAM5, CEACAM6, CLDN6, CLDN18.2, DLL3, EGFR, EGFRvIII, ENPP3, ENTPD2, EpCAM, FGR3, FLT3, FOLR1, GPA33, GPC3, GPNMB, GPRC5D, GUCY2C, Her2, HHLA2, LAMP1, SLC39A6/Liv-1, Mesothelin, MUC16/CA125, MUC17, SLC34A1/NaPi2a, Nectin 4, CD274/PD-L1, PSCA, PSMA/FOLH1, PVR, PVRIg, ROR1, SLITRK6, SSTR2, STEAP1, TROP2, and TMEM97.

In some embodiments, the targeting moiety binds to a target molecule on a cancer cell and the target molecule is selected from the group consisting of ADAM9, B7-H3/CD276, CEACAM6, CLDN6, DLL3, ENTPD2, FOLR1, GPA33, GPC3, Mesothelin, Nectin 4, PSMA/FOLH1, PVR, and SLITRK6.

In some embodiments, the targeting moiety binds to a target molecule on a cancer cell from a liquid tumor, and the target molecule is selected from the group consisting of BCMA, CD19, CD20, CD22, CD30, CD33, CD37, CD38, CD70, CD79b, CD123, CD138, CD157/BST1, FGR3, and FLT3.

In some embodiments, the targeting moiety binds to a target molecule on a cancer cell from a solid tumor, and the target molecule is selected from the group consisting of ADAM9, B7-H3/CD276, CA6, CA9, P-cadherin CDH3, CEACAM5, CEACAM6, CLDN6, CLDN18.2, DLL3, EGFR, EGFRvIII, ENPP3, ENTPD2, EpCAM, FOLR1, GPA33, GPC3, GPNMB, GPRC5D, GUCY2C, Her2, HHLA2, LAMP1, SLC39A6/Liv-1, Mesothelin, MUC16/CA125, MUC17, SLC34A1/NaPi2a, Nectin 4, CD274/PD-L1, PSCA, PSMA/FOLH1, PVR, PVRIg, ROR1, SLITRK6, SSTR2, STEAP1, TROP2, and TMEM97.

In some embodiments, the targeting moiety binds to a target molecule on a B cell, such as a B cell surface antigen. In some embodiments, the B cell surface antigen in CD20. In some embodiments, the targeting moiety that binds to a target molecule is an antibody or antigen-binding fragment thereof. In some embodiments, the targeting moiety is rituximab or an antigen-binding fragment thereof. Rituximab-containing fusion molecules are particularly useful in treating lymphoproliferative disorders, such as lymphoma, or rheumatoid arthritis (RA).

### Payload Molecules

The disclosure provides a fusion molecule comprising a SMAGP extracellular domain (ECD), e.g., a glycosylated or aglycosylated SMAGP ECD, and a payload molecule.

In some embodiments, the payload molecule is selected from the group consisting of an mRNA, an miRNA, a cRNA, a tRNA, an siRNA, an sgRNA, an antisense oligonucleotide, a peptide, a virus, a viral RNA genome, a viral DNA genome, a vector, a plasmid, a DNA, a radionuclide, and a drug, e.g., a small molecule drug. In some embodiments, the payload molecule is enclosed in or attached to a nanoparticle. In some embodiments, the payload molecule is a virus selected from an adeno-associated virus or a lentivirus. In some embodiments, the payload molecule is a DNA vector. In some embodiments, the drug is a cytotoxic drug.

In some embodiments, the payload molecule modulates the activity of a cell expressing CLEC10A (also known as C-type lectin domain family 10 member A, MGL, MGL1, or MMGL; see, e.g., UniProtKB Reference Sequence Q8IUN9 (e.g., Q8IUN9-1, as updated 2003 Mar. 1), and Q8IUN9-2) which sets forth an exemplary human CLEC10A) or CD177 (also known as Cluster of Differentiation 177; see, e.g., UniProtKB Reference Sequence Q8N6Q3 (e.g., Q8N6Q3-1, as updated 2006 Apr. 4), which sets forth an exemplary human CD177). In some embodiments, the payload molecule is designed or selected to modulate (e.g., enhance or inhibit) the activity of a neutrophil, e.g., a CD177-expressing neutrophil, as described, e.g., in Völs et al. (2022) Front Immunol. 13:1003871 (which is hereby incorporated by reference in its entirety), e.g., in order to treat a disease or disorder in which neutrophils play a role, e.g., atherosclerosis, a cancer (as described, e.g., in Astarita et al. (2021) PLOS One 16 (12): e0260800; and Kim et al. (2021) Nat Commun. 12 (1): 5764 (each of which is hereby incorporated by reference in its entirety)), an inflammatory disease or disorder (as described, e.g., in Filep (2022) Front Immunol. 13:866747 (which is hereby incorporated by reference in its entirety)), or an infectious disease. For example, the payload molecule can be selected from the group consisting of a small molecule drug that inhibits the NADPH oxidase complex responsible for the production of ROS (e.g., diphenyleneiodonium or stelazine), a degranulation inhibitor (e.g., nexinhib-20), and an enhancer of neutrophil apoptosis (e.g., roscovitine). In some embodiments, the payload molecule is a nucleic acid molecule, e.g., an mRNA, an miRNA, a cRNA, a tRNA, an siRNA, an sgRNA, an antisense oligonucleotide, a viral RNA genome, a viral DNA genome, a vector, a plasmid, or a DNA, e.g., for delivery to a neutrophil, e.g., a CD177-expressing neutrophil, or another CD177-expressing cell, e.g., for generation of engineered chimeric antigen receptor neutrophils (CAR-N), as described, e.g., in Chang et al. (2022) Cell Rep. 40 (3): 111128 (which is hereby incorporated by reference in its entirety). In some embodiments, the payload molecule is conjugated, e.g., directly to the SMAGP ECD, or the payload molecule is enclosed in or attached to a nanoparticle, e.g., a lipid nanoparticle. In some embodiments, the payload molecule is attached to a mutant or variant SMAGP ECD. In some embodiments, the payload molecule is attached to an aglycosylated SMAGP ECD.

In some embodiments, the nanoparticle is a lipid nanoparticle and the linker is linked to a lipid component of the lipid nanoparticle. In some embodiments, the lipid component of the lipid nanoparticle to which the linker is linked is selected from the group consisting of an ionizable lipid, a pegylated lipid, a helper lipid, and cholesterol. In some embodiments, the helper lipid is selected from the group consisting of 1,2-Distearoyl-sn-glycero-3-phosphorylethanolamine (DSPE); 1,2-distearoyl-sn-glycero-3-phosphocholine (DSPC); 1,2-dioleoyl-sn-glycero-3-phosphoethanolamine (DOPE); and 1,2-dioleoyl-sn-glycero-3-phosphoethanolamine-N-dibenzocyclooctyl (DBCO PE). In some embodiments, the helper lipid is DBCO PE, wherein the SMAGP ECD comprises an N-terminal azidohomoalanine, and wherein the linker comprises a clickable DARPin that binds to the DBCO PE and the N-terminal azidohomoalanine. In some embodiments, the pegylated lipid is selected from the group consisting of 1,2-distearoyl-sn-glycero-3-phosphoethanolamine-N-[maleimide(polyethylene glycol)-2000] (DSPE-PEG (2000) Maleimide); 1,2-distearoyl-sn-glycero-3-phosphoethanolamine-N-[maleimide(polyethylene glycol)-5000] (DSPE-PEG (5000) Maleimide); 1,2-dimyristoyl-rac-glycero-3-methoxypolyethylene glycol-2000 (DMG-PEG (2000)); and 2-[(polyethylene glycol)-2000]-N,N-ditetradecylacetamide (ALC-0159). In some embodiments, the pegylated lipid is DSPE-PEG (2000) Maleimide or DSPE-PEG (5000) Maleimide, and wherein the linker comprises a cysteine fused to the C terminus of the SMAGP ECD that binds to the pegylated lipid.

### Linkers

The disclosure provides fusion molecules comprising a SMAGP ECD linked to one or more moieties selected from the group consisting of a half-life extending moiety, a targeting moiety, and a payload molecule. The half-life extending moiety, targeting moiety, or payload molecule may be linked to the N-terminus or the C-terminus of a SMAGP ECD. Alternatively, the half-life extending moiety, targeting moiety, or payload molecule may to linked at a position other than the N-terminus or the C-terminus of a SMAGP ECD.

In some embodiments, the half-life extending moiety, targeting moiety, or payload molecule may be non-covalently linked to the SMAGP ECD. In some embodiments, the half-life extending moiety, targeting moiety, or payload molecule may be covalently linked to the SMAGP ECD.

In some embodiments, the half-life extending moiety, targeting moiety, or payload molecule may be linked (e.g., fused) directly to the N-terminus, C-terminus, or other region of a SMAGP ECD. In some embodiments, the half-life extending moiety, targeting moiety, or payload molecule is linked to the N-terminus, C-terminus, or other region of a SMAGP ECD via a linker. In some embodiments, the linker is a non-cleavable linker. As used herein, the term “non-cleavable linker” refers to a linker that is not readily cleaved by one or more of a given enzyme, chemical agent, or photo-irradiation. In some embodiments, the enzyme is a protease.

In some embodiments, the linker is a synthetic compound linker such as, for example, a chemical cross-linking agent. Non-limiting examples of suitable cross-linking agents that are available on the market include N-hydroxysuccinimide (NHS), disuccinimidylsuberate (DSS), bis(sulfosuccinimidyl) suberate (BS3), dithiobis(succinimidylpropionate) (DSP), dithiobis(sulfosuccinimidylpropionate) (DTSSP), ethyleneglycol bis(succinimidylsuccinate) (EGS), ethyleneglycol bis(sulfosuccinimidylsuccinate) (sulfo-EGS), disuccinimidyl tartrate (DST), disulfosuccinimidyl tartrate (sulfo-DST), bis[2-(succinimidooxycarbonyloxy)ethyl]sulfone (BSOCOES), and bis[2-(sulfosuccinimidooxycarbonyloxy)ethyl]sulfone (sulfo-BSOCOES).

As described above, Fc regions disclosed herein can comprise a hinge region or portion thereof. As such, the half-life extending moiety, targeting moiety, or payload molecule may be linked to the N-terminus or C-terminus of a SMAGP ECD via this hinge region. In some embodiments, one or more amino acids are included between the SMAGP ECD and the half-life extending moiety, targeting moiety, or payload molecule. In some embodiments, the one or more amino acids included between the SMAGP ECD and the half-life extending moiety, targeting moiety, or payload molecule are amino acids of a natural hinge region. In some embodiments, the SMAGP ECD is fused to the half-life extending moiety, targeting moiety, or payload molecule via a hinge region or a portion thereof. In some embodiments, the hinge region is an IgG hinge region, such as a human IgG hinge region. In some embodiments, the peptide linker is a portion of a hinge region comprising the amino acid sequence of EPKSX (SEQ ID NO: 4), wherein X is C or S.

In some embodiments, the linker is a peptide linker. Examples of peptide linkers are well-known and those of skill in the art could select a suitable peptide linker for use in linking a SMAGP ECD to a half-life extending moiety, targeting moiety, or payload molecule.

Peptide linkers may be of any length. In some embodiments, the length and amino acid composition of the linker peptide sequence can be optimized to vary the orientation and/or proximity of the moieties to one another to achieve a desired activity. In some embodiments, the peptide linker is between about 1 and about 100 amino acids in length, between about 8 and about 40 amino acids in length, or between about 15 amino acids and about 25 amino acids in length. In some embodiments, the peptide linker is between 1 and 100 amino acids in length, between 8 and 40 amino acids in length, or between 15 and 25 amino acids in length. In some embodiments, the peptide linker is about 8 amino acid in length, about 9 amino acids in length, about 10 amino acids in length, about 11 amino acids in length, about 12 amino acids in length, about 13 amino acids in length, about 14 amino acids in length, about 15 amino acids in length, about 16 amino acids in length, about 17 amino acids in length, about 18 amino acids in length, about 19 amino acids in length, about 20 amino acids in length, about 21 amino acids in length, about 22 amino acids in length, about 23 amino acids in length, about 24 amino acids in length, about 25 amino acids in length, about 26 amino acids in length, about 27 amino acids in length, about 28 amino acids in length, about 29 amino acids in length, about 30 amino acids in length, about 31 amino acids in length, about 32 amino acids in length, about 33 amino acids in length, about 34 amino acids in length, about 35 amino acids in length, about 36 amino acids in length, about 37 amino acids in length, about 38 amino acids in length, about 39 amino acids in length, or about 40 amino acids in length. In some embodiments, the peptide linker is 8 amino acid in length, 9 amino acids in length, 10 amino acids in length, 11 amino acids in length, 12 amino acids in length, 13 amino acids in length, 14 amino acids in length, 15 amino acids in length, 16 amino acids in length, 17 amino acids in length, 18 amino acids in length, 19 amino acids in length, 20 amino acids in length, 21 amino acids in length, 22 amino acids in length, 23 amino acids in length, 24 amino acids in length, 25 amino acids in length, 26 amino acids in length, 27 amino acids in length, 28 amino acids in length, 29 amino acids in length, 30 amino acids in length, 31 amino acids in length, 32 amino acids in length, 33 amino acids in length, 34 amino acids in length, 35 amino acids in length, 36 amino acids in length, 37 amino acids in length, 38 amino acids in length, 39 amino acids in length, or 40 amino acids in length.

In some embodiments, the peptide linker contains only glycine and/or serine residues (e.g., glycine-serine linker or GS linker). Examples of such peptide linkers include: Gly(x) Ser, where x is 0 to 6; or Ser Gly(x), where x is 0 to 6; (Gly Gly Gly Ser)n (SEQ ID NO: 35), wherein n is an integer of one or more; and (Ser Gly Gly Gly)n (SEQ ID NO: 36), wherein n is an integer of one or more. In some embodiments, the linker peptides are modified such that the amino acid sequence GSG (that occurs at the junction of traditional Gly/Ser linker peptide repeats) is not present. For example, in some embodiments, the peptide linker includes an amino acid sequence selected from the group consisting of: (GGGXX)nGGGGS (SEQ ID NO: 37) and GGGGS (XGGGS)n (SEQ ID NO: 38), where X is any amino acid that can be inserted into the sequence and not result in a polypeptide including the sequence GSG, and n is 0 to 4. In some embodiments, the sequence of a linker peptide is (GGGX₁X₂)nGGGGS (SEQ ID NO: 39), wherein X₁ is P and X₂ is S, and n is 0 to 4. In some other embodiments, the sequence of a linker peptide is (GGGX₁X₂)nGGGGS (SEQ ID NO: 40), wherein X₁ is G and X₂ is Q, and n is 0 to 4. In some other embodiments, the sequence of a linker peptide is (GGGX₁X₂)nGGGGS (SEQ ID NO: 41) and X₁ is G and X₂ is A and n is 0 to 4. In yet other embodiments, the sequence of a linker peptide is GGGGS (XGGGS)n (SEQ ID NO: 42), wherein X is P and n is 0 to 4. In some embodiments, a linker peptide of the disclosure comprises or consists of the amino acid sequence (GGGGA)₂GGGGS (SEQ ID NO: 43). In some embodiments, a linker peptide comprises or consists of the amino acid sequence (GGGGQ)₂GGGGS (SEQ ID NO: 44). In another embodiment, a linker peptide comprises or consists of the amino acid sequence (GGGPS)₂GGGGS (SEQ ID NO: 45). In another embodiment, a linker peptide comprises or consists of the amino acid sequence GGGGS(PGGGS)₂ (SEQ ID NO: 46). In yet a further embodiment, a linker peptide comprises or consists of the amino acid sequence GSGGS (SEQ ID NO: 47) or SGGSGS (SEQ ID NO: 48). In some embodiments, a linker peptide comprises or consists of the amino acid sequence GGGGGSGGGGSGGGSGGGGS (SEQ ID NO: 49). In some embodiments, the peptide linker comprises an amino acid sequence selected from the group consisting of SEQ ID NOs: 4, 35-49, 58-61, or a variant thereof comprising 1-5 amino acid changes.

In some embodiments, the peptide linker is a GS linker of about 20 amino acids in length. In some embodiments, the peptide linker is a GS linker of 20 amino acids in length.

### Polynucleotides, Vectors, and Methods of Production

The disclosure also provides polynucleotides that encode any of the fusion molecules or portions thereof described herein. In some embodiments, the polynucleotide encodes a SMAGP ECD of the disclosure. In some embodiments, the polynucleotide encodes an Fc region of the disclosure. In some embodiments, the polynucleotide encodes one or more of a half-life extending moiety, a targeting moiety, and a payload molecule. In some embodiments, the polynucleotide encodes a SMAGP ECD and an Fc region, and optionally a linker. In some embodiments, the polynucleotide encodes a SMAGP ECD and one or more of a half-life extending moiety, a targeting moiety, and a payload molecule, and optionally a linker. In some embodiments, the polynucleotides described herein are DNA molecules. In some embodiments, the polynucleotides described herein are RNA molecules.

In some embodiments, the polynucleotide comprises a nucleotide sequence that encodes a fusion molecule comprising an amino acid sequence at least 70%, 75%, 80%, 85%, 90%, 95%, 96%, 97%, 98%, or 99% identical to the amino acid sequence of any one of SEQ ID NOs: 19-34. In some embodiments, the polynucleotide comprises a nucleotide sequence that encodes a fusion molecule comprising the amino acid sequence of any one of SEQ ID NOs: 19-34. In some embodiments, the polynucleotide comprises a nucleotide sequence that encodes a fusion molecule consisting of the amino acid sequence of any one of SEQ ID NOs: 19-34.

In some embodiments, the polynucleotide comprises a first nucleotide sequence that encodes a SMAGP ECD and a second nucleotide sequence that encodes a half-life extending moiety, a targeting moiety, or a payload molecule. In some embodiments, the polynucleotide comprises a first nucleotide sequence that encodes a SMAGP ECD and an Fc region. In some embodiments, the first nucleotide sequence encodes a SMAGP ECD consisting of an amino acid sequence at least 70%, 75%, 80%, 85%, 90%, 95%, 96%, 97%, 98%, or 99% identical to the amino acid sequence of any one of SEQ ID NOs: 1-3. In some embodiments, the second nucleotide sequence encodes an Fc domain consisting of an amino acid sequence at least 70%, 75%, 80%, 85%, 90%, 95%, 96%, 97%, 98%, or 99% identical to the amino acid sequence of any one of SEQ ID NOs: 5-18 and 50-57. In some embodiments, the first nucleotide sequence encodes a SMAGP ECD comprising or consisting of the amino acid sequence of any one of SEQ ID NOs: 1-3. In some embodiments, the second nucleotide sequence encodes an Fc domain comprising or consisting of the amino acid sequence of any one of SEQ ID NOs: 5-18 and 50-57. In some embodiments, the first nucleotide sequence encodes a SMAGP ECD comprising or consisting of the amino acid sequence of any one of SEQ ID NOs: 1-3, and the second nucleotide sequence encodes an Fc domain comprising or consisting of the amino acid sequence of any one of SEQ ID NOs: 5-18 and 50-57.

In some embodiments, the first or second nucleotide sequence also encodes a linker. In some embodiments, the first nucleotide sequence encodes a SMAGP ECD and a linker and the second nucleotide sequence encodes an Fc region. In some embodiments, the first nucleotide sequence encodes a SMAGP ECD and the second nucleotide sequence encodes an Fc region and a linker. The linker can be any linker described herein, including a portion of a hinge region, a peptide linker, or a linker comprising the amino acid sequence of any one of SEQ ID NOs: 4, 35-49, or 58-61. In some embodiments, the linker consists of the amino acid sequence of any one of SEQ ID NOs: 4, 35-49, or 58-61.

Also provided herein are polynucleotides encoding a fusion molecule or portion thereof as provided above that are optimized, e.g., by codon/RNA optimization, replacement with heterologous signal sequences, and elimination of mRNA instability elements. Methods to generate optimized nucleic acids for recombinant expression by introducing codon changes and/or eliminating inhibitory regions in the mRNA can be carried out by adapting the optimization methods described in, e.g., U.S. Pat. Nos. 5,965,726; 6,174,666; 6,291,664; 6,414,132; and 6,794,498, accordingly, all of which are herein incorporated by reference in their entireties. For example, potential splice sites and instability elements (e.g., A/T or A/U rich elements) within the RNA can be mutated without altering the amino acids encoded by the nucleic acid sequences to increase stability of the RNA for recombinant expression. The alterations utilize the degeneracy of the genetic code, e.g., using an alternative codon for an identical amino acid. In an embodiment, it can be desirable to alter one or more codons to encode a conservative mutation, e.g., a similar amino acid with similar chemical structure and properties and/or function as the original amino acid.

The polynucleotides can be obtained, and the nucleotide sequence of the polynucleotides determined, by any method known in the art. Nucleotide sequences encoding fusion molecules or portions thereof described herein, and modified versions of these fusion molecules can be determined using methods well known in the art, i.e., nucleotide codons known to encode particular amino acids are assembled in such a way to generate a nucleic acid that encodes the fusion molecule or portion thereof. Such a polynucleotide encoding the protein can be assembled from chemically synthesized oligonucleotides (e.g., as described in Kutmeier G et al., (1994), BioTechniques 17:242-6, herein incorporated by reference in its entirety), which, briefly, involves the synthesis of overlapping oligonucleotides containing portions of the sequence encoding the fusion molecule or portion thereof, annealing, and ligating of those oligonucleotides, and then amplification of the ligated oligonucleotides by PCR.

Alternatively, a polynucleotide encoding a fusion molecule or portion thereof described herein can be generated from nucleic acid from a suitable source using methods well known in the art (e.g., PCR and other molecular cloning methods). For example, PCR amplification using synthetic primers hybridizable to the 3′ and 5′ ends of a known sequence can be performed using genomic DNA obtained from hybridoma cells producing the polypeptide of interest. Such PCR amplification methods can be used to obtain nucleic acids comprising the sequence encoding the fusion molecule or portion thereof. The amplified nucleic acids can be cloned into vectors for expression in host cells and for further cloning.

If a clone containing a nucleic acid encoding a particular polypeptide is not available, but the sequence of the polypeptide is known, a nucleic acid encoding the polypeptide can be chemically synthesized or obtained from a suitable source (e.g., a cDNA library generated from, or nucleic acid, preferably poly A+ RNA, isolated from any tissue or cells expressing the polypeptide described herein) by PCR amplification using synthetic primers hybridizable to the 3′ and 5′ ends of the sequence or by cloning using an oligonucleotide probe specific for the particular gene sequence to identify, e.g., a cDNA clone from a cDNA library that encodes the polypeptide. Amplified nucleic acids generated by PCR can then be cloned into replicable cloning vectors using any method well known in the art.

DNA encoding fusion molecules or portions thereof described herein can be readily isolated and sequenced using conventional procedures. Once isolated, the DNA can be placed into expression vectors, which are then transfected into host cells such as E. coli cells, simian COS cells, Chinese hamster ovary (CHO) cells (e.g., CHO cells from the CHO GS System™ (Lonza)), or myeloma cells that do not otherwise produce the molecules described herein.

Also provided are polynucleotides that hybridize under high stringency, intermediate or lower stringency hybridization conditions to polynucleotides that encode a fusion molecule or portion thereof described herein.

Hybridization conditions have been described in the art and are known to one of skill in the art. For example, hybridization under stringent conditions can involve hybridization to filter-bound DNA in 6× sodium chloride/sodium citrate (SSC) at about 45° C. followed by one or more washes in 0.2×SSC/0.1% SDS at about 50-65° C.; hybridization under highly stringent conditions can involve hybridization to filter-bound nucleic acid in 6×SSC at about 45° C. followed by one or more washes in 0.1×SSC/0.2% SDS at about 68° C. Hybridization under other stringent hybridization conditions is known to those of skill in the art and has been described, see, e.g., Ausubel F M et al., eds., (1989) Current Protocols in Molecular Biology, Vol. I, Green Publishing Associates, Inc. and John Wiley & Sons, Inc., New York at pages 6.3.1-6.3.6 and 2.10.3, which is herein incorporated by reference in its entirety.

In an aspect, provided herein are cells (e.g., host cells) expressing (e.g., recombinantly) a fusion molecule or portion thereof described herein, and related polynucleotides and expression vectors. Provided herein are vectors (e.g., expression vectors) comprising polynucleotides comprising nucleotide sequences encoding a fusion molecule or portion thereof described herein for recombinant expression in host cells, preferably in mammalian cells (e.g., CHO cells). Also provided herein are host cells comprising such vectors for recombinantly expressing fusion molecules or portions thereof described herein. In an aspect, provided herein are methods for producing a fusion molecule or portion thereof described herein, comprising expressing the fusion molecule or portion thereof from a host cell.

Recombinant expression of a protein described herein generally involves construction of an expression vector containing a polynucleotide that encodes the polypeptide. Once a polynucleotide encoding a polypeptide described herein has been obtained, the vector for the production of the polypeptide can be produced by recombinant DNA technology using techniques well known in the art. Thus, methods for preparing a fusion molecule or portion thereof by expressing a polynucleotide containing a polypeptide encoding nucleotide sequence are described herein. Methods which are well known to those skilled in the art can be used to construct expression vectors containing polypeptide coding sequences and appropriate transcriptional and translational control signals. These methods include, for example, in vitro recombinant DNA techniques, synthetic techniques, and in vivo genetic recombination. Also provided are replicable vectors comprising a nucleotide sequence encoding containing a fusion molecule or portion thereof polypeptide described herein, operably linked to a promoter. Such vectors can, for example, include the nucleotide sequence encoding an Fc region of the polypeptide (see, e.g., International Publication Nos. WO 86/05807 and WO 89/01036; and U.S. Pat. No. 5,122,464, which are herein incorporated by reference in their entireties).

An expression vector can be transferred to a cell (e.g., host cell) by conventional techniques and the resulting cells can then be cultured by conventional techniques to produce a fusion molecule or portion thereof described herein. Thus, provided herein are host cells containing a polynucleotide encoding containing a fusion molecule described herein or portions thereof.

In some embodiments, the vector is a non-viral vector. Exemplary non-viral vectors include, but are not limited to, plasmid DNA, transposons, episomal plasmids, minicircles, ministrings, and oligonucleotides (e.g., mRNA, naked DNA). In some embodiments, the vector is a DNA plasmid vector.

In some embodiments, the vector is a viral vector. Viral vectors can be replication competent or replication incompetent. Viral vectors can be integrating or non-integrating. A number of viral based systems have been developed for gene transfer into mammalian cells, and a suitable viral vector can be selected by a person of ordinary skill in the art. Exemplary viral vectors include, but are not limited to, adenovirus vectors (e.g., adenovirus 5), adeno-associated virus (AAV) vectors (e.g., AAV2, 3, 5, 6, 8, 9), retrovirus vectors (MMSV, MSCV), lentivirus vectors (e.g., HIV-1, HIV-2), gammaretrovirus vectors, herpes virus vectors (e.g., HSV1, HSV2), alphavirus vectors (e.g., SFV, SIN, VEE, M1), flavivirus (e.g., Kunjin, West Nile, Dengue virus), rhabdovirus vectors (e.g., rabies virus, VSV), measles virus vector (e.g., MV-Edm), Newcastle disease virus vectors, poxvirus vectors (e.g., VV), measles virus, and picornavirus vectors (e.g., Coxsackievirus).

In some embodiments, the vector or expression cassette comprises one or more additional elements. Additional elements include, but are not limited to, promoters, enhancers, polyadenylation (polyA) sequences, and selection genes.

In some embodiments, the vector comprises a polynucleotide sequence that encodes an amino acid sequence at least 90%, 95%, 96%, 97%, 98%, 99%, or 100% identical to an amino acid sequence recited in any of Tables 1-3. In some embodiments, the vector comprises a polynucleotide sequence that encodes an amino acid sequence recited in any of Tables 1-3.

### Pharmaceutical Compositions

The present disclosure provides compositions (e.g., pharmaceutical compositions) comprising a fusion molecule described herein, a nucleic acid molecule (e.g., an expression vector) encoding the fusion molecule, or a host cell expressing the fusion molecule. The pharmaceutical compositions described herein are formulated with suitable carriers, excipients, and other agents that provide improved transfer, delivery, tolerance, and the like. A multitude of appropriate formulations can be found in the formulary known to all pharmaceutical chemists: Remington's Pharmaceutical Sciences, Mack Publishing Company, Easton, PA. These formulations include, for example, powders, pastes, ointments, jellies, waxes, oils, lipids, lipid (cationic or anionic) containing vesicles (such as LIPOFECTIN™, Life Technologies, Carlsbad, CA), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also, Powell et al., “Compendium of excipients for parenteral formulations” PDA (1998) J Pharm Sci Technol 52:238-311.

The dose of a fusion molecule described herein, a nucleic acid molecule (e.g., an expression vector) encoding the fusion molecule, or a host cell expressing the fusion molecule administered to a patient may vary depending upon the age and the size of the patient, target disease, conditions, route of administration, and the like. The preferred dose is typically calculated according to body weight or body surface area. Depending on the severity of the condition, the frequency and the duration of the treatment can be adjusted. Effective dosages and schedules for administering the fusion proteins or component peptides described herein or the nucleic acid molecules, or the expression vectors that encode them may be determined empirically; for example, patient progress can be monitored by periodic assessment, and the dose adjusted accordingly. Moreover, interspecies scaling of dosages can be performed using well-known methods in the art (e.g., Mordenti et al., 1991, Pharmaceut. Res. 8:1351).

Various delivery systems are known and can be used to administer the pharmaceutical composition disclosed herein, e.g., encapsulation in liposomes, microparticles, microcapsules, recombinant cells capable of expressing the mutant viruses, receptor mediated endocytosis (see, e.g., Wu et al., 1987, J. Biol. Chem. 262:4429-4432). Methods of introduction include, but are not limited to, intradermal, intramuscular, intraperitoneal, intravenous, subcutaneous, intranasal, epidural, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

Any pharmaceutical composition described herein can be delivered subcutaneously or intravenously with a standard needle and syringe. In addition, with respect to subcutaneous delivery, a pen delivery device readily has applications in delivering a pharmaceutical composition disclosed herein. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that Contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

In certain situations, the pharmaceutical composition can be delivered in a controlled release system. In one embodiment, a pump may be used (see, Langer, supra; Sefton, 1987, CRC Crit. Ref. Biomed. Eng. 14:201). In another embodiment, polymeric materials can be used; see, Medical Applications of Controlled Release, Langer and Wise (eds.), 1974, CRC Pres., Boca Raton, Florida. In yet another embodiment, a controlled release system can be placed in proximity of the composition's target, thus requiring only a fraction of the systemic dose (see, e.g., Goodson, 1984, in Medical Applications of Controlled Release, supra, vol. 2, pp. 115-138). Other controlled release systems are discussed in the review by Langer, 1990, Science 249:1527-1533.

The injectable preparations may include dosage forms for intravenous, subcutaneous, intracutaneous, and intramuscular injections, drip infusions, etc. These injectable preparations may be prepared by methods publicly known. For example, the injectable preparations may be prepared, e.g., by dissolving, suspending, or emulsifying any of the fusion molecules described herein in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared is preferably filled in an appropriate ampoule.

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc.

### Methods of Treatment

The present disclosure provides methods comprising administering to a subject in need thereof a composition (e.g., a pharmaceutical composition, as described herein) comprising a fusion molecule described herein, a nucleic acid molecule (e.g., an expression vector) encoding the fusion molecule, or a host cell expressing the fusion molecule. In certain embodiments, the present disclosure provides a fusion molecule described herein, a polynucleotide encoding the fusion molecule, an expression vector comprising a polynucleotide encoding the fusion molecule, a nanoparticle comprising the fusion molecule, or a composition (e.g., a pharmaceutical composition, as described herein) comprising the fusion molecule, for use in medicine.

In some embodiments, the present disclosure provides methods for modifying an activity of a leukocyte, comprising contacting the leukocyte with an effective amount of a fusion molecule described herein, a nucleic acid molecule (e.g., an expression vector) encoding the fusion molecule, or a host cell expressing the fusion molecule. In some embodiments, the leukocyte is selected from the group consisting of myeloid cells, macrophages (e.g., M0 macrophages, M1 macrophages, M2 macrophages, M2a macrophages, M2b macrophages, M2c macrophages, or M2d macrophages), Kupffer cells, histiocytes, microglia, osteoclasts, dendritic cells, mast cells, neutrophils, regulatory T cells, tumor-infiltrating regulatory T cells, and granulocytes. In some embodiments, the activity of the leukocyte comprises one or more of phagocytosis, cytokine production, chemokine production, antigen presentation, growth factor production, and protease production. In some embodiments, the activity comprises phagocytosis of a population of cells or cell-like structures. In some embodiments, the population of cells or cell-like structures is selected from the group consisting of cancer cells, immune cells, neurons, red blood cells, and platelets. In some embodiments, the activity of the leukocyte is increased. In some embodiments, the activity of the leukocyte is reduced. In some embodiments, the activity of the leukocyte is increased or reduced by at least about 5%, at least about 10%, at least about 15%, at least about 20%, at least about 25%, at least about 30%, at least about 35%, at least about 40%, at least about 50%, at least about 60%, at least about 70%, at least about 80%, at least about 90%, at least about 100%, at least about 125%, at least about 150%, at least about 175%, or at least about 200% of the activity.

The disclosure also provides methods for treating diseases or disorders in a subject in need thereof, comprising administering to the subject a therapeutically effective amount of a fusion molecule described herein, a nucleic acid molecule (e.g., an expression vector) encoding the fusion molecule, or a host cell expressing the fusion molecule. Non-limiting examples of diseases or disorders include, for example, an autoimmune disorder (e.g., an autoimmune disorder characterized by excessive phagocytic activity), a lymphoproliferative disorder, macrophage activation syndrome (MAS), a cytokine-related disorder, a central nervous system (CNS) disease, a disease of hyperactivated microglia, a disease of overactivated osteoclasts, osteoporosis, bone degradation and/or metastasis associated with cancer, multiple myeloma bone disease, systemic juvenile idiopathic arthritis, an allergy, a cancer, a cancer with low PD-L1 expression, a cancer that is refractory to PD-1 and/or PD-L1 inhibitors, a disease or disorder characterized by multinucleated giant cells, and an atopic disease (e.g., atopic dermatitis). As used herein, a “cancer with low PD-L1 expression” is a cancer with a tumor that express PD-L1 with Tumor Proportion Score (TPS) or Combined Positive Score (CPS) of ≥1%, ≥10%, or ≥50%, as determined by an FDA-approved test. See, e.g., https://www.keytrudahcp.com/biomarker-testing/pd-11/

In some embodiments, the autoimmune disorder is selected from the group consisting of allogenic islet graft rejection, alopecia areata, ankylosing spondylitis, antiphospholipid syndrome, autoimmune Addison's disease, Alzheimer's disease, antineutrophil cytoplasmic autoantibodies (ANCA), autoimmune diseases of the adrenal gland, autoimmune hemolytic anemia, autoimmune hepatitis, autoimmune myocarditis, autoimmune neutropenia, autoimmune oophoritis and orchitis, immune thrombocytopenia (ITP or idiopathic thrombocytopenia purpura, idiopathic thrombocytopenia purpura, immune mediated thrombocytopenia, or primary immune thrombocytopenia), autoimmune urticaria, Behçet's disease, bullous pemphigoid (BP), cardiomyopathy, Castleman's syndrome, celiac spruce-dermatitis, chronic fatigue immune disfunction syndrome, chronic inflammatory demyelinating polyneuropathy (CIDP), Churg-Strauss syndrome, cicatricial pemphigoid, CREST syndrome, cold agglutinin disease, Crohn's disease, dilated cardiomyopathy, discoid lupus, epidermolysis bullosa acquisita, essential mixed cryoglobulinemia, factor VIII deficiency, fibromyalgia-fibromyositis, glomerulonephritis, Grave's disease, Guillain-Barre, Goodpasture's syndrome, graft-versus-host disease (GVHD), Hashimoto's thyroiditis, hemophagocytic lymphohistiocytosis (HLH), hemophilia A, idiopathic inflammatory myopathics (IIMs), idiopathic membranous neuropathy, idiopathic pulmonary fibrosis, IgA neuropathy, IgM polyneuropathies, immune thrombocytopenia (ITP), immune-mediated necrotizing myopathy (IMNM), juvenile arthritis, Kawasaki's disease, lichen planus, lichen sclerosus, lupus erythematosus, lupus nephritis, Ménière's disease, mixed connective tissue disease, mucous membrane pemphigoid, multiple sclerosis, Type 1 diabetes mellitus, multifocal motor neuropathy (MMN), myasthenia gravis (MG), generalized myasthenia gravis (gMG), myositis, parancoplastic bullous pemphigoid, pemphigoid gestationis, pemphigus vulgaris (PV), pemphigus foliaccus (PF), pernicious anemia, polyarteritis nodosa, polychrondritis, polyglandular syndromes, polymyalgia rheumatica, polymyositis, dermatomyositis (DM), necrotizing autoimmune myopathy (NAM), AntiSynthetase Syndrome (AsyS), primary agammaglobulinemia, primary biliary cirrhosis, psoriasis, psoriatic arthritis, relapsing polychondritis, Raynaud's phenomenon, Reiter's syndrome, rheumatoid arthritis, sarcoidosis, scleroderma, Sjögren's syndrome, solid organ transplant rejection, stiff-man syndrome, systemic lupus erythematosus, Takayasu's arteritis, toxic epidermal necrolysis (TEN), Stevens-Johnson syndrome (SJS), temporal arteritis/giant cell arteritis, thrombotic thrombocytopenia purpura, ulcerative colitis, uveitis, dermatitis herpetiformis vasculitis, anti-neutrophil cytoplasmic antibody-associated vasculitis, vitiligo, and Wegner's granulomatosis.

In some embodiments, the lymphoproliferative disorder is selected from the group consisting of chronic lymphocytic leukemia, acute lymphocytic leukemia, hairy cell leukemia, large granular lymphocyte disorders, lymphocytosis, natural killer cell leukemia, prolymphocytic leukemia, follicular lymphoma, hemophagocytic lymphohistiocytosis (HLH), B-cell lymphomas, T-cell lymphomas, and multiple myeloma. Fusion molecules comprising a targeting moiety comprising rituximab or an antigen-binding fragment thereof are particularly suitable for treating a lymphoproliferative disorder.

In some embodiments, the CNS disease is Alzheimer's disease, schizophrenia, or Huntington's disease.

In some embodiments, the bone degradation and/or metastasis associated with cancer results from a disease or disorder selected from multiple myeloma, breast cancer, and prostate cancer.

Non-limiting examples of cancers than can be treated with the fusion molecules or compositions disclosed herein include a solid tumor, a hematologic cancer, leukemia, lymphoma, osteosarcoma, rhabdomyosarcoma, neuroblastoma, kidney cancer, renal transitional cell cancer, bladder cancer, Wilm's cancer, ovarian cancer, pancreatic cancer, breast cancer (e.g., characterized by a mutation in BRCA1 and/or BRCA2, or Her2+), prostate cancer, bone cancer, lung cancer (e.g., non-small cell lung cancer or small cell lung cancer), gastric cancer, colorectal cancer, cervical cancer, synovial sarcoma, head and neck cancer, squamous cell carcinoma, multiple myeloma, renal cell cancer, retinoblastoma, hepatoblastoma, hepatocellular carcinoma, melanoma, rhabdoid tumor of the kidney, Ewing's sarcoma, chondrosarcoma, brain cancer, glioblastoma, meningioma, pituitary adenoma, vestibular schwannoma, a primitive neuroectodermal tumor, medulloblastoma, astrocytoma, anaplastic astrocytoma, oligodendroglioma, ependymoma, choroid plexus papilloma, polycythemia vera, thrombocythemia, idiopathic myelofibrosis, soft tissue sarcoma, thyroid cancer, endometrial cancer, carcinoid cancer, liver cancer, epithelial cancer, and peritoneal cancer. In certain embodiments, the cancer is metastatic cancer, e.g., of the varieties described above.

In certain embodiments, the cancer is a solid tumor, a hematological cancer (e.g., leukemia, lymphoma, myeloma), and a metastatic lesion thereof. In one embodiment, the cancer is a solid tumor. Examples of solid tumors include malignancies, e.g., sarcomas and carcinomas (e.g., adenocarcinomas) of the various organ systems, such as those affecting lung, breast, lymphoid, gastrointestinal or colorectal, genitals and genitourinary tract (e.g., renal, urothelial, bladder cells), pharynx, CNS (e.g., brain, neural or glial cells), skin (e.g., melanoma), head and neck (e.g., head and neck squamous cell carcinoma (HNCC)), and pancreas. For example, melanoma, colon cancers, gastric cancer, rectal cancer, renal-cell carcinoma, breast cancer (e.g., a breast cancer that does not express one, two or all of estrogen receptor, progesterone receptor, or Her2/neu, e.g., a triple negative breast cancer; or Her2+ breast cancer), liver cancer, a lung cancer (e.g., a non-small cell lung cancer (NSCLC) e.g., a NSCLC with squamous and/or non-squamous histology) or small cell lung cancer), prostate cancer, cancer of head or neck (e.g., HPV+ squamous cell carcinoma), cancer of the small intestine and cancer of the esophagus.

In one embodiment, the cancer is a hematological cancer, for example, a leukemia, a lymphoma, or a myeloma. In one embodiment, the cancer is a leukemia, for example, acute lymphoblastic leukemia (ALL), acute myelogenous leukemia (AML), acute myeloblastic leukemia (AML), chronic lymphocytic leukemia (CLL), chronic myelogenous leukemia (CML), chronic myeloid leukemia (CML), chronic myelomonocytic leukemia (CMML), chronic lymphocytic leukemia (CLL), or hairy cell leukemia. In one embodiment, the cancer is a lymphoma, for example, B cell lymphoma, diffuse large B-cell lymphoma (DLBCL), activated B-cell like (ABC) diffuse large B cell lymphoma, germinal center B cell (GCB) diffuse large B cell lymphoma, mantle cell lymphoma, Hodgkin lymphoma, non-Hodgkin lymphoma, relapsed non-Hodgkin lymphoma, refractory non-Hodgkin lymphoma, recurrent follicular non-Hodgkin lymphoma, Burkitt lymphoma, small lymphocytic lymphoma, follicular lymphoma, lymphoplasmacytic lymphoma, or extranodal marginal zone lymphoma. In one embodiment, the cancer is a myeloma, for example, multiple myeloma.

In some embodiments, the disease or disorder is selected from the group consisting of H. pylori infection, COVID-19, severe COVID-19, IgA nephropathy, ovarian cancer, inflammatory bowel disease (IBD), acute-on-chronic liver failure (ACLF), antineutrophil cytoplasmic antibody-associated vasculitis (AAV), autoimmune vasculitis, asthma, acute respiratory distress syndrome (ARDS), chronic obstructive pulmonary disease (COPD), a disease in which regulatory T cells express CD177, Kawasaki disease, septic shock, renal cell carcinoma, hepatocellular carcinoma, breast cancer, lung cancer, and colorectal cancer.

Administration of the compositions according to the methods described herein may result in a reduction of the severity, signs, symptoms, or markers of a disease or disorder in a patient with the disease or disorder. By “reduction” in this context is meant a statistically significant decrease in such level. The reduction (absolute reduction or reduction of the difference between the elevated level in the subject and a normal level) can be, for example, at least about 20%, 25%, 30%, 35%, 40%, 45%, 50%, 55%, 60%, 65%, 70%, 75%, 80%, 85%, 90%, or 95%, or to below the level of detection of the assay used.

In an embodiment, the fusion molecule or composition thereof is administered to the subject simultaneously or sequentially with an additional therapeutic agent. In an embodiment, the additional therapeutic agent is an anti-inflammatory agent. In an embodiment, the additional therapeutic agent is a corticosteroid. In an embodiment, the additional therapeutic agent is rituximab, daclizumab, basiliximab, muronomab-cd3, infliximab, adalimumab, omalizumab, efalizumab, natalizumab, tocilizumab, eculizumab, golimumab, canakinumab, ustekinumab, or belimumab. In an embodiment, the additional therapeutic agent is a leucocyte depleting agent.

In an embodiment, the additional therapeutic agent is a B-cell depleting agent. In an embodiment, the B-cell depleting agent is an antibody. In an embodiment, the B-cell depleting antibody is an antibody that specifically binds to CD10, CD19, CD20, CD21, CD22, CD23, CD24, CD37, CD53, CD70, CD72, CD74, CD75, CD77, CD79a, CD79b, CD80, CD81, CD82, CD83, CD84, CD85, or CD86.

In an embodiment, the additional therapeutic agent is a chemotherapeutic agent. In an embodiment, the chemotherapeutic agent is paclitaxel.

In an embodiment, the additional therapeutic agent is an immune checkpoint modulating agent. In an embodiment, the immune checkpoint modulating agent is an anti-PD-1 agent or an anti-PD-L1 agent. In an embodiment, the immune checkpoint modulating agent is an anti-PD-1 antibody or an anti-PD-L1 antibody. In an embodiment, the additional therapeutic agent is a combination of a chemotherapeutic agent and an immune checkpoint modulating agent. In an embodiment, the chemotherapeutic agent is paclitaxel and the immune checkpoint modulating agent is an anti-PD-1 agent or an anti-PD-L1 agent. In an embodiment, the disease or disorder is ovarian cancer and the additional therapeutic agent is a combination of paclitaxel and an anti-PD-1 agent or an anti-PD-L1 agent.

The fusion molecules of the present disclosure can be further linked to or co-expressed with another functional molecule, e.g., another peptide or protein. For example, a fusion molecule can be functionally linked (e.g., by chemical coupling, genetic fusion, noncovalent association or otherwise) to one or more other molecular entities, such as an antibody or antibody fragment to produce a bispecific or a multispecific binding molecule with a second or additional binding specificity.

The Examples below demonstrate that CLEC10A and CD177 are receptors for SMAGP. Accordingly, a fusion molecule described herein can also be used to assay CLEC10A or CD177 (e.g., human CLEC10A or CD177) protein levels. In some embodiments, CLEC10A or CD177 (e.g., human CLEC10A or CD177) protein levels can be measured in a biological sample using classical immunohistological methods known to those of skill in the art, including immunoassays, such as the enzyme linked immunosorbent assay (ELISA), immunoprecipitation, or Western blotting. Suitable assay labels are known in the art and include enzyme labels, such as glucose oxidase; radioisotopes, such as iodine (1251, 1211), carbon (14C), sulfur (35S), tritium (3H), indium (121 In), and technetium (99Tc); luminescent labels, such as luminol; and fluorescent labels, such as fluorescein and rhodamine, and biotin. Such labels can be used to label a fusion molecule described herein. Alternatively, an antibody that recognizes a fusion molecule described herein can be labeled and used in combination with a fusion molecule to detect SMAGP (e.g., human SMAGP) protein levels. Therefore, in certain embodiments, the present disclosure relates to the use of a fusion molecule of the present disclosure for in vitro detection of any of SMAGP, CLEC10A, or CD177 (e.g., human SMAGP, CLEC10A, or CD177) protein in a biological sample. In a further embodiment, the present disclosure relates to the use of a fusion molecule of the disclosure, for assaying and/or detecting SMAGP, CLEC10A, or CD177 (e.g., human SMAGP, CLEC10A, or CD177) protein levels in a biological sample in vitro, optionally wherein the fusion molecule is conjugated to a radionuclide or detectable label, and/or carries a label described herein, and/or wherein an immunohistological method is used.

Assaying for the expression level of SMAGP, CLEC10A, or CD177 (e.g., human SMAGP, CLEC10A, or CD177) protein is intended to include qualitatively or quantitatively measuring or estimating the level of SMAGP, CLEC10A, or CD177 (e.g., human SMAGP, CLEC10A, or CD177) protein in a first biological sample either directly (e.g., by determining or estimating absolute protein level) or relatively (e.g., by comparing to the disease associated protein level in a second biological sample). SMAGP, CLEC10A, or CD177 (e.g., human SMAGP, CLEC10A, or CD177) polypeptide expression level in the first biological sample can be measured or estimated and compared to a standard or reference SMAGP, CLEC10A, or CD177 (e.g., human SMAGP, CLEC10A, or CD177) protein level, the standard being taken, for example, from a second biological sample obtained from an individual not having the disorder or being determined by averaging levels from a population of individuals not having the disorder. As will be appreciated in the art, once the “standard” SMAGP, CLEC10A, or CD177 (e.g., human SMAGP, CLEC10A, or CD177) polypeptide level is known, it can be used repeatedly as a standard for comparison. Therefore, in a further embodiment, the present disclosure relates to an in vitro method for assaying and/or detecting SMAGP, CLEC10A, or CD177 protein levels, for example human SMAGP, CLEC10A, or CD177 protein levels, in a biological sample, comprising qualitatively or quantitatively measuring or estimating the level of SMAGP, CLEC10A, or CD177 protein, for example of human SMAGP, CLEC10A, or CD177 protein, in a biological sample, by an immunohistological method. In certain embodiments, binding between SMAGP and CLEC10A or CD177 is measured using enzyme linked immunosorbent assay (ELISA), surface plasmon resonance (SPR), or flow cytometry.

As used herein, the term “biological sample” refers to any biological sample obtained from a subject, cell line, tissue, or other source of cells potentially expressing SMAGP, CLEC10A, or CD177 (e.g., human SMAGP, CLEC10A, or CD177). Methods for obtaining tissue biopsies and body fluids from animals (e.g., humans or cynomolgus monkeys) are well known in the art. Biological samples include peripheral blood mononuclear cells (PBMCs).

A fusion molecule described herein can be used for prognostic, diagnostic, monitoring, and screening applications, including in vitro and in vivo applications well known and standard to the skilled artisan and based on the present description. Prognostic, diagnostic, monitoring, and screening assays and kits for in vitro assessment and evaluation may be utilized to predict, diagnose, and monitor to evaluate patient samples or determining the suitability of a patient for a clinical trial of a drug or for the administration of a particular chemotherapeutic agent, a radiotherapeutic agent, or an antibody, including combinations thereof, versus a different agent or antibody. Therefore, in certain embodiments, the present disclosure relates to a fusion molecule and/or composition of the present disclosure for use as a diagnostic. In certain embodiments, the present disclosure relates to a fusion molecule and/or composition of the present disclosure for use in a method for the prediction, diagnosis and/or monitoring of a subject. In another embodiment, the present disclosure relates to the use of a fusion molecule of the disclosure, for predicting, diagnosing and/or monitoring of a subject by assaying and/or detecting human SMAGP, CLEC10A, or CD177 protein levels in a biological sample of the subject in vitro.

### Kits

Any of the compositions described herein may be comprised in a kit. In a non-limiting example, the kit comprises one or more of a fusion molecule described herein, a nucleic acid molecule (e.g., an expression vector) encoding the fusion molecule, or a host cell expressing the fusion molecule.

The kit may further include reagents or instructions for using a fusion molecule described herein, a nucleic acid molecule (e.g., an expression vector) encoding the fusion molecule, or a host cell expressing the fusion molecule, in a subject. It may also include one or more buffers.

The components of the kits may be packaged either in aqueous media or in lyophilized form. The container means of the kits will generally include at least one vial, test tube, flask, bottle, syringe, or other container means, into which a component may be placed, and preferably, suitably aliquoted. Where there is more than one component in the kit (labeling reagent and label may be packaged together), the kit also will generally contain a second, third, or other additional container into which the additional components may be separately placed. The kits may also comprise a second container means for containing a sterile, pharmaceutically acceptable buffer and/or other diluent. However, various combinations of components may be comprised in a vial. The kits of the present disclosure also typically include a means for containing a fusion molecule described herein, a nucleic acid molecule (e.g., an expression vector) encoding the fusion molecule, or a host cell expressing the fusion molecule, and any other reagent containers in close confinement for commercial sale.

When the components of the kit are provided in one and/or more liquid solutions, the liquid solution is an aqueous solution, with a sterile aqueous solution being particularly preferred. However, the components of the kit may be provided as dried powder(s). When reagents and/or components are provided as a dry powder, the powder can be reconstituted by the addition of a suitable solvent. It is envisioned that the solvent may also be provided in another container means.

### EXAMPLES

The examples of the present disclosure are offered by way of illustration and explanation, and are not intended to limit the scope of the present disclosure.

### Example 1. Generation and Characterization of SMAGP ECD-hIgG1 Fc Fusion Molecules

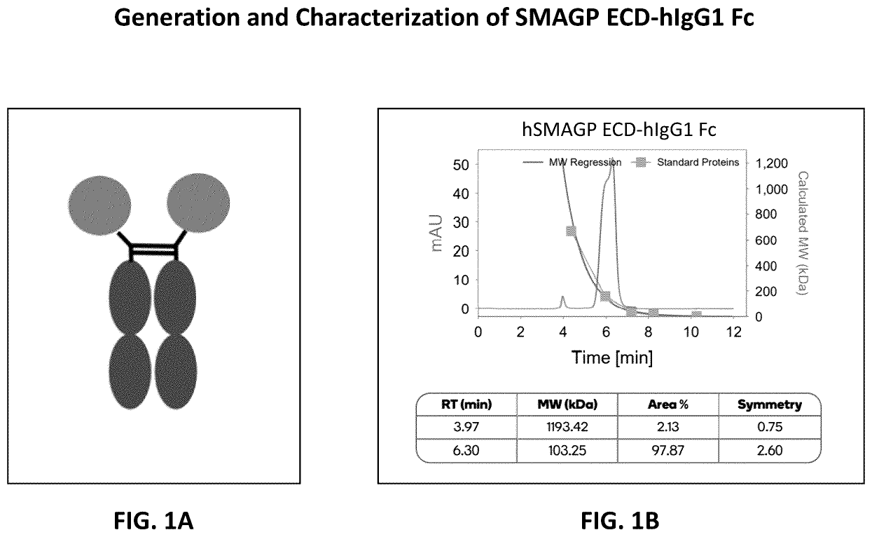

The extracellular domains of human SMAGP (Met1-Leu36) and cynomolgus monkey SMAGP (Met1-Leu35) were separately fused to a wild-type Fc domain of human IgG1, which includes the hinge, CH2, and CH3 domains. The Cys residue in the upper hinge region was mutated to Ser (C220S) in each molecule to avoid unpaired cysteine. The fusion molecules are referred to herein as hSMAGP ECD-hIgG1 Fc and cSMAGP ECD-hIgG1 Fc, respectively. A schematic of these fusion molecules is shown in FIG. 1A.

Protein expression vectors encoding each of these fusion molecules, together with an N-terminal leader sequence, were transfected into HEK293 cells. The fusion proteins were captured using protein A resin and buffer-exchanged into PBS, pH 7.0 buffer following elution. The resulting purified proteins were quantified by OD280. Purity of each protein was determined using a Perkin Elmer GXII capillary electrophoresis system, and the extent of protein aggregation was determined by HPLC-SEC. As shown in FIG. 1B, hSMAGP ECD-hIgG1 Fc was approximately 98% pure and exhibited minimal aggregation. The estimated MW (103.25 kDa) of hSMAGP ECD-hIgG1 Fc is significantly higher than the calculated MW (59.7 kDa), suggesting that the protein is glycosylated. Similar results were obtained for cSMAGP ECD-hIgG1 Fc.

### Example 2. Binding of SMAGP ECD-hIgG1 Fc to Human Macrophages, Neutrophils, and Monocytes

Human macrophages were generated from monocytes isolated from fresh peripheral blood leukapheresis products from two healthy human donors. Briefly, CD14⁺ monocytes were sorted via negative selection using Stem Cell Monocyte Enrichment kit. Monocytes were then cultured in RPMI with Glutamax+heat-inactivated fetal calf scrum and MCSF (40 ng/ml) for 6 days in 10 cm tissue-culture treated plates. Macrophages were detached using Accutase enzymatic digestion, washed and plated at 5e⁴/well in 96-well plates. Cells were then Fc-R blocked with 25 μg/ml antibodies to CD32, CD16 and CD64 in FACS buffer for 30 min at 4° C. Following washing, cells were then stained with hIgG1 Fc control, hSMAGP ECD-hIgG1 Fc, or cSMAGP ECD-hIgG1 Fc for 30 minutes in FACS buffer prior to secondary staining with anti-hIgG-647 for 30 minutes at 4° C. Following washing, macrophages were resuspended in FACS buffer plus NucBlue (2 drops/ml) and read on the flow cytometer.

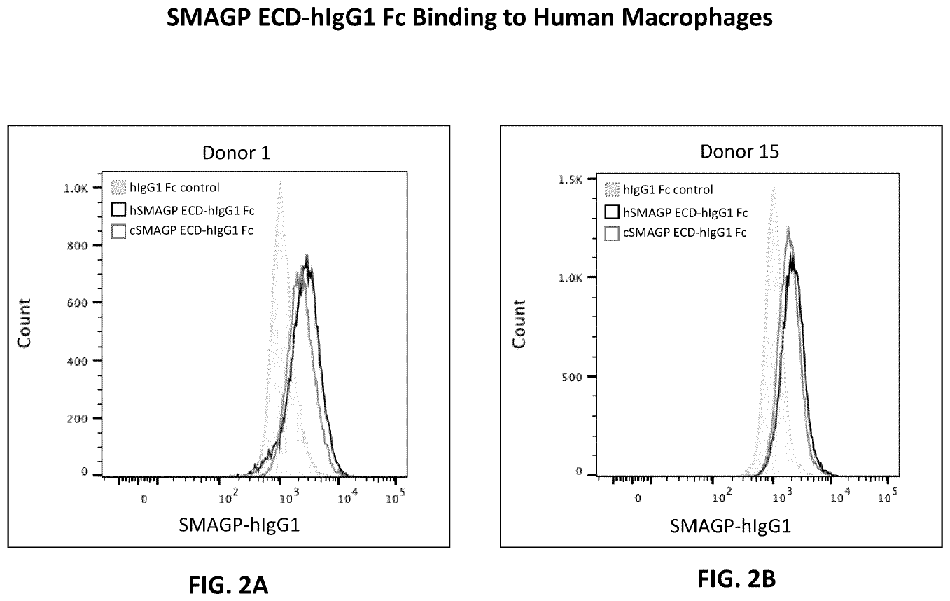

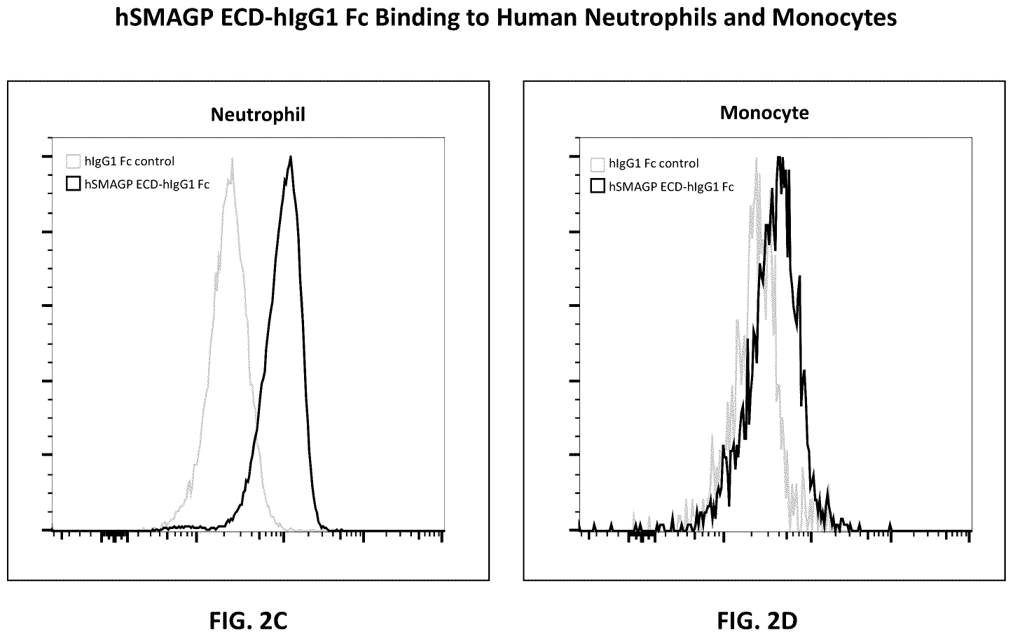

As shown in FIGS. 2A and 2B, binding of both hSMAGP ECD-hIgG1 Fc and cSMAGP ECD-hIgG1 Fc to primary human macrophages, relative to hIgG1 Fc control, was demonstrated. A repeat of this study yielded similar results.

In a further experiment, whole blood from healthy human donors was obtained. Red blood cells were lysed and then samples were Fc-R blocked with 25 μg/ml antibodies to CD32, CD16 and CD64 in FACS buffer for 30 min at 4° C. Following washing, cells were then stained with a viability dye, lineage markers (CD3, CD56, CD14, CD19 and CD15), and hIgG1 Fc control or hSMAGP ECD-hIgG1 Fc for 30 minutes in FACS buffer prior to secondary staining with anti-hIgG-647 for 30 minutes at 4° C. Following washing, cells were resuspended in FACS buffer and read on the flow cytometer.

As shown in FIG. 2C and FIG. 2D, binding of hSMAGP ECD-hIgG1 Fc, relative to hIgG1 Fc control, was demonstrated on both neutrophils (CD15+, FIG. 2C) and monocytes (CD14+, CD15−, FIG. 2D). A repeat of this study yielded similar results.

### Example 3. Inhibition of Human Macrophage Phagocytosis of Ramos Cells by hSMAGP ECD-hIgG1 Fc

Human macrophages were generated as described in Example 2. Following the monocyte culture step, macrophages were detached using Accutase enzymatic digestion, washed and plated at 3e5 cells/well in 96-well plates. Media was removed and cells washed prior to incubating with hSMAGP ECD-hIgG1 Fc (0.63, 1.25, 2.5, 5, 10, 20 or 40 μg/ml; 0.63 μg/ml condition only tested for one donor) or hIgG1 Fc control (20 or 40 μg/ml; 20 μg/ml condition only tested for one donor) for 30-45 minutes at 37° C. Ramos tumor cells were stained with 100 nM pHrodo-Red-SE in dPBS for 30 minutes, quenched and washed prior to addition to the culture at 605/well along with 10 μg/ml anti-CD47 antibody (clone B6H12). Images were acquired via Incucyte at 1 hour post-coculture and total red object area corresponding to tumor cell phagocytosis was evaluated. Additional controls included macrophages cocultured with Ramos SMAGP-overexpressing cells in the presence of anti-CD47 antibody and macrophages cocultured with Ramos parental cell lines in the absence of anti-CD47 antibody. The remaining tumor cells at the end of the assays were assessed by flow cytometry and were normalized to the 40 μg/ml IgG1 Fc control hIgG1-Fc group.

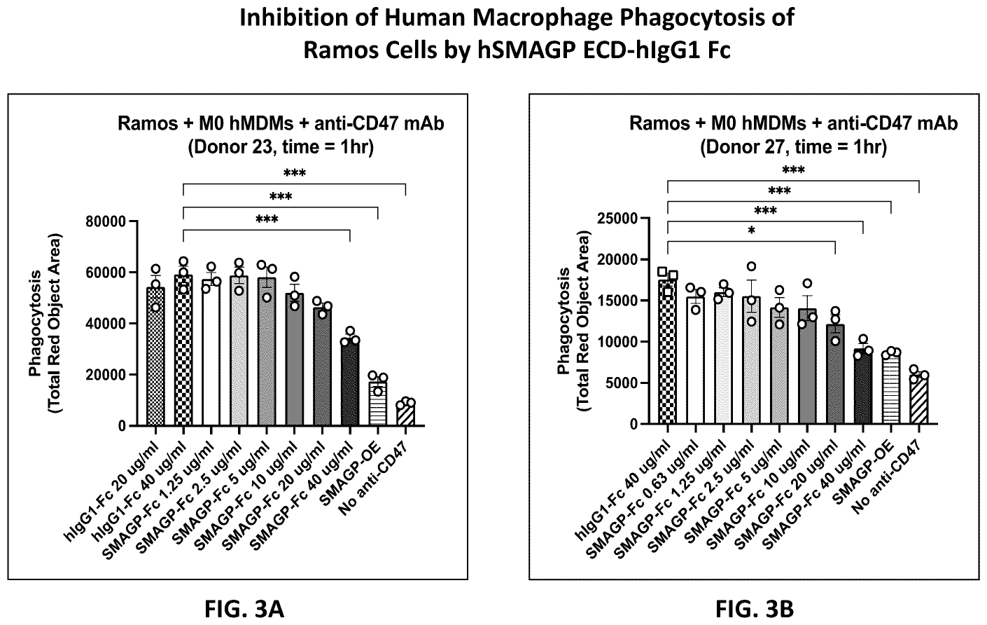

As shown in FIGS. 3A and 3B, hSMAGP ECD-hIgG1 Fc inhibited anti-CD47 blockade-induced phagocytosis of Ramos tumor cells by human macrophages from two separate donors, relative to IgG1 Fc control, in a dose-dependent fashion as measured at the 1-hour timepoint. As expected, Ramos SMAGP-over expressing tumor cells and Ramos cells without anti-CD47 were not well phagocytosed by human macrophages. For Donor 23-derived macrophages (FIG. 3A), statistical significance was observed for the 40 μg/ml hSMAGP ECD-hIgG1 Fc, the Ramos SMAGP-overexpressing cells, and the no-anti-CD47 conditions, each relative to the 40 g/ml IgG1 Fc control (using one-way ANOVA, p<0.001 (***)). For Donor 27-derived macrophages (FIG. 3B), statistical significance was observed for the 20 μg/ml hSMAGP ECD-hIgG1 Fc, the 40 μg/ml hSMAGP ECD-hIgG1 Fc, the Ramos SMAGP-overexpressing cells, and the no-anti-CD47 conditions, each relative to the 40 μg/ml IgG1 Fc control (using one-way ANOVA, p<0.033 (*), p<0.001 (***)).

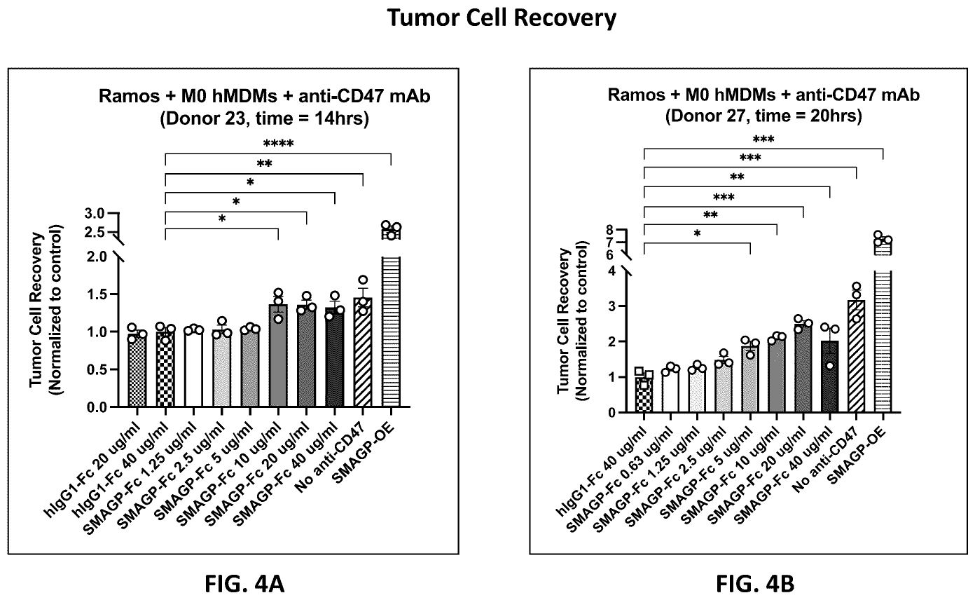

As shown in FIGS. 4A and 4B, incubation with hSMAGP ECD-hIgG1 Fc resulted in increased tumor cell recovery relative to incubation with IgG1 Fc control in a dose-dependent fashion, consistent with the inhibition of phagocytosis shown in FIGS. 3A and 3B. As expected, Ramos SMAGP-over expressing tumor cells and Ramos cells without anti-CD47 were not well phagocytosed by human macrophages, yielding increased tumor cell recoveries. For Donor 23-derived macrophages (FIG. 4A), statistical significance was observed for the 10, 20, and 40 μg/ml hSMAGP ECD-hIgG1 Fc, the Ramos SMAGP-overexpressing cells, and the no-anti-CD47 conditions, each relative to the 40 μg/ml IgG1 Fc control (using one-way ANOVA, p<0.033 (*), p<0.002 (**), p<0.0001 (****)). For Donor 27-derived macrophages (FIG. 4B), statistical significance was observed for the 5, 10, 20, and 40 μg/ml hSMAGP ECD-hIgG1 Fc, the Ramos SMAGP-overexpressing cells, and the no-anti-CD47 conditions, each relative to the 40 μg/ml IgG1 Fc control (using one-way ANOVA, p<0.033 (*), p<0.002 (**), p<0.001 (***)).

### Example 4. Inhibition of Human Macrophage Phagocytosis by hSMAGP ECD-hIgG1 Fc in A375 Tumor Cells

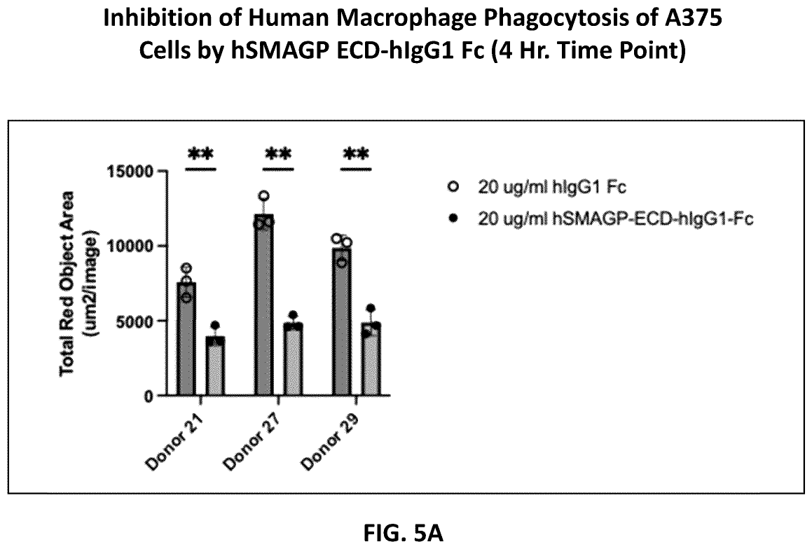

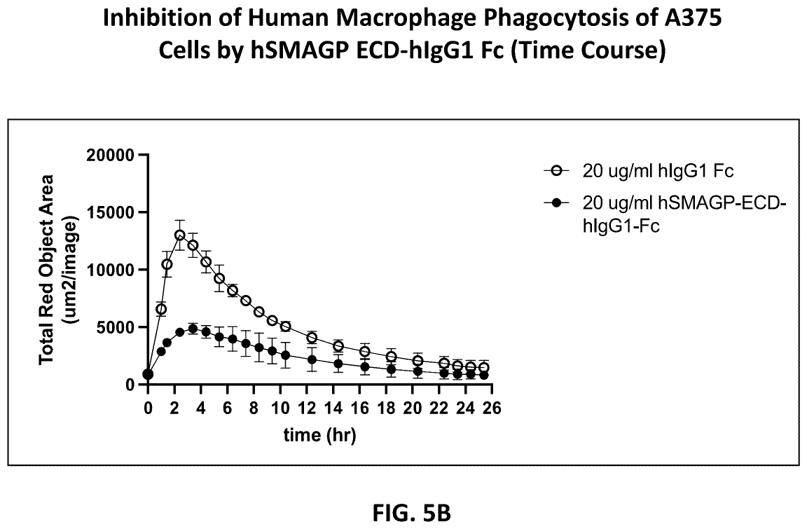

Human macrophages were generated from pan-monocytes isolated using negative selection or CD14⁺ selection, depending on the donor, from fresh peripheral blood leukapheresis products from healthy human donors. Monocytes were differentiated into macrophages by culturing for 7 days in the presence of recombinant human MCSF in RPMI media supplemented with 10% FBS. On Day 6, macrophages were detached and replated in MCSF containing media in 96 well plates and allowed to readhere overnight. On Day 7, A375 tumor cells were lifted, washed with PBS and stained with pH sensitive dye pHrodo-Red-SE. Macrophages were stimulated with hIgG1-Fc (Bio-X-Cell) or hSMAGP ECD-hIgG1 Fc for approximately 45 minutes. After this preincubation, 10 μg/ml anti-CD47 (magrolimab biosimilar, R&D systems) was added to all macrophage wells, and labeled tumor cells were added in an effector:target ratio of 1:1. Images were acquired via Incucyte for 25 hours post-coculture and total red object area corresponding to tumor cell phagocytosis was evaluated. Total red object area corresponding to tumor cell phagocytosis was quantified at 4 hrs. for three donors, showing a significant inhibition of phagocytosis for hSMAGP ECD-hIgG1 Fc relative to control for each donor (FIG. 5A). This observation was confirmed in a 25-hour phagocytosis time course for a representative donor (FIG. 5B). One-way ANOVA was used to assess statistical significance in FIG. 5A (p<0.05 (*), p<0.01 (**), p<0.001 (***), p<0.0001 (****)).

As A375 tumor cells do not express Fc receptors, the results of this experiment show that Fc receptor expression on the tumor cell is not required for inhibition of phagocytosis by hSMAGP ECD-hIgG1 Fc. It is speculated that hSMAGP ECD-hIgG1 Fc may bind macrophages and other myeloid cells through both the SMAGP ECD and the hIgG1 Fc domain, either in cis or in trans, while hSMAGP ECD-hIgG1 Fc may also bind tumor cells that express Fc receptors through the hIgG1 Fc domain, thus “studding” the tumor cell with hSMAGP ECD-hIgG1 Fc and mimicking an overexpressing tumor cell.

### Example 5. Inhibition of Human Macrophage Phagocytosis by hSMAGP ECD-hIgG1 Fc in the Presence of Rituximab

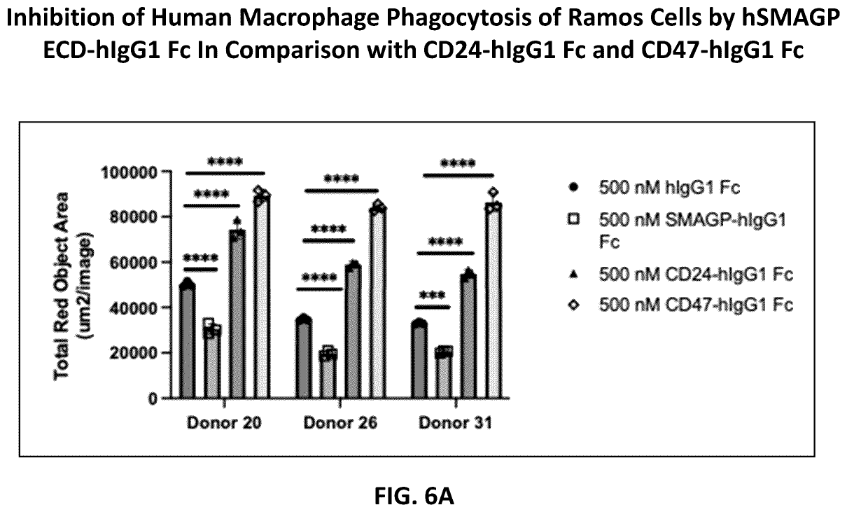

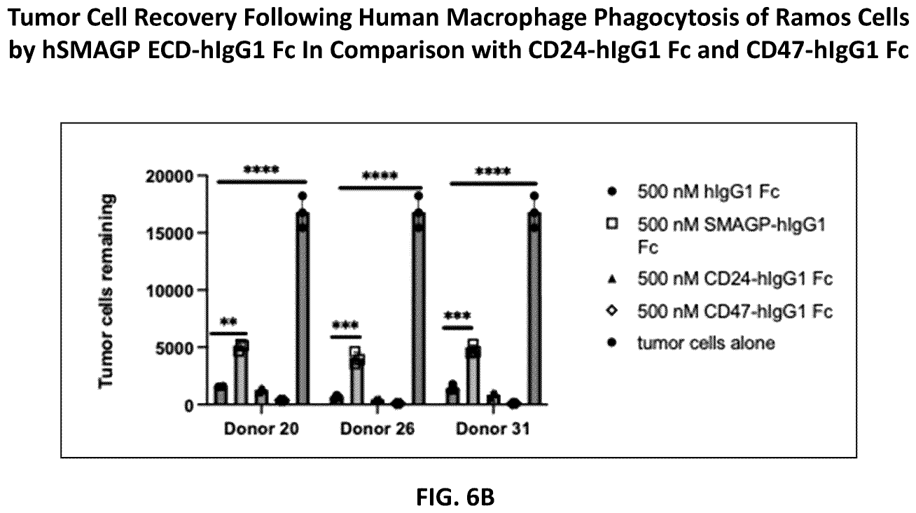

Human macrophages were generated from pan-monocytes isolated using negative selection from fresh peripheral blood leukapheresis products from healthy human donors. Monocytes were differentiated into macrophages by culturing for 7 days in the presence of recombinant human MCSF in RPMI media supplemented with 10% FBS. On Day 6, macrophages were detached and replated in MCSF media in 96 well plates and allowed to readhere overnight. On Day 7, Ramos tumor cells were collected, stained with pH sensitive dye pHrodo-Red-SE. Macrophages were stimulated with the hIgG1-Fc (Bio-X-Cell), hSMAGP ECD-hIgG1 Fc, CD24 ECD-hIgG1-Fc (R&D systems) or CD47 ECD-hIgG1-Fc (R&D systems) for approximately 45 minutes. After this preincubation, 0.5 μg/ml (FIG. 6A) or 1 μg/ml (FIG. 6B) anti-CD20 (rituximab biosimilar) was added to all macrophage wells and labeled tumor cells were added in an effector:target ratio of 1:2. Images were acquired via Incucyte at 3 hours post-coculture and total red object area corresponding to tumor cell phagocytosis was evaluated (FIG. 6A). Phagocytosis was allowed to occur for 24 hrs. and then all cells were detached and stained with anti-CD11b antibody and resuspended in FACS buffer, and 100 μl of this solution was acquired on an Attune flow cytometer. The amount of live CD11b cells in 100 μl was determined via analysis in FlowJo (FIG. 6B). One-way ANOVA was used to assess statistical significance in both FIG. 6A and FIG. 6B (p<0.05 (*), p<0.01 (**), p<0.001 (**), p<0.0001 (**).

As shown in FIG. 6A and FIG. 6B, hSMAGP ECD-hIgG1 Fc suppressed the phagocytosis of Ramos cells by human macrophages induced by anti-CD20 compared to isotype control as shown by a decrease in red area (FIG. 6A) at 3 hrs. and increased total number of tumor cells remaining at 24 hrs. (FIG. 6B). In comparison, CD24-hIgG1-Fc and CD47-hIgG1-FC significantly enhanced phagocytosis at 3 hrs. (FIG. 6A) and trended towards reduced amounts of tumor cells remaining at 24 hrs. (FIG. 6B). These results reveal that hSMAGP ECD-hIgG1 Fc decreases phagocytosis by two different measures and that this is not an intrinsic property of human Fc fusion proteins. Other “Don't Eat Me” signal protein ECDs from CD24 and CD47, each fused to hIgG1-Fc, enhanced phagocytosis, unlike hSMAGP ECD-hIgG1 Fc. These data underscore the surprising nature of the potent suppression of phagocytosis exhibited by hSMAGP ECD-hIgG1 Fc.

### Example 6. Inhibition of Human Macrophage Phagocytosis by hSMAGP ECD-hIgG1 Fc in the Presence of Rituximab

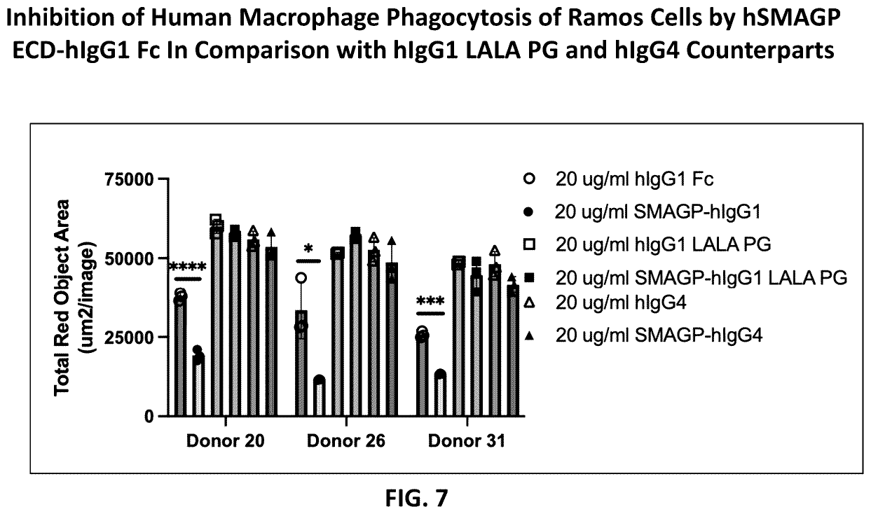

Human macrophages were generated and plated as in Example 5. On Day 7, Ramos tumor cells were collected and stained with pH sensitive dye pHrodo-Red-SE. Macrophages were stimulated with 20 μg/ml hIgG1-Fc (Bio-X-Cell), hSMAGP ECD-hIgG1 Fc, hIgG1 LALA PG (Bio-X-Cell), hIgG4 (Ichor Bio), hSMAGP ECD-hIgG1 LALA PG Fc, and hSMAGP ECD-hIgG4 for approximately 45 minutes. After this preincubation, 0.5 μg/ml anti-CD20 (rituximab biosimilar) was added to all macrophage wells and labeled tumor cells were added in an effector:target ratio of 1:2. Images were acquired via Incucyte at 3 hours post-coculture and total red object area corresponding to tumor cell phagocytosis was evaluated (FIG. 7). One-way ANOVA was used to assess statistical significance (p<0.05 (*), p<0.01 (**), p<0.001 (***), p<0.0001 (****)).

Intriguingly, only hSMAGP ECD-hIgG1 Fc, which includes a functional Fc domain, decreased phagocytosis compared to its matched isotype control. Fc-dead (hSMAGP-hIgG1 LALA PG) or hIgG4-fused variants of the hSMAGP ECD did not decrease phagocytosis. Based on these data, binding of the Fc domain to Fc receptors may be important for the suppression of phagocytosis by hSMAGP. Comparison to matched isotype control, as well as the data of Example 5, show that suppression of phagocytosis is not solely due to the Fc domain, as hSMAGP ECD-hIgG1 Fc suppresses phagocytosis compared to its matched isotype control, while other DEM signals fused to hIgG1 do not suppress phagocytosis. Taken together, these data highlight the uniqueness and potential therapeutic importance of hSMAGP ECD-hIgG1 Fc.

### Example 7. Inhibition of Human Macrophage Phagocytosis of Ramos Cells by hSMAGP ECD-hIgG1 Fc Across Macrophage Polarization States

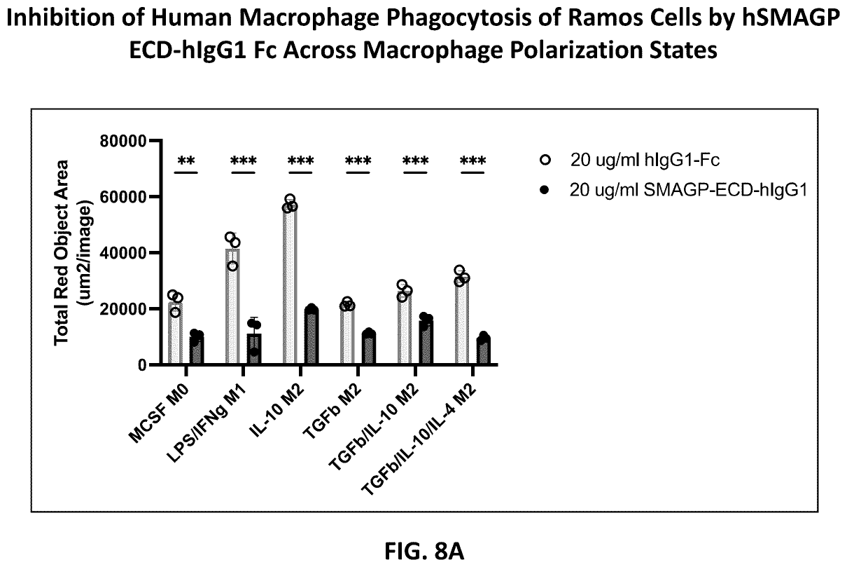

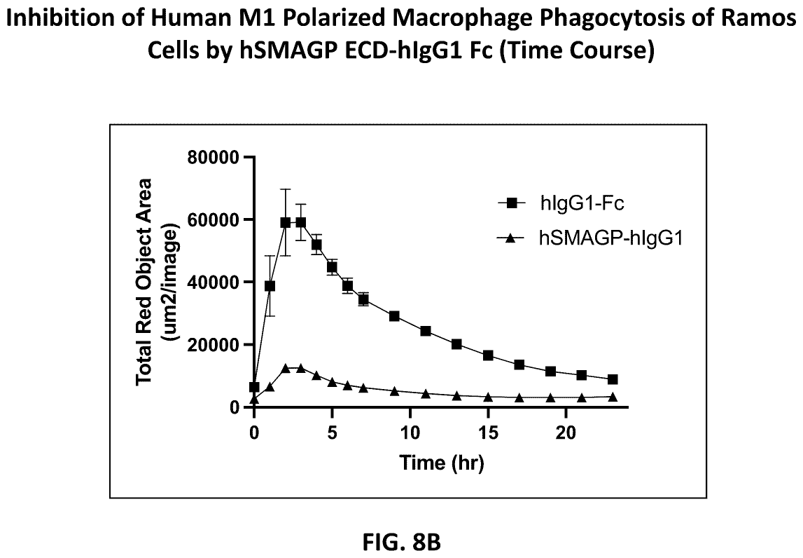

Human macrophages were generated from pan-monocytes isolated using negative selection from fresh peripheral blood leukapheresis products from healthy human donors. Monocytes were differentiated into macrophages by culturing for 7 days in the presence of recombinant human MCSF in RPMI media supplemented with 10% FBS. On day 6, macrophages were detached and replated in MCSF media in 96 well plates and allowed to re-adhere overnight in the presence of MCSF plus polarization stimulation as indicated (IFN-γ, LPS, TGF-β, IL-10, IL-4). LPS was purchased from Invivogen; all other polarization stimuli were purchased from R&D systems. On day 7, Ramos tumor cells were collected and stained with pH sensitive dye pHrodo-Red-SE. Macrophages were stimulated with the hIgG1-Fc or hSMAGP ECD-hIgG1 Fc for approximately 1 hr. hIgG1-Fc was purchased from Bio-X-Cell. After this preincubation, 10 μg/ml anti-CD47 antibody (B6H12, Bio-X-Cell) was added to all macrophage wells and labeled tumor cells were added (effector:target ratio of 1:2). Images were acquired via Incucyte for 21 hrs. The total red object area corresponding to tumor cell phagocytosis at 2 hrs. post coculture for each polarization condition (M0, without polarization stimulation; M1, induced by LPS and IFN-γ; M2, induced by IL-10; M2, induced by TGF-β; M2, induced by TGF-β and IL-10; and M2, induced by TGF-β, IL-10, and IL-4) treated with hSMAGP ECD-hIgG1 Fc or hIgG1 Fc control was determined (FIG. 8A). Multiple unpaired t-tests corrected for multiple comparisons were used to assess statistical significance (p<0.01 (**), p<0.001 (***)). A time course for phagocytosis of Ramos cells by M1 polarized macrophages (induced by LPS and IFN-γ) treated with hSMAGP ECD-hIgG1 Fc or hIgG1 Fc control in the presence of anti-CD47 antibody (B6H12) was determined over 21 hrs. (FIG. 8B).

As shown in FIG. 8A, hSMAGP ECD-hIgG1 Fc suppressed the phagocytosis of Ramos cells by human macrophages induced by anti-CD47 compared to isotype control as shown by significantly decreased red area in all macrophage polarization conditions tested. Strong suppression of phagocytosis by inflammatory M1 macrophages was observed across the entire time course of the assay (FIG. 8B). These results demonstrate that hSMAGP ECD-hIgG1 Fc is broadly able to suppress phagocytosis by macrophages across a range of polarization states/stimulation conditions. The suppression of phagocytosis in inflammatory M1 macrophages in particular suggests that hSMAGP ECD-hIgG1 Fc may be used to dampen phagocytosis under inflammatory conditions, e.g., diseases or disorders such as autoimmune disorders with excess phagocytosis that causes pathology.

### Example 8. Inhibition of Human Macrophage Phagocytosis of Opsonized Red Blood Cells by SMAGP ECD-hIgG1 Fc

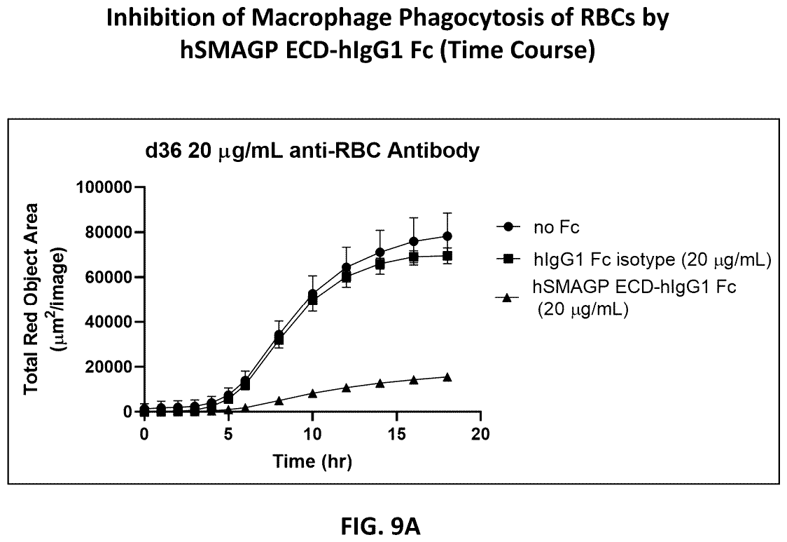

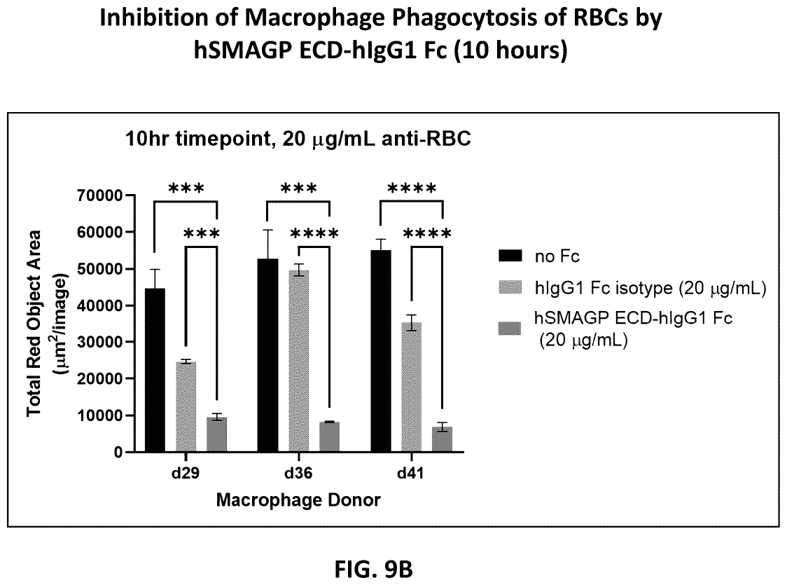

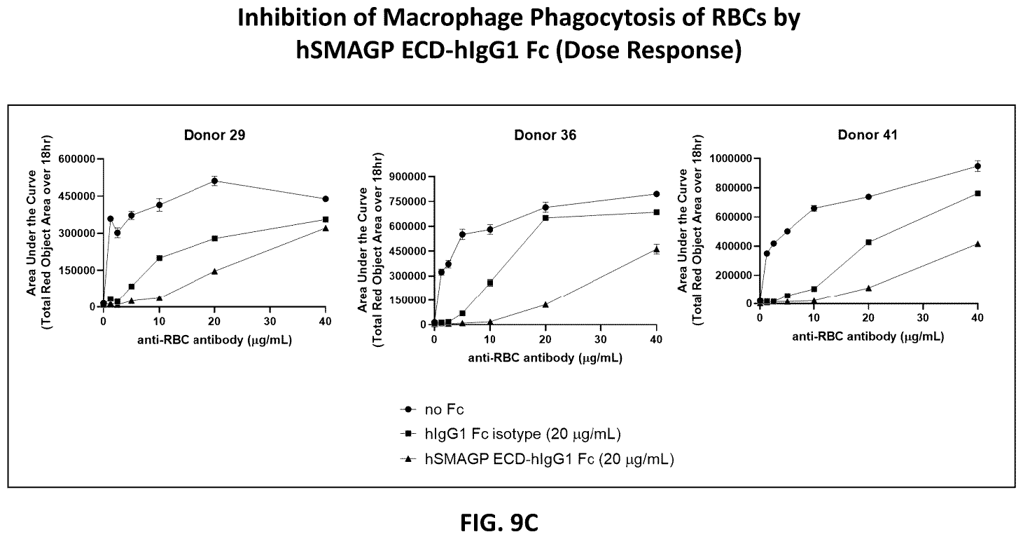

Human macrophages were generated from pan-monocytes isolated using negative selection from fresh peripheral blood leukapheresis products from healthy human donors. Monocytes were differentiated into macrophages by culturing for 7 days in the presence of recombinant human MCSF in RPMI media supplemented with 10% FBS. On day 6, macrophages were detached and replated in MCSF media in 96 well plates and allowed to readhere overnight. On day 7, red blood cells (RBCs) from pooled human donors (Rockland Immunochemicals) were washed and stained with pH sensitive dye pHrodo-Red-SE. Then RBCs were opsonized with polyclonal anti-RBC antibody (Rockland Immunochemicals) at 1.25, 2.5, 5, 10, 20, or 40 μg/mL, or no anti-RBC antibody, at 37° C. for 30 minutes. Macrophages were stimulated with 20 g/mL of hIgG1 Fc (Bio-X-Cell) or SMAGP ECD-hIgG1 Fc or media alone for 30 minutes while RBCs were being opsonized. Opsonized RBCs were washed and added to the macrophages at E:T of 1:5. Images were acquired via Incucyte every 1-2 hours post-coculture, and total red object area corresponding to RBC phagocytosis was evaluated for each time point. A time course for a representative donor at opsonizing antibody concentration of 20 μg/mL is shown in FIG. 9A. The total red object area at 10 hrs with an opsonizing antibody concentration of 20 μg/mL is shown for all three donors tested in FIG. 9B. Multiple unpaired t-tests were used to determine statistical significance (p<0.05 (*), p<0.01 (**), p<0.001 (***), p<0.0001 (****)). The total phagocytosis for each concentration of opsonizing antibody with or without hSMAGP ECD-hIgG1 Fc or hIgG1 Fc was quantified by calculating the area under the curve of total red object area for the time course and plotted versus opsonizing antibody concentration for each donor in FIG. 9C.

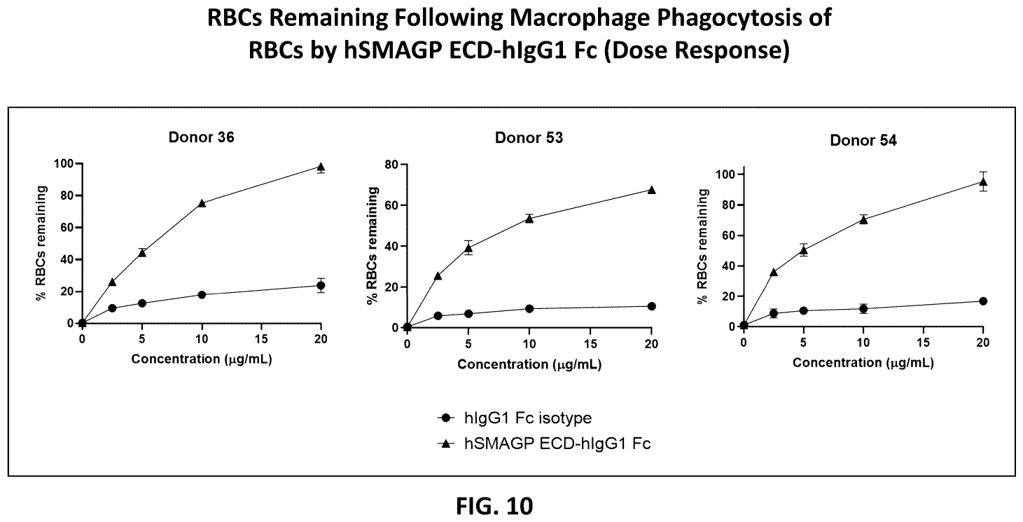

Next, human macrophages were generated as described in the previous paragraph of this Example. On day 7, RBCs from pooled human donors (Rockland Immunochemicals) were washed and stained with pH sensitive dye pHrodo-Red-SE and Cytopainter green. Then RBCs were opsonized with polyclonal anti-RBC antibody or isotype control (Rockland Immunochemicals) at 10 μg/mL at 37° C. for 30 minutes. Macrophages were stimulated with 2.5, 5, 10, or 20 μg/mL of cither hIgG1 Fc (Bio-X-Cell) or hSMAGP ECD-hIgG1 Fc, or media alone, for 30 minutes while RBCs were being opsonized. Opsonized RBCs were washed and added to the macrophages at E:T of 1:5 and Incubated overnight. Then all cells were detached and resuspended in FACS buffer, and 100 μL of this solution was acquired on an Attune flow cytometer. Macrophages were differentiated from RBCs by size. The quantity of live RBCs remaining in 100 μL was determined via analysis in FlowJo. The quantity of RBCs remaining was normalized to the quantity of RBCS remaining for RBCs opsonized with an isotype control antibody in the presence of macrophages from each donor. The percent RBCs remaining vs concentration of hSMAGP ECD-hIgG1 Fc or hIgG1 Fc is plotted in FIG. 10.

The results demonstrate that hSMAGP ECD-hIgG1 Fc is able to suppress the phagocytosis of RBCs by human macrophages induced by anti-RBC antibody compared to isotype hIgG1 Fc control, as shown both by decreased red object area (FIGS. 9A-9C) as well as by higher percentage of RBCs remaining (FIG. 10).

### Example 9. Macrophage Cytokine Expression in Presence and Absence of LPS Stimulation

Human macrophages were generated from monocytes isolated from fresh peripheral blood leukapheresis products from healthy human donors. Briefly, CD14⁺ monocytes were sorted via negative selection using Stem Cell Monocyte Enrichment kit. Monocytes were then cultured in RPMI with Glutamax+heat-inactivated fetal calf serum and MCSF for 5 days in tissue-culture treated plates. Cells were lifted and replated into 96 well plates in MCSF (M0) for two additional days. Cells were treated with either no Fc, hIgG1 Fc control (20 μg/ml), or hSMAGP ECD-hIgG1 Fc (20 μg/ml), plus or minus 100 ng/ml LPS overnight. The next day, plates were spun and supernatant was collected. The quantity of cytokine released was analyzed in multiplex by U-PLEX cytokine assay (Meso Scale Discovery) according to the manufacturer's instructions.

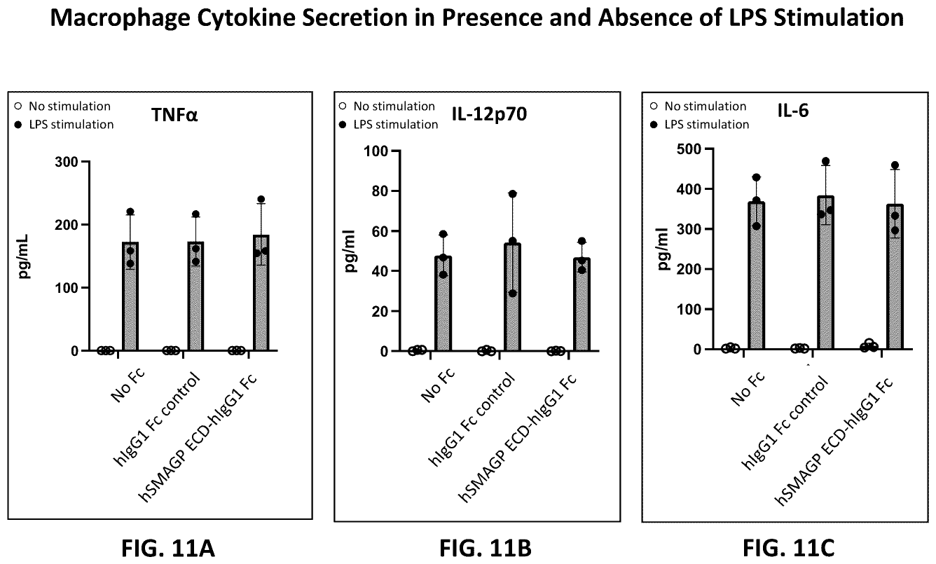

As shown in FIGS. 11A-11C, hSMAGP ECD-hIgG1 Fc did not significantly modulate release of TNFα (FIG. 11A), IL-12p70 (FIG. 11B), or IL-6 (FIG. 11C) either at steady state or in the presence of 100 ng/ml of LPS. Representative donor of three is shown in FIG. 11. A repeat of this study yielded similar results. These data suggest that hSMAGP ECD-hIgG1 Fc does not induce general enhanced inflammatory effects in macrophages under the conditions tested.

### Example 10. Identification and Characterization of CLEC10A as a Binding Partner for SMAGP

In order to identify a cognate cell-surface receptor to SMAGP that could potentially engage SMAGP and mediate signaling that would decrease macrophage phagocytosis of tumor cells, the binding interactions of hSMAGP ECD-hIgG1 Fc and cSMAGP ECD-hIgG1 Fc with a library of human lectins were evaluated. Lectins are a broad category of surface-expressed proteins that mediate various protein-protein and protein-carbohydrate interactions. A set of 37 recombinant human lectins was chosen based upon their reported expression and potential roles in immune cells. The lectins were assigned to a location and immobilized on multivalent N-hydroxysuccinimide (NHS) coated microarray slides to create the human recombinant lectin microarray. The custom microarrays were made in a 16-subarray format. In each subarray, four replicates were spotted for each human lectin, and also included a negative control, positive control, and marker.

The recombinant human lectin microarray assay utilized the accumulation of fluorescent signal on designated spots to identify the particular lectins to which a probe reagent may bind. The recombinant SMAGP-Fc fusions served as the probes and were prepared for the assay by first labeling with biotin, then hybridized to the array, followed by fluorescent streptavidin. Specifically, the hSMAGP ECD-hIgG1 Fc and cSMAGP ECD-hIgG1 Fc were labelled with EZ-link NHS-LC-LC-biotin, followed by overnight dialysis (in PBS, 10 kDa cutoff) and concentration (30 kDa cutoff). The biotinylated probes were then ready for hybridization to the microarray.

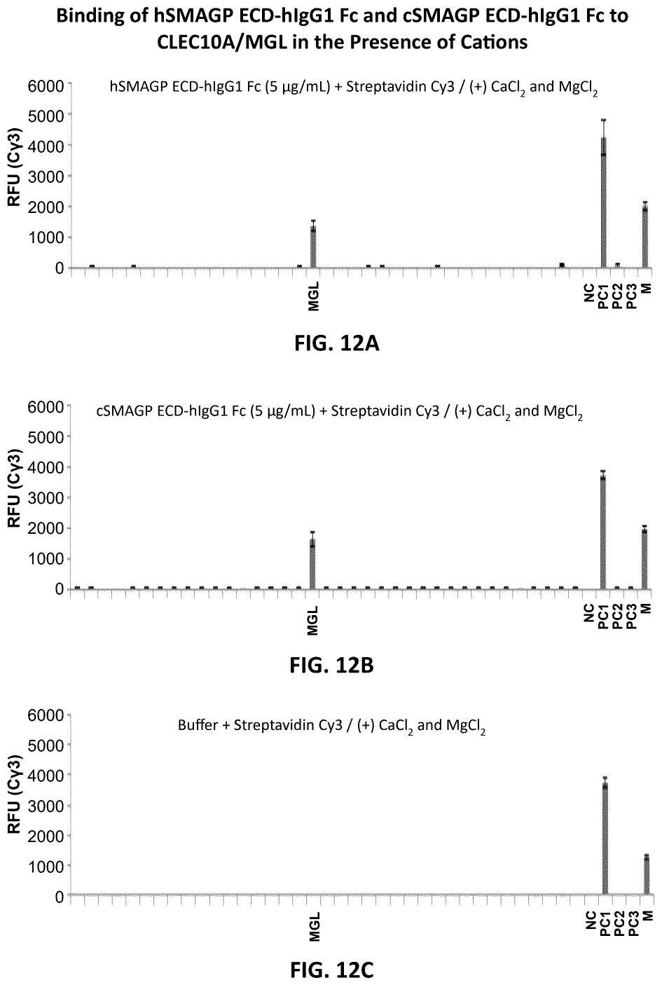

To determine whether hSMAGP ECD-hIgG1 Fc or cSMAGP ECD-hIgG1 Fc interact with any of the lectins, biotinylated versions were analyzed using the custom microarray. The microarray was pre-treated with buffer TBS-T (supplemented with 2 mM CaCl₂ and MgCl₂) at room temperature for 30 minutes. Then hSMAGP ECD-hIgG1 Fc or cSMAGP ECD-hIgG1 Fc (5 μg/ml) were added to the microarray and incubated at room temperature for 1 hour After incubation, the microarray was washed, and streptavidin-Cy3 (0.5 μg/ml) was added to the microarray. After incubation at room temperature for 1 hour, the microarray was washed and scanned at 532 nm using high laser intensity (1 PMT). Specific binding of only CLEC10A was observed for hSMAGP ECD-hIgG1 Fc (FIG. 12A) and cSMAGP ECD-hIgG1 Fc (FIG. 12B), while no binding was observed for the negative control consisting of buffer alone (FIG. 12C). As expected, PC1 (consisting of Amine-PEG-biotin) showed positive signal (FIGS. 12A-12C).

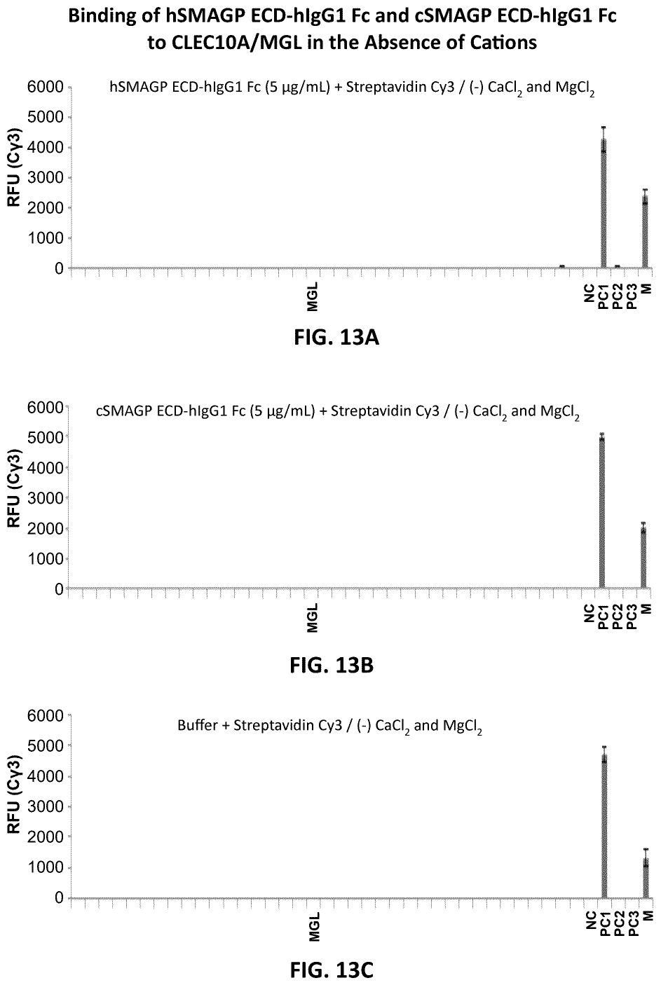

CLEC10A is a C-type lectin that requires calcium for efficient substrate binding. To test for binding specificity, the microarray binding assay was run in the absence of 2 mM CaCl₂) and MgCl₂. As expected, CLEC10A binding of hSMAGP ECD-hIgG1 Fc and cSMAGP ECD-hIgG1 Fc was lost in the absence of cations (FIGS. 13A-13C). Thus, CLEC10A binding of hSMAGP ECD-hIgG1 Fc and cSMAGP ECD-hIgG1 Fc is cation-dependent. Given that lectins generally lose their carbohydrate-binding activity when cations are removed, these data suggest that the observed binding is likely from a glycan-receptor interaction. Thus, glycans on hSMAGP ECD-hIgG1 Fc and cSMAGP ECD-hIgG1 Fc may create a multivalent scaffold for CLEC10A binding.

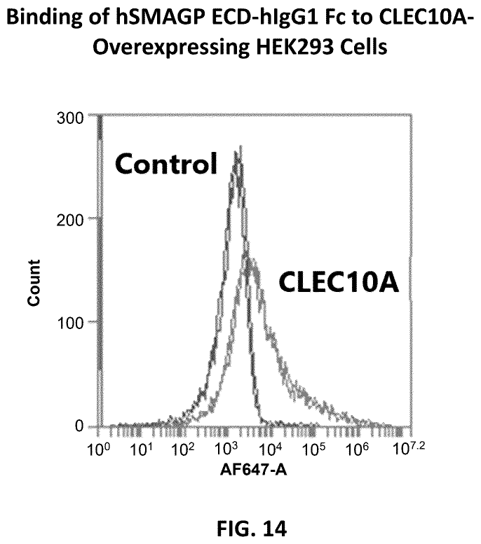

To test the binding of hSMAGP ECD-hIgG1 Fc to CLEC10A, HEK293 cells were transfected with expression vectors encoding ZsGreen1 only (Control) or ZsGreen1 and CLEC10A. Live cell transfectants, including Control-only transfected cells, were incubated with 5 μg/mL of hSMAGP ECD-hIgG1 Fc in a PBS+Ca2⁺/Mg2⁺ assay buffer. Cells were washed and the binding was detected using AF647-labeled anti-human Fc secondary antibody by flow cytometry. A 3.3-fold change in median fluorescence intensity, averaged over two replicates, was observed for CLEC10A expressing cells over the control (FIG. 14).

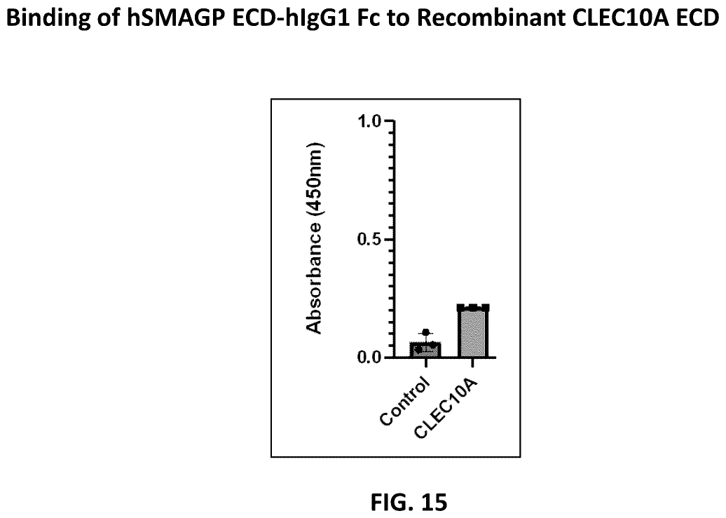

Binding of recombinant hSMAGP ECD-hIgG1 Fc to the recombinant CLEC10A ECD was evaluated in enzyme-linked immunosorbent assay (ELISA). Human CLEC10A (His-tagged extracellular domain) or human IgG1 Fc (control) proteins were immobilized to Corning 96-well clear flat bottom polystyrene high bind microplates at 4° C. overnight in PBS+2 mM Ca2⁺/Mg2⁺ buffer. Plates were blocked using 5% skim milk, followed by addition of biotinylated hSMAGP ECD-hIgG1 Fc protein (10 μg/ml) to the wells. Binding of biotinylated hSMAGP ECD-hIgG1 Fc to the immobilized proteins was detected using HRP-conjugated streptavidin. The signal was developed using TMB substrate. The assay was performed in triplicate. As shown in FIG. 15, binding of hSMAGP ECD-hIgG1 Fc to the CLEC10A ECD was observed.

In conclusion, CLEC10A was identified as a specific binding partner of hSMAGP ECD-hIgG1 Fc and cSMAGP ECD-hIgG1 Fc, with cation-dependent binding, through interrogation of a lectin library. Binding of hSMAGP ECD-hIgG1 Fc to CLEC10A was confirmed both in CLEC10A-overexpressing cells and in an ELISA assay with recombinant CLEC10A ECD.

### Example 11. Identification and Characterization of CD177 as Binding Partner for SMAGP

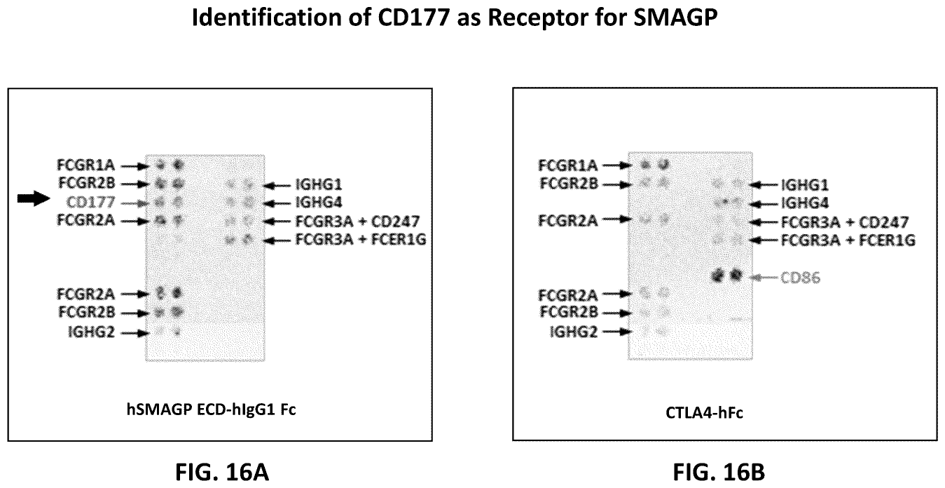

In order to identify one or more further cognate cell-surface receptors to SMAGP in addition to CLEC10A, hits from a library screen of over 6,000 full-length human plasma membrane proteins were identified, expressed in HEK293 cells, and probed with 5 μg/mL of hSMAGP ECD-hIgG1 Fc or 1 g/mL CTLA4-hFc (control). All expression vectors encoding the membrane proteins, along with a fluorescent expression control, were spotted in duplicate. Binding was detected using AF647 anti-hIgG Fc secondary antibody. Comparison of hits for SMAGP-hFc (FIG. 16A) and CTLA4-hFc (FIG. 16B) led to the identification of CD177 as a SMAGP-specific binding protein.

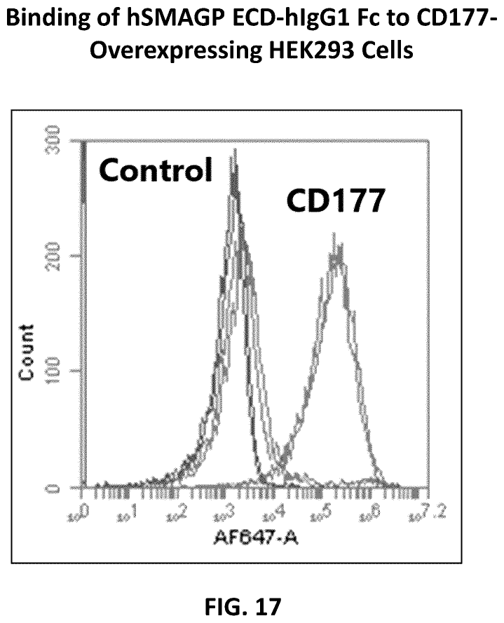

Binding of hSMAGP ECD-hIgG1 Fc to CD177 was reevaluated in a live cell assay using flow cytometry. HEK293 cells were transfected with expression vectors encoding ZsGreen1 only (Control) or ZsGreen1 and CD177. Live cell transfectants, including Control-only transfected cells, were incubated with 5 μg/mL of hSMAGP ECD-hIgG1 Fc. Cells were washed and the binding was detected using AF647-labeled anti-human Fc secondary antibody by flow cytometry. The assays were performed in duplicate. A 157-fold change in median fluorescence intensity was observed for CD177-expressing cells over the control (FIG. 17).

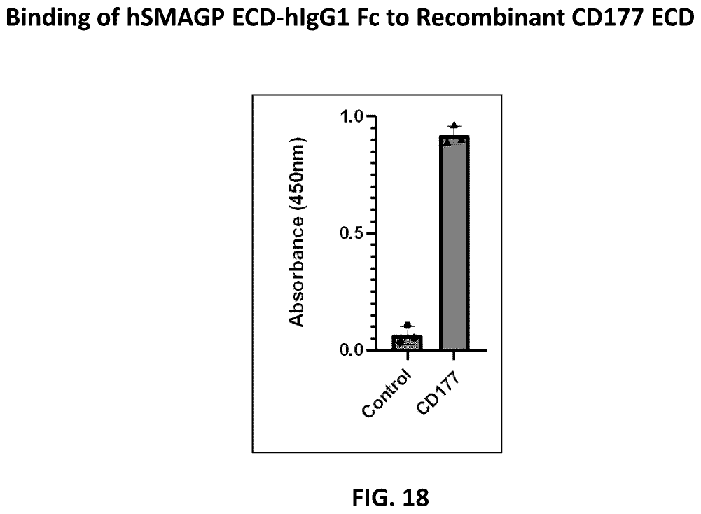

Binding of recombinant hSMAGP ECD-hIgG1 Fc to the recombinant CD177 ECD was evaluated in enzyme-linked immunosorbent assay (ELISA). Human CD177 (His-tagged extracellular domain) or human IgG1 Fc (control) proteins were immobilized to Corning 96-well clear flat bottom polystyrene high bind microplates at 4° C. overnight in PBS+2 mM Ca2⁺/Mg2⁺ buffer. Plates were blocked using 5% skim milk, followed by addition of biotinylated hSMAGP ECD-hIgG1 Fc protein (10 μg/ml) to the wells. Binding of biotinylated hSMAGP ECD-hIgG1 Fc to the immobilized proteins was detected using HRP-conjugated streptavidin. The signal was developed using TMB substrate. The assay was performed in triplicate. As shown in FIG. 18, binding of hSMAGP ECD-hIgG1 Fc to the CD177 ECD was observed.

To evaluate the binding of aglycosylated hSMAGP ECD to CD177, an hSMAGP ECD peptide (H2N-MTSLLTTPSPREELMTTPILQPTEALSPEDGASTALK-Biotin-amide (SEQ ID NO: 62)) was chemically synthesized. An extra lysine residue was added to the C terminus to facilitate biotinylation. Another peptide, with a scrambled hSMAGP ECD sequence (H2N-TRTTLIMESTTPALMLLEPLGSPQDAPTSTEEAPSLK-Biotin-amide (SEQ ID NO: 63)), was synthesized as a control. Peptide binding to the recombinant CD177 ECD-Fc (CD177 extracellular domain with Fc tag) or hIgG1-Fc (IgG1 domain with Fc tag) negative control was evaluated in an enzyme-linked immunosorbent assay (ELISA). CD177 ECD-Fc and hIgG1-Fc were immobilized to separate Corning 96-well Clear Flat Bottom Polystyrene High Bind Microplates at 4° C. overnight in PBS. Plates were blocked using 5% skim milk. Biotinylated hSMAGP ECD peptide or the scrambled control (10 μg/ml) was pre-incubated with HRP-conjugated streptavidin (5 μg/ml) and added to the plates. In a control assay, an anti-CD177 antibody (Biolegend Cat #315802; 10 μg/ml) was premixed with the biotinylated SMAGP ECD peptide. The signal was developed using TMB substrate. The assay was performed in triplicate.

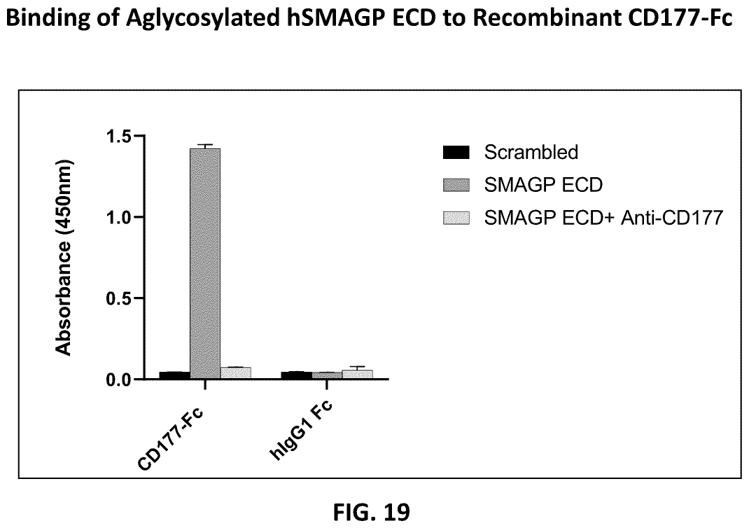

As shown in FIG. 19, the hSMAGP ECD peptide exhibited binding to CD177-Fc, while the scrambled hSMAGP ECD peptide and the hSMAGP ECD peptide plus anti-CD177 antibody did not exhibit binding. Importantly, this result revealed that the interaction of the hSMAGP ECD with the CD177 ECD does not require glycosylation of the hSMAGP ECD. Accordingly, the hSMAGP ECD need not be recombinantly expressed in order to bind the CD177 ECD.

In conclusion, CD 177 was identified as a specific binding partner of hSMAGP ECD-hIgG1 Fc through interrogation of a human plasma membrane protein library. Binding of hSMAGP ECD-hIgG1 Fc to CD177 was confirmed both in CD177-overexpressing cells and in an ELISA assay with recombinant CD177 ECD, and binding of aglycosylated hSMAGP ECD peptide to CD177 was confirmed in another ELISA assay with recombinant CD177 ECD.

### Example 12. CD177 and CLEC10A Expression on Various Cell Types

Human macrophages were generated from monocytes isolated from fresh peripheral blood leukapheresis products from healthy human donors. Briefly, CD14⁺ monocytes were sorted via negative selection using Stem Cell Monocyte Enrichment kit. Monocytes were then cultured in RPMI with Glutamax+heat-inactivated fetal calf serum and MCSF for 5 days in tissue-culture treated plates. Cells were lifted and replated in either MCSF (M0) or MCSF+IL-4, IL-10 and TGF-β (M2a/c) for two additional days. Cells were Fc-blocked and then stained with anti-CD177, anti-CLEC10A or isotype controls and a viability dye. Cells were washed and run on the flow cytometer.

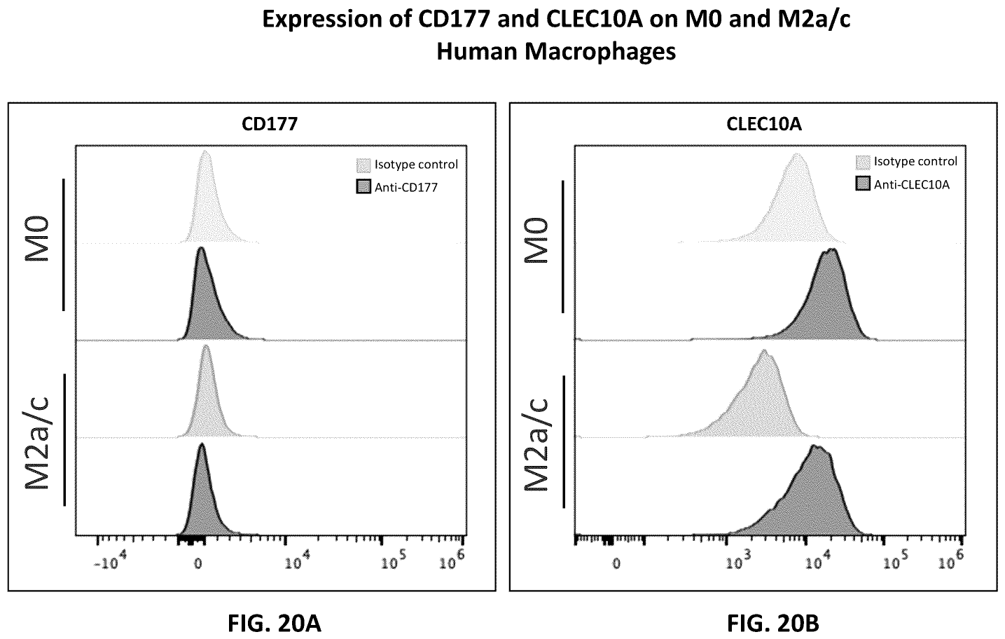

As shown in FIG. 20A, no CD177 expression was detected on either M0 or M2a/c human macrophages. As shown in FIG. 20B, CLEC10A expression was detected on both M0 and M2a/c human macrophages. Representative donor of three shown.

In a further experiment, whole blood from healthy human donors was obtained. Red blood cells were lysed and then cells were Fc-blocked. Peripheral blood cells were stained with lineage markers (CD3, CD56, CD14, CD19 and CD15), viability dye, and either anti-CD177, anti-CLEC10A or isotype controls. Cells were washed and run on the flow cytometer.

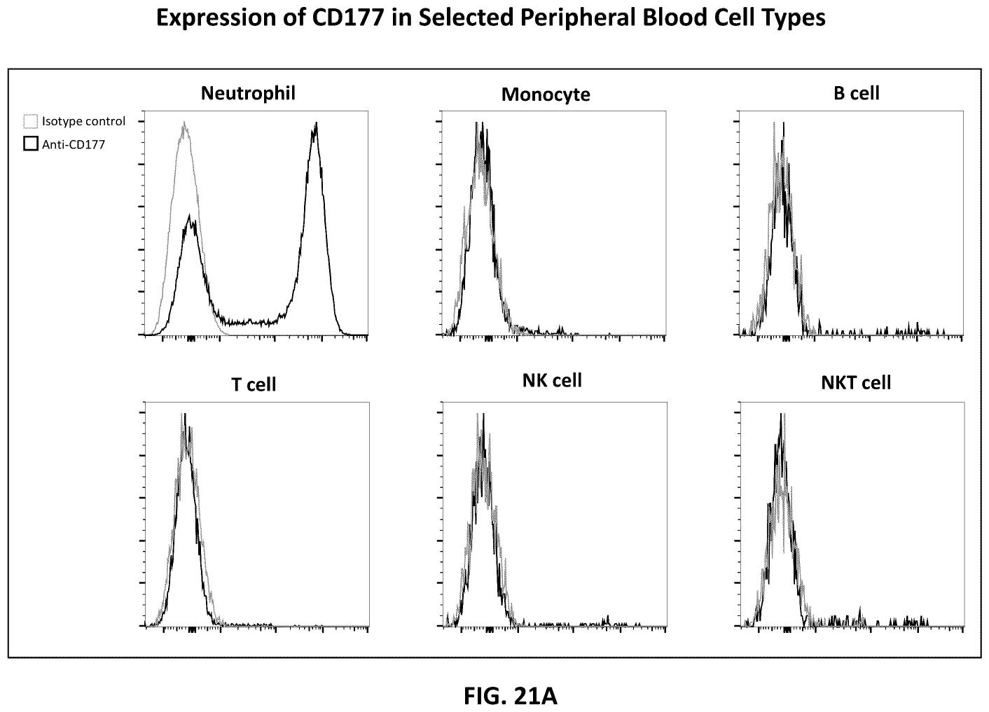

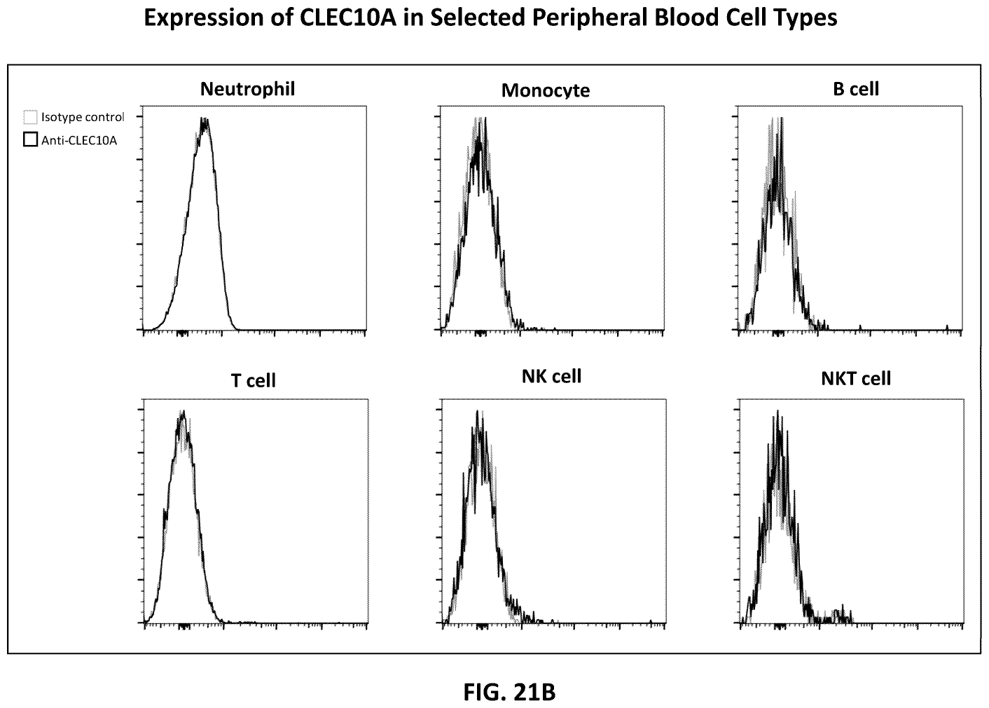

As shown in FIG. 21A, peripheral blood neutrophils (CD15+) were found to strongly express CD177. CD177 expression was not detected in peripheral blood monocytes (CD14+, CD15−), B cells (CD19+), T cells (CD3+CD56−), NK cells (CD56+), or NKT cells (CD56+CD3+). As shown in FIG. 21B, CLEC10A expression was not detected on any of the peripheral blood cell types tested.

### Example 13. Evaluation of SMAGP ECD-Fc Fusion Molecules in Mouse Model of ITP

Immune Thrombocytopenia (ITP) is an autoimmune disorder manifested by a decrease in the number of platelets in the blood due to autoantibodies to platelet membrane antigens, resulting in splenic sequestration and phagocytosis by macrophages. ITP is associated with bleeding disorders ranging from mild to fatal. ITP is usually treated by corticosteroids, intravenous immunoglobin, anti-D immunoglobin or immunosuppressive medications. The benefit of these treatments can be transient, and some patients may require platelet transfusion or splenectomy. Therefore, novel treatments, particularly those that may reduce phagocyte activity, are needed for ITP patients.

SMAGP ECD-mouse Fc fusion molecules, including wild-type and mutant variants (including variants with mutations in the Fc domain), are evaluated in an art-recognized mouse model of ITP (see, e.g., Samuelsson et al. (2001) Science 291 (5503): 484-486, and Huang et al. (2010) Blood 116 (23): 5002-5009, each of which is hereby incorporated by reference in its entirety). ITP is induced by intravenous administration of 0.1 mg/kg body weight of anti-platelet (PLT) monoclonal antibody (mAb, rat anti-mouse integrin αIIb/CD41 Ig, clone MWReg30; BD Biosciences). To analyze PLT counts, whole blood samples (50-100 μL) from mice are collected from the retro-orbital venous plexus and mixed with anticoagulant ACD solution (38 mM citric acid, 75 mM sodium citrate, 100 mM dextrose) in Eppendorf tubes. PLT counts are then measured with a hematology analyzer (KX-21N; Sysmex) at 0, 2, 4, and 24 hours after anti-CD41 Ig MWReg30 treatments. To investigate the effect of SMAGP ECD-Fc fusion molecules on ITP, the mice are intravenously or intraperitoneally treated with 1-50 mg/kg SMAGP ECD-Fc fusion molecules or vehicle, 10-120 minutes before ITP induction. IVIg (1 g/kg) is used as a positive control to reduce the deleterious effect of CD41 Ig on PLT counts.

### Example 14. Evaluation of SMAGP ECD-Fc Fusion Molecules in Mouse Models of HLH

Hemophagocytic lymphohistiocytosis (HLH) is a life-threatening syndrome of overwhelming immune activation most often affecting infants and young children. HLH causes may be primary and linked to inherited mutation in specific genes, such as the perforin 1 (PRF1) gene. Macrophage activation syndrome (MAS) is the term used for a type of secondary HLH that occurs in people with an autoimmune or autoinflammatory disease. The diseases most commonly associated with MAS are juvenile systemic arthritis, adult-onset Still's disease, and systemic lupus erythematosus. HLH is characterized by overt activation of macrophages, production of cytokines, and phagocytosis, causing excessive inflammation and tissue destruction. HLH is usually treated by steroids and chemotherapy. Treatments that inhibit macrophage activity and are better tolerated are needed for HLH patients.

SMAGP ECD-mouse Fc fusion molecules, including wild-type and mutant variants (including variants with mutations in the Fc domain), are evaluated in an art-recognized mouse model of primary HLH (see, e.g., Jordan et al. (2004) Blood 104 (3): 735-743, and Huang et al. (2017) Haematologica 102 (11): 1956-1968, each of which is hereby incorporated by reference in its entirety). Perforin 1 (PRF1)-deficient mice manifest all the features of HLH after infection with lymphocytic choriomeningitic virus (LCMV). Following LCMV infection, perforin-deficient mice develop fever, splenomegaly, pancytopenia, hypertriglyceridemia, hypofibrinogencmia, and elevation of multiple serum cytokine levels, and hemophagocytosis is evident in many tissues. C57BL/6-Prf1tm1Sdz (pfp−/−) mice are intravenously or intraperitoneally injected with 5e¹ to 10e⁴ particle-forming units (PFU) of LCMV. Mice are sacrificed and analyzed on day 8-14 after inoculation. Spleen weight, hemoglobin (Hb), platelet (PLT) and neutrophils in blood, CD80 and CD80 expression on splenic macrophages are assessed as symptoms of HLH. Mice survival beyond day 20 is also assessed. To investigate the effect of SMAGP ECD-Fc fusion molecules on HLH pathology, the mice are intravenously or intraperitoneally treated with 1-50 mg/kg SMAP fusion proteins or vehicle every 2-3 days before and after LCMV injection.

The repeated CpG-treated mouse is a model of secondary HLH (see, e.g., Behrens et al. (2011) J Clin Invest. 121 (6): 2264-2277; Das et al. (2016) Blood 127 (13): 1666-1675; and Huang et al. (2017) Haematologica 102 (11): 1956-1968, each of which is hereby incorporated by reference in its entirety). C57BL/6 mice are injected with 50-75 μg of CpG DNA on days 0, 2, 4, 6, and 8 as described. Mice are euthanized and organs collected for analysis on day 9 for spleen size and cellularity, blood hemoglobin and platelet number. The effects of SMAGP ECD-Fc fusion molecules on secondary HLH pathology are assessed by intravenous or intraperitoneal treatment every 2 days with 1-50 mg/kg SMAGP ECD-Fc fusion molecules or vehicle before and during CpG administration.

### Example 15. SMAGP ECD-Lipid Nanoparticle Fusions

The SMAGP ECD, e.g., glycosylated or aglycosylated SMAGP ECD, is able to bind to leukocytes, e.g., myeloid cells, macrophages (e.g., M0 macrophages, M1 macrophages, M2 macrophages, M2a macrophages, M2b macrophages, M2c macrophages, or M2d macrophages), neutrophils, Kupffer cells, histiocytes, microglia, osteoclasts, dendritic cells, mast cells, regulatory T cells, tumor-infiltrating regulatory T cells, and granulocytes. Conjugation of SMAGP ECD to a nanoparticle encapsulating a payload, such as an mRNA, a circular RNA (cRNA), a tRNA, a miRNA, a siRNA, a sgRNA, an antisense oligonucleotide, a peptide, a virus, a viral DNA genome, a viral RNA genome, a vector, a DNA construct, a plasmid, or a drug may facilitate the delivery of the payload to leukocytes, thereby modulating the function of such leukocytes.

Delivery of nucleic acids (e.g., RNA or DNA) is particularly challenging, as cellular plasma membranes are not permeable to highly hydrophilic molecules. Nanoparticles, including lipid nanoparticles (LNP) formulations, have been demonstrated to be highly capable of delivering nucleic acid to cells. One such application has been the delivery of siRNA (e.g., Onpattro® (patisiran), Alnylam; see, e.g., Akinc et al. (2019) Nat Nanotechnol. 14 (12): 1084-1087, which is hereby incorporated by reference in its entirety) or mRNA (e.g., encoding the spike protein of SARS-COV-2, BioNtech/Pfizer and Moderna). Lipid nanoparticles are generally composed of four components: an ionizable lipid enabling endosomal escape; a pegylated lipid to enhance half-life in circulation (e.g., 1,2-dimyristoyl-rac-glycero-3-methoxypolyethylene glycol-2000 (DMG-PEG (2000)); or 2-[(polyethylene glycol)-2000]-N,N-ditetradecylacetamide (ALC-0159)) (e.g., from Avanti Polar Lipids); a helper lipid (e.g., 1,2-Distearoyl-sn-glycero-3-phosphorylethanolamine (DSPE); 1,2-distearoyl-sn-glycero-3-phosphocholine (DSPC); 1,2-dioleoyl-sn-glycero-3-phosphoethanolamine (DOPE); or 1,2-dioleoyl-sn-glycero-3-phosphoethanolamine-N-dibenzocyclooctyl(DBCO PE)) (e.g., from Avanti Polar lipids); and cholesterol (or derivative thereof). These individual components may be chemically conjugated to a SMAGP ECD to selectively deliver payloads to leukocytes, including macrophages and dendritic cells, to enhance antigen presentation for vaccines against infectious disease or cancer (e.g., SARS-CoV-2 vaccine or cancer vaccine) (see, e.g., Sahin et al. (2020) Nature 585 (7823): 107-112), or to enhance the tumoricidal activity of macrophage against tumor cells (e.g., by expressing IRF5 mRNA) (see, e.g. Zhang et al. (2019) Nature Communications 10:3974, which is hereby incorporated by reference in its entirety), or the tolerogenic activity for the treatment of autoimmune diseases (see, e.g., Krienke et al. (2021) Science 371 (6525): 145-153, which is hereby incorporated by reference in its entirety).

SMAGP ECD conjugation to helper lipid DSPE or PEG-lipid may occur at the amino or carboxy termini of SMAGP protein utilizing bioorthogonal/click chemistry (see, e.g., Simon et al. (2012) Bioconjugate Chem. 23 (2): 279-286, which is hereby incorporated by reference in its entirety).

The click chemistry can be carried out in either orientation, since both the non-natural Met analogues azidohomoalanine (Aha) and homopropargylglycine (Hpg) can be efficiently introduced, simply by exchanging the growth medium shortly before induction and by using a methionine-auxotrophic E. coli strain. The resulting “clickable” SMAGP ECD conjugates can be expressed in high amounts and are capable of biorthogonal coupling to “clickable” substrates containing the corresponding azides or alkynes for Cu(I)-dependent coupling or substituted cyclo-octynes for Cu(I)-independent click chemistry, respectively.

For N-terminal coupling, e.g., to N-terminal azidohomoalanine of the SMAGP ECD, the clickable DARPin Aha-Ec1 is reacted with a 2-fold molar excess of DBCO-PE at 4° C. and aliquots are taken over time, snap frozen, and stored at −20° C. SMAGP ECD conjugated to DBCO helper lipid may then be used in the nanoprecipitation of the payload LNP component reaction.

Since the human SMAGP ECD is devoid of any cysteine, a single C-terminal cysteine can be introduced for maleimide coupling to maleimide 1,2-distearoyl-sn-glycero-3-phosphoethanolamine-N-[maleimide(polyethylene glycol)-2000] (DSPE-PEG (2000)), or the PEG5000 analog thereof, from Avanti Polar lipids, to generate the SMAGP ECD conjugated to PEG. SMAGP engineered with a C-terminal cysteine is mixed with a 2-fold molar excess of DSPE-PEG (2000) or (5000) from a 5 mM stock in PBS and vortexed. The click reaction is performed at 4° C. for 72 hours in PBS pH 7.2 without agitation, and PEGylated SMAGP is separated from free DSPE-PEG and non-conjugated SMAGP using anion exchange chromatography. Conjugation may occur on DSPE-PEG prior to nanoprecipitation of the payload and LNP components or after LNP-payload has been formulated.

The invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims.

All references (e.g., publications or patents or patent applications) cited herein are incorporated herein by reference in their entireties and for all purposes to the same extent as if each individual reference (e.g., publication or patent or patent application) was specifically and individually indicated to be incorporated by reference in its entirety for all purposes. Other embodiments are within the following claims.

1.-164. (canceled)

165. A fusion molecule comprising a SMAGP extracellular domain (ECD) and one or more of a half-life extending moiety, a payload molecule, and a targeting moiety that binds to a target molecule on a cell.

166. The fusion molecule of claim 165, wherein the SMAGP ECD:
(a) is a wild-type human, mouse, or cynomolgus SMAGP ECD;
(b) comprises an amino acid sequence at least 95% identical to an amino acid sequence selected from the group consisting of SEQ ID NO: 1, 2, and 3;
(c) comprises an amino acid sequence selected from the group consisting of SEQ ID NO: 1, 2, and 3, or a variant thereof comprising 1-5 amino acid changes; or
(d) comprises an amino acid sequence selected from the group consisting of SEQ ID NO: 1, 2, and 3.

167. The fusion molecule of claim 165, wherein the half-life extending moiety:
(a) is an Fc region, optionally wherein the Fc region:
(i) is selected from the group consisting of human IgG1, IgG2, IgG3, IgG4, IgA1, and IgA2 Fc region;
(ii) is a human IgG1 Fc region comprising D265A and N297A mutations, numbered according to the EU numbering system;
(iii) is a human IgG1 Fc region comprising L234A, L235A, and P329G mutations, numbered according to the EU numbering system;
(iv) is a human IgG1 Fc region comprising S239D and 1332E mutations, numbered according to the EU numbering system;
(v) is a human IgG4 Fc region comprising an S228P mutation, numbered according to the EU numbering system;
(vi) is a mouse IgG2a Fc region;
(vii) is a mouse IgG2a Fc region comprising L234A, L235A, and P329G mutations, numbered according to the EU numbering system; or
(viii) comprises an amino acid sequence selected from the group consisting of SEQ ID NOs: 5-18 and 50-57; or
(b) is selected from the group consisting of human serum albumin, a serum albumin-binding moiety, and polyethylene glycol (PEG), optionally wherein the serum albumin-binding moiety is an antibody or antigen-binding portion thereof, further optionally wherein the serum albumin-binding moiety is a VH or VHH.

168. The fusion molecule of claim 165, wherein the SMAGP ECD is covalently linked to the half-life extending moiety, the payload molecule, and/or the targeting moiety via a linker, wherein the linker:
(a) is a peptide linker;
(b) is an Fc hinge region or portion thereof;
(c) is a human Fc hinge region or portion thereof;
(d) is a human IgG1, IgG2, IgG3, or IgG4 hinge region or portion thereof; and/or
(e) comprises an amino acid sequence selected from the group consisting of SEQ ID NOs: 4, 35-49, 58-61, or a variant thereof comprising 1-5 amino acid changes.

169. The fusion molecule of claim 165, wherein the half-life extending moiety, the payload molecule, or the targeting moiety is linked to the C-terminus of the SMAGP ECD or the N-terminus of the SMAGP ECD.

170. The fusion molecule of claim 165, wherein:
(a) the cell is selected from the group consisting of a diseased cell, a senescent cell, a cancer cell, a B cell, a T cell, and a dendritic cell;
(b) the target molecule is selected from the group consisting of DEP1, NTAL, EBP50, STX4, VAMP3, ARMCX3, B2MG, LANCL1, PLD3, VPS26A, DPP4, SCAMP4, MICA/B, TNFRSF10D/CD264, NOTCH1, NOTCH3, CD36, oxidized Vimentin, ICAM-1, uPAR, DEP1/PTPRJ/CD148, CD264, TNFRSF10D, TRAILR4, and CD26, optionally wherein the cell is selected from a diseased cell and a senescent cell;
(c) the target molecule is selected from the group consisting of ADAM9, B7-H3/CD276, BCMA, CA6, CA9, CD19, CD20, CD22, CD30, CD33, CD37, CD38, CD70, CD79b, CD123, CD138, CD157/BST1, P-cadherin CDH3, CEACAM5, CEACAM6, CLDN6, CLDN18.2, DLL3, EGFR, EGFRvIII, ENPP3, ENTPD2, EpCAM, FGR3, FLT3, FOLR1, GPA33, GPC3, GPNMB, GPRC5D, GUCY2C, Her2, HHLA2, LAMP1, SLC39A6/Liv-1, Mesothelin, MUC16/CA125, MUC17, SLC34A1/NaPi2a, Nectin 4, CD274/PD-L1, PSCA, PSMA/FOLH1, PVR, PVRIg, ROR1, SLITRK6, SSTR2, STEAP1, TROP2, and TMEM97, optionally wherein the cell is a cancer cell, optionally wherein the targeting moiety is rituximab or an antigen-binding fragment thereof; or
(d) the target molecule is a B cell surface antigen, optionally wherein the targeting moiety is rituximab or an antigen-binding fragment thereof, and
in each of (a) through (d), optionally wherein the targeting moiety is an antibody or antigen-binding fragment thereof.

171. The fusion molecule of claim 165, wherein the payload molecule:
(a) is selected from the group consisting of an mRNA, an miRNA, a cRNA, a tRNA, an siRNA, an sgRNA, an antisense oligonucleotide, a peptide, a virus, a viral RNA genome, a viral DNA genome, a vector, a plasmid, a DNA, a radionuclide, and a drug, optionally wherein the virus is an adeno-associated virus or a lentivirus or the vector is a DNA vector;
(b) modulates the activity of a cell expressing CLEC10A or CD177;
(c) is designed or selected to modulate the activity of a neutrophil;
(d) is selected from the group consisting of a small molecule drug that inhibits the NADPH oxidase complex responsible for the production of ROS, a degranulation inhibitor, and an enhancer of neutrophil apoptosis, optionally wherein:
(i) the small molecule drug that inhibits the NADPH oxidase complex responsible for the production of ROS is selected from the group consisting of diphenyleneiodonium and stelazine;
(ii) the degranulation inhibitor is nexinhib-20; and
(iii) the enhancer of neutrophil apoptosis is roscovitine;
(e) is a nucleic acid molecule encoding a chimeric antigen receptor for a neutrophil; and/or
(f) is enclosed in or attached to a nanoparticle, optionally wherein the nanoparticle is a lipid nanoparticle and the linker is linked to a lipid component of the lipid nanoparticle, further optionally wherein the lipid component of the lipid nanoparticle to which the linker is linked is selected from the group consisting of an ionizable lipid, a pegylated lipid, a helper lipid, and cholesterol, further optionally wherein the helper lipid is selected from the group consisting of 1,2-Distearoyl-sn-glycero-3-phosphorylethanolamine (DSPE); 1,2-distearoyl-sn-glycero-3-phosphocholine (DSPC); 1,2-dioleoyl-sn-glycero-3-phosphoethanolamine (DOPE); and 1,2-dioleoyl-sn-glycero-3-phosphoethanolamine-N-dibenzocyclooctyl (DBCO PE), further optionally wherein the helper lipid is DBCO PE, wherein the SMAGP ECD comprises an N-terminal azidohomoalanine, and wherein the linker comprises a clickable DARPin that binds to the DBCO PE and the N-terminal azidohomoalanine, further optionally wherein the pegylated lipid is selected from the group consisting of 1,2-distearoyl-sn-glycero-3-phosphoethanolamine-N-[maleimide(polyethylene glycol)-2000] (DSPE-PEG (2000) Maleimide); 1,2-distearoyl-sn-glycero-3-phosphoethanolamine-N-[maleimide(polyethylene glycol)-5000] (DSPE-PEG (5000) Maleimide); 1,2-dimyristoyl-rac-glycero-3-methoxypolyethylene glycol-2000 (DMG-PEG (2000)); and 2-[(polyethylene glycol)-2000]-N,N-ditetradecylacetamide (ALC-0159), further optionally wherein the pegylated lipid is DSPE-PEG (2000) Maleimide or DSPE-PEG (5000) Maleimide, and wherein the linker comprises a cysteine fused to the C terminus of the SMAGP ECD that binds to the pegylated lipid.

172. The fusion molecule of claim 165, wherein:
(a) the fusion molecule binds to a SMAGP ECD receptor on a cell,
(b) the SMAGP ECD is glycosylated or aglycosylated; or
(c) the fusion molecule further comprises a detectable label, optionally wherein the detectable label is selected from the group consisting of an enzyme, a fluorescent label, and a radioisotope.

173. The fusion molecule of claim 165, comprising:
(a) an amino acid sequence at least 95% identical to an amino acid sequence selected from the group consisting of SEQ ID NOs: 19-34;
(b) an amino acid sequence selected from the group consisting of SEQ ID NOs: 19-34; or
(c) a dimer of two polypeptides, each polypeptide comprising an amino acid sequence selected from the group consisting of SEQ ID NOs: 19-34.

174. A polynucleotide comprising a nucleotide sequence encoding the fusion molecule of claim 165, optionally wherein the polynucleotide is an mRNA.

175. An expression vector comprising the polynucleotide of claim 174.

176. A recombinant host cell comprising the polynucleotide of claim 174.

177. A nanoparticle comprising the fusion molecule of claim 165, a polynucleotide comprising a nucleotide sequence encoding the fusion molecule, or an expression vector comprising the polynucleotide, optionally wherein the nanoparticle is a lipid nanoparticle, further optionally wherein the polynucleotide is an mRNA.

178. A method of producing a fusion molecule, the method comprising culturing the recombinant host cell of claim 176 under conditions such that the fusion molecule is produced.

179. A composition comprising the fusion molecule of claim 165, a polynucleotide comprising a nucleotide sequence encoding the fusion molecule, an expression vector comprising the polynucleotide, or a nanoparticle comprising the fusion molecule, the polynucleotide, or the expression vector,
and a pharmaceutically acceptable carrier or excipient.

180. A method of modifying an activity of a leukocyte comprising contacting the leukocyte with the fusion molecule of claim 165, optionally wherein the leukocyte is selected from the group consisting of myeloid cells, macrophages, Kupffer cells, histiocytes, microglia, osteoclasts, dendritic cells, mast cells, neutrophils, regulatory T cells, tumor-infiltrating regulatory T cells, and granulocytes, optionally wherein the macrophage is selected from the group consisting of an M0 macrophage, an M1 macrophage, an M2 macrophage, an M2a macrophage, an M2b macrophage, an M2c macrophage, and an M2d macrophage, optionally wherein the activity comprises one or more of phagocytosis, cytokine production, chemokine production, antigen presentation, growth factor production, and protease production, optionally wherein the activity comprises phagocytosis of a population of cells or cell-like structures, further optionally wherein the population of cells or cell-like structures is selected from the group consisting of cancer cells, immune cells, neurons, red blood cells, and platelets, optionally wherein the activity of the leukocyte is increased or reduced.

181. A method of treating a disease or disorder in a subject in need thereof, comprising administering to the subject a therapeutically effective amount of the fusion molecule of claim 165, a polynucleotide comprising a nucleotide sequence encoding the fusion molecule, an expression vector comprising the polynucleotide, a nanoparticle comprising the fusion molecule, the polynucleotide, the expression vector, or a composition comprising any of the foregoing and a pharmaceutically acceptable carrier or excipient, optionally wherein the disease or disorder is selected from the group consisting of an autoimmune disorder, an autoimmune disorder characterized by excessive phagocytic activity, atherosclerosis, a cancer, an inflammatory disease or disorder, a lymphoproliferative disorder, an infectious disease, macrophage activation syndrome (MAS), a cytokine-related disorder, a central nervous system (CNS) disease, a disease of hyperactivated microglia, a disease of overactivated osteoclasts, osteoporosis, bone degradation and/or metastasis associated with cancer, multiple myeloma bone disease, systemic juvenile idiopathic arthritis, an allergy, a cancer with low PD-L1 expression, a cancer that is refractory to PD-1 and/or PD-L1 inhibitors, a disease or disorder characterized by multinucleated giant cells, and an atopic disease, optionally wherein:
(a) the bone degradation and/or metastasis associated with cancer results from a disease or disorder selected from multiple myeloma, breast cancer, and prostate cancer;
(b) the autoimmune disorder is lupus, rheumatoid arthritis, multiple sclerosis, hemophagocytic lymphohistiocytosis (HLH), or immune thrombocytopenia (ITP);
(c) the CNS disease is Alzheimer's disease, schizophrenia, or Huntington's disease;
(d) the disease or disorder is a lymphoproliferative disorder and the fusion molecule comprises a targeting moiety comprising rituximab or an antigen-binding fragment thereof;
(e) the disease or disorder is selected from the group consisting of atherosclerosis, a cancer, an inflammatory disease or disorder, and an infectious disease;
(f) the disease or disorder is selected from the group consisting of H. pylori infection, COVID, severe COVID, IgA nephropathy, and ovarian cancer;
(g) the disease or disorder characterized by multinucleated giant cells is selected from the group consisting of Langerhans cell histiocytosis and granulomas;
(h) the disease or disorder is selected from the group consisting of inflammatory bowel disease (IBD), Crohn's disease, ulcerative colitis, acute-on-chronic liver failure (ACLF), COVID, severe COVID, antineutrophil cytoplasmic antibody-associated vasculitis (AAV), autoimmune vasculitis, asthma, acute respiratory distress syndrome (ARDS), chronic obstructive pulmonary disease (COPD), Kawasaki disease, septic shock, a disease in which regulatory T cells express CD177, renal cell carcinoma, hepatocellular carcinoma, breast cancer, lung cancer, and colorectal cancer;
(i) the method further comprises administering a therapeutically effective amount of a chemotherapeutic agent to the subject, optionally wherein the chemotherapeutic agent is paclitaxel;
(j) the method further comprises administering a therapeutically effective amount of an immune checkpoint modulating agent to the subject, optionally wherein the immune checkpoint modulating agent is an anti-PD-1 agent or an anti-PD-L1 agent;
(k) the method further comprises administering therapeutically effective amounts of a chemotherapeutic agent and an immune checkpoint modulating agent to the subject, optionally wherein the chemotherapeutic agent is paclitaxel and the immune checkpoint modulating agent is an anti-PD-1 agent or an anti-PD-L1 agent; or
(l) the disease or disorder is ovarian cancer.

182. A method of identifying a modulator, enhancer, or inhibitor of the interaction of SMAGP and CLEC10A or CD177, the method comprising:
(a) measuring the binding of SMAGP to CLEC10A in the presence and absence of a test compound, wherein an increase or decrease in the amount of binding of SMAGP to CLEC10A in the presence of the test compound relative to the amount of binding of SMAGP to CLEC10A in the absence of the test compound identifies the test compound as a modulator of the interaction of SMAGP and CLEC10A, an increase in the amount of binding of SMAGP to CLEC10A in the presence of the test compound relative to the amount of binding of SMAGP to CLEC10A in the absence of the test compound identifies the test compound as an enhancer of the interaction of SMAGP and CLEC10A, and a decrease in the amount of binding of SMAGP to CLEC10A in the presence of the test compound relative to the amount of binding of SMAGP to CLEC10A in the absence of the test compound identifies the test compound as an inhibitor of the interaction of SMAGP and CLEC10A, optionally wherein the SMAGP and/or CLEC10A is expressed on the surface of cells or is a recombinant protein, further optionally wherein the recombinant protein is a fusion molecule comprising an Fc region; or
(b) measuring the binding of SMAGP to CD177 in the presence and absence of a test compound, wherein an increase or decrease in the amount of binding of SMAGP to CD177 in the presence of the test compound relative to the amount of binding of SMAGP to CD177 in the absence of the test compound identifies the test compound as a modulator of the interaction of SMAGP and CD177, an increase in the amount of binding of SMAGP to CD177 in the presence of the test compound relative to the amount of binding of SMAGP to CD177 in the absence of the test compound identifies the test compound as an enhancer of the interaction of SMAGP and CD177, and a decrease in the amount of binding of SMAGP to CD177 in the presence of the test compound relative to the amount of binding of SMAGP to CD177 in the absence of the test compound identifies the test compound as an inhibitor of the interaction of SMAGP and CD177, optionally wherein the SMAGP and/or CD177 is expressed on the surface of cells or is a recombinant protein, further optionally wherein the recombinant protein is a fusion molecule comprising an Fc region;
optionally wherein the test compound is selected from the group consisting of a small molecule, a polypeptide, or a nucleic acid, further optionally wherein the polypeptide is an antibody or the nucleic acid is a DNA or RNA aptamer,
optionally wherein the amount of binding is measured using enzyme linked immunosorbent assay (ELISA), surface plasmon resonance (SPR), or flow cytometry.

183. A method of treating a subject that has a tumor with an elevated level of CLEC10A expression as compared to a reference level of CLEC10A, the method comprising administering to the subject a therapeutically effective amount of a compound that antagonizes the binding of CLEC10A to SMAGP, optionally wherein the compound is the fusion molecule of claim 165, a polynucleotide comprising a nucleotide sequence encoding the fusion molecule, an expression vector comprising the polynucleotide, a nanoparticle comprising the fusion molecule, the polynucleotide, the expression vector, or a composition comprising any of the foregoing and a pharmaceutically acceptable carrier or excipient.

184. A method of:
(a) identifying a subject who will benefit from treatment with a fusion molecule that antagonizes the binding of CLEC10A to SMAGP, the method comprising determining the level of CLEC10A expression in a biological sample from the subject relative to a reference level of CLEC10A, wherein a higher level of CLEC10A expression in the biological sample compared to the reference level indicates that the subject will benefit from the treatment, optionally wherein the biological sample is a tumor sample, optionally wherein the level of CLEC10A expression in the biological sample is determined by immunohistochemistry, fluorescence in situ hybridization (FISH), or chromogenic in-situ hybridization (CISH), optionally wherein the CLEC10A is expressed on the surface of a myeloid cell and the SMAGP is expressed on the surface of a tumor cell; or
(b) identifying a subject who will benefit from treatment with a fusion molecule comprising a SMAGP extracellular domain (ECD), comprising measuring a level of CD177 expression in a biological sample from the subject and comparing to a reference level of CD177, wherein a higher level of CD177 expression compared to the reference level indicates the subject will benefit from the treatment, optionally wherein the biological sample is a tumor sample, optionally wherein the level of CD177 expression in the biological sample is determined by immunohistochemistry, fluorescence in situ hybridization (FISH), or chromogenic in-situ hybridization (CISH), optionally wherein the CD177 is expressed on the surface of a neutrophil or a regulatory T cell and the SMAGP is expressed on the surface of a tumor cell,
optionally further comprising administering to the subject a therapeutically effective amount of the fusion molecule of claim 165, a polynucleotide comprising a nucleotide sequence encoding the fusion molecule, an expression vector comprising the polynucleotide, a nanoparticle comprising the fusion molecule, the polynucleotide, the expression vector, or a composition comprising any of the foregoing and a pharmaceutically acceptable carrier or excipient.


SEQUENCE LISTING (63 sequences)

SEQ ID NO: 1 - protein, 36 aa, synthetic construct
MTSLLTTPSPREELMTTPILQPTEALSPEDGASTAL

SEQ ID NO: 2 - protein, 36 aa, synthetic construct
MNNLPATPSPEELMTTPVFQAPETLSPQAEEASTAL

SEQ ID NO: 3 - protein, 35 aa, synthetic construct
MTSLLTTPSPRELMTTPILRPTEALSPEDGASTAL

SEQ ID NO: 4 - protein, 5 aa, synthetic construct
  VARIANT 5: note = X is C or S
EPKSX

SEQ ID NO: 5 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 226 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 7 - protein, 227 aa, synthetic construct
DKTHTCPPCPAPELLGGPSVFLFPPKPKDTLMISRTPEVTCVVVAVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 226 aa, synthetic construct
DKTHTCPPCPAPELLGGPSVFLFPPKPKDTLMISRTPEVTCVVVAVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 9 - protein, 227 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 226 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 11 - protein, 227 aa, synthetic construct
DKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 226 aa, synthetic construct
DKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 13 - protein, 229 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 14 - protein, 228 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 15 - protein, 233 aa, synthetic construct
EPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 16 - protein, 232 aa, synthetic construct
EPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPG

SEQ ID NO: 17 - protein, 233 aa, synthetic construct
EPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 18 - protein, 232 aa, synthetic construct
EPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPG

SEQ ID NO: 19 - protein, 268 aa, synthetic construct
MTSLLTTPSPREELMTTPILQPTEALSPEDGASTALEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 267 aa, synthetic construct
MTSLLTTPSPREELMTTPILQPTEALSPEDGASTALEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 21 - protein, 268 aa, synthetic construct
MTSLLTTPSPREELMTTPILQPTEALSPEDGASTALEPKSSDKTHTCPPCPAPELLGGPSVFLFPPKPKDTLMISRTPEVTCVVVAVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 267 aa, synthetic construct
MTSLLTTPSPREELMTTPILQPTEALSPEDGASTALEPKSSDKTHTCPPCPAPELLGGPSVFLFPPKPKDTLMISRTPEVTCVVVAVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 23 - protein, 268 aa, synthetic construct
MTSLLTTPSPREELMTTPILQPTEALSPEDGASTALEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 267 aa, synthetic construct
MTSLLTTPSPREELMTTPILQPTEALSPEDGASTALEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 25 - protein, 268 aa, synthetic construct
MTSLLTTPSPREELMTTPILQPTEALSPEDGASTALEPKSSDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - protein, 267 aa, synthetic construct
MTSLLTTPSPREELMTTPILQPTEALSPEDGASTALEPKSSDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 27 - protein, 265 aa, synthetic construct
MTSLLTTPSPREELMTTPILQPTEALSPEDGASTALESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 28 - protein, 264 aa, synthetic construct
MTSLLTTPSPREELMTTPILQPTEALSPEDGASTALESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 29 - protein, 269 aa, synthetic construct
MNNLPATPSPEELMTTPVFQAPETLSPQAEEASTALEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 30 - protein, 268 aa, synthetic construct
MNNLPATPSPEELMTTPVFQAPETLSPQAEEASTALEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPG

SEQ ID NO: 31 - protein, 269 aa, synthetic construct
MNNLPATPSPEELMTTPVFQAPETLSPQAEEASTALEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 32 - protein, 268 aa, synthetic construct
MNNLPATPSPEELMTTPVFQAPETLSPQAEEASTALEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPG

SEQ ID NO: 33 - protein, 267 aa, synthetic construct
MTSLLTTPSPRELMTTPILRPTEALSPEDGASTALEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 266 aa, synthetic construct
MTSLLTTPSPRELMTTPILRPTEALSPEDGASTALEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 35 - protein, 4 aa, synthetic construct
  REPEAT 1..4: note = Repeats one or more times
GGGS

SEQ ID NO: 36 - protein, 4 aa, synthetic construct
  REPEAT 1..4: note = Repeats one or more times
SGGG

SEQ ID NO: 37 - protein, 10 aa, synthetic construct
  REPEAT 1..5: note = Repeats 0 to 4 times
  VARIANT 4..5: note = X is any amino acid that can be inserted into the sequence and not result in a polypeptide including the sequence GSG
GGGXXGGGGS

SEQ ID NO: 38 - protein, 10 aa, synthetic construct
  REPEAT 6..10: note = Repeats 0 to 4 times
  VARIANT 6: note = X is any amino acid that can be inserted into the sequence and not result in a polypeptide including the sequence GSG
GGGGSXGGGS

SEQ ID NO: 39 - protein, 10 aa, synthetic construct
  VARIANT 4: note = X is P
  VARIANT 5: note = X is S
  REPEAT 1..5: note = Repeats 0 to 4 times
GGGXXGGGGS

SEQ ID NO: 40 - protein, 10 aa, synthetic construct
  VARIANT 4: note = X is G
  VARIANT 5: note = X is Q
  REPEAT 1..5: note = Repeats 0 to 4 times
GGGXXGGGGS

SEQ ID NO: 41 - protein, 10 aa, synthetic construct
  VARIANT 4: note = X is G
  VARIANT 5: note = X is A
  REPEAT 1..5: note = Repeats 0 to 4 times
GGGXXGGGGS

SEQ ID NO: 42 - protein, 10 aa, synthetic construct
  VARIANT 6: note = X is P
  REPEAT 6..10: note = Repeats 0 to 4 times
GGGGSXGGGS

SEQ ID NO: 43 - protein, 15 aa, synthetic construct
GGGGAGGGGAGGGGS

SEQ ID NO: 44 - protein, 15 aa, synthetic construct
GGGGQGGGGQGGGGS

SEQ ID NO: 45 - protein, 15 aa, synthetic construct
GGGPSGGGPSGGGGS

SEQ ID NO: 46 - protein, 15 aa, synthetic construct
GGGGSPGGGSPGGGS

SEQ ID NO: 47 - protein, 5 aa, synthetic construct
GSGGS

SEQ ID NO: 48 - protein, 6 aa, synthetic construct
SGGSGS

SEQ ID NO: 49 - protein, 20 aa, synthetic construct
GGGGGSGGGGSGGGSGGGGS

SEQ ID NO: 50 - protein, 232 aa, synthetic construct
  VARIANT 5: note = X is C or S
EPKSXDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 51 - protein, 231 aa, synthetic construct
  VARIANT 5: note = X is C or S
EPKSXDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 52 - protein, 222 aa, synthetic construct
CPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 53 - protein, 221 aa, synthetic construct
CPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 54 - protein, 217 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 55 - protein, 216 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 56 - protein, 209 aa, synthetic construct
SVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 57 - protein, 208 aa, synthetic construct
SVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 58 - protein, 10 aa, synthetic construct
  VARIANT 5: note = X is C or S
EPKSXDKTHT

SEQ ID NO: 59 - protein, 15 aa, synthetic construct
  VARIANT 5: note = X is C or S
EPKSXDKTHTCPPCP

SEQ ID NO: 60 - protein, 20 aa, synthetic construct
  VARIANT 5: note = X is C or S
EPKSXDKTHTCPPCPAPELL

SEQ ID NO: 61 - protein, 23 aa, synthetic construct
  VARIANT 5: note = X is C or S
EPKSXDKTHTCPPCPAPELLGGP

SEQ ID NO: 62 - protein, 37 aa, synthetic construct
MTSLLTTPSPREELMTTPILQPTEALSPEDGASTALK

SEQ ID NO: 63 - protein, 37 aa, synthetic construct
TRTTLIMESTTPALMLLEPLGSPQDAPTSTEEAPSLK